(19) United States
(12) Patent Application Publication
(10) Pub. No.: US 2026/0159573 A1
(43) Pub. Date: Jun. 11, 2026

(54) AFUCOSYLATION MODIFICATION

(71) Applicant: Genentech, Inc., South San Francisco, CA (US)

(72) Inventors: Natarajan Vijayasankaran, Shrewsbury, MA (US); Gavin Christian Barnard, Bloomington, IN (US); Jason Goodrick, San Francisco, CA (US); Shahram Misaghi, Oakland, CA (US); Eleanor Oates, San Carlos, CA (US)

(73) Assignee: Genentech, Inc., South San Francisco, CA (US)

(21) Appl. No.: 19/394,272

(22) Filed: Nov. 19, 2025

Related U.S. Application Data
(60) Provisional application No. 63/725,767, filed on Nov. 27, 2024.

(51) Int. Cl. C07K 16/00

(52) U.S. Cl. CPC C07K 16/00; C07K 2317/14; C07K 2317/41

(57) ABSTRACT

The present application relates to methods of producing a protein in fucosylated and afucosylated forms at a predetermined ratio.

### CROSS-REFERENCE TO RELATED APPLICATIONS

The present application claims benefit of U.S. Provisional Application No. 63/725,767, filed Nov. 27, 2024, the disclosure of which is incorporated by reference herein in its entirety.

### SEQUENCE LISTING

This application contains a Sequence Listing, which has been submitted electronically in XML format and is hereby incorporated by reference in its entirety. Said XML copy, created on Nov. 19, 2025, is named P39135-US-1_Sequence_Listing.xml and is 9,857 bytes in size.

### TECHNICAL FIELD

The present application relates to methods of producing fucosylated and afucosylated forms of a protein at a predetermined ratio.

### BACKGROUND

Antibody-type molecules, including monoclonal antibodies (mAbs) are the most commonly used biotherapeutics for treating a variety of different diseases ranging from cancer to autoimmunity, and genetic disorders. Following the expression of proteins in eukaryotic, e.g. mammalian host cells, the proteins undergo post-translational modifications, often including the enzymatic addition of sugar residues, generally referred to as “glycosylation”. One type of glycosylation is fucosylation.

Fucosylation of glycans requires synthesis of GDP-fucose via the de novo or salvage pathway. The fucosylation process involves sequential function of several enzymes resulting in addition of a fucose molecule to the first N-acetylglucosamine (GlcNAc) moiety of the reducing end of a glycan (Becker & Lowe, Glycobiology, 13, 2003, 41R-53R). Laboratories of Lowe and Fukuda discovered the two key enzymes of the de novo pathway responsible for production of GDP-fucose from mannose and/or glucose. Specifically, the laboratories discovered, GDP-D-mannose-4,6-dehydratase (GMD) and GDP-keto-6-deoxymannose-3,5-epimerase,4-reductase (FX) (Ohyama et al., J Biol Chem, 273, 1998, 14582-14587; Smith et al., J Cell Biol, 158, 2002, 801-815; and Becker, Genetic and Biochemical Determination of Fucosylated Glycan Expression, 2002, Thesis from The University of Michigan, UMI3121891). In the absence of fucose, GMD and FX convert mannose and/or glucose to GDP-fucose. In turn, GDP-fucose, is transported into the Golgi complex where nine fucosyl-transferases (FUT1-9) act in concert to fucosylate the first GlcNAc molecule of a glycan (Becker & Lowe, Glycobiology, 13, 2003, 41R-53R). In the presence of fucose, however, fucose-kinase and GDP-fucose pyrophosphorylase can convert fucose into GDP-fucose, bypassing the need for de novo synthesis of GDP-fucose by GMD and FX enzymes (Becker & Lowe, Glycobiology, 13, 2003, 41R-53R).

Expression of antibody-dependent cellular cytotoxicity (ADCC) activity and complement-dependent cytotoxicity activity (CDC) activity of human IgG1 subclass antibodies requires binding of the Fc region of antibody to an antibody receptor present on the surface of an effector cell, such as a killer cell, a natural killer cell, an activated macrophage or the like (hereinafter referred to as “FcγR”) and various complement components. Reducing or inhibiting N-glycan fucosylation of antibodies, or Fc-fusion proteins, can enhance the ADCC activity. ADCC typically involves the activation of natural killer (NK) cells and is dependent on the recognition of antibody-coated cells by Fc receptors on the surface of the NK cell. Binding of the Fc domain to Fc receptors on the NK cells is affected by the glycosylation state of the Fc domain. In addition, the type of the N-glycan at the Fc domain also affects ADCC activity. Therefore, for an antibody composition, or a Fc-fusion protein composition, an increase of the relative amount of afucosyl N-glycans can enhance the binding affinity for an FcγRIII, or ADCC activity of the composition.

For therapeutic use, antibodies are typically expressed using Chinese hamster ovary (CHO) cells due to the fact that antibody glycosylation, which plays an important role in their effector function, is maintained in this expression system. Specifically, the fucosylation level of glycan moieties in antibodies (e.g. monoclonal antibodies) can be determinant of their therapeutic mode of action (MOA). Lack of fucose (afucosylated) in the glycan moieties can increase recruitment of Natural Killer (NK) cells to the target cells by 10 to 100 fold. Therefore, controlling levels of afucosylated antibody (e.g., monoclonal antibody) species during the manufacturing process is critical for maintaining or tuning its NK cells mediated MOA. Various genetic engineering approaches, such as knocking out (KO) the terminal fucose-transferring FUT8 gene, or addition of small molecule inhibitors (ex. 2F-Peracetyl-Fucose or 2FP) have been employed to generate afucosylated monoclonal antibodies. However, use of many of these approaches can pose major challenges to the manufacturing process. For example, using small molecule inhibitors such as 2FP can result in non-specific conjugation of these small molecules to certain amino acids, which may result in reduced efficacy, stability, or even worse, immunogenicity. FUT8 knockout (KO) CHO cell lines, on the other hand, can only express fully afucosylated mAbs and hence cannot be used to generate a desired percentage of afucosylated mAbs. Therefore, to achieve desired levels of afucosylated mAbs both wild type (WT) and FUT8 KO cell lines have to be developed, and the products of manufacture mixed in the desired amount. This poses major manufacturing challenges in the form of identifying cell lines with comparable product quality attributes (PQAs) and accommodating double the number of manufacturing runs to fulfil drug supply demands.

WO2017079165 (Genentech Inc.) discloses methods for producing a protein with a desired ratio of fucosylated and afucosylated forms, by expressing the protein in a host cell which comprises substantially no GDP-keto-6-deoxymannose-3,5-epimerase,4-reductase (FX) activity due to knock-out of the FX gene.

In an FX KO host, the de-novo fucose generation pathway is compromised by knocking out the FX gene while the salvage fucose pathway still remains intact. Therefore, a FX KO host is capable of expressing either fully afucosylated, or fully fucosylated monoclonal antibodies simply by adding fucose to the production media. Since the same host can be used to express either fucosylated or afucosylated monoclonal antibodies with comparable PQAs, the FX KO technology overcomes the need for generating and screening hundreds of WT or FUT8 KO cell lines, which is required for generation of fucosylated and afucosylated mAb(s) with comparable PQAs. Nevertheless, even FX KO technology requires two separate manufacturing runs in order to generate both fucosylated and afucosylated mAbs. The fucosylated and afucosylated drug products then need to be mixed post purification in order to achieve a desired level of afucosylated mAb. Adding to the complexity of the process, depending on the levels of afucosylated mAb, different numbers or scales of manufacturing runs may be needed in order to achieve the desired outcome.

There remains a need to be able to more efficiently produce a protein of interest, e.g. a monoclonal antibody, with a pre-determined/desired ratio of fucosylated to afucosylated forms.

### SUMMARY OF THE INVENTION

Described herein are process development methods that when used in combination with FX KO technology, can robustly and reproducibly generate the desired levels of a desired afucosylated/fucosylated protein (e.g., monoclonal antibody) from a single production run. In part this can be achieved by strategic addition of fucose, at or above saturation levels, on various days during the production process. It was also found that the desired levels of afucosylated monoclonal antibody may be approximated depending on the specific productivity (Qp) of the FX KO clone, and fucose addition day can then be experimentally confirmed using small scale production runs.

The inventors have also discovered that gene engineering can be employed to generate partial knockouts of the FX gene, and such partial FX KO cells produce a ratio of afucosylated/fucosylated forms of an expressed protein depending on the degree of FX KO. Additionally, the inventors have found that FACS analysis based on Lens culinaris agglutinin (LCA) conjugated to FITC (LCA-FITC) can be used early during clone screening process to identify clones with varying levels of partial KO of the FX gene. As shown herein, partial FX KO clones can reproducibly express a fixed level of afucosylated monoclonal antibody, ranging from about 10-90%, preferably ranging from about 20-80%, more preferably ranging about 25-75%, during production, without the need to add fucose to the culture medium.

Accordingly, partial FX knockout can be utilized to produce isolated clones capable of expressing mAb(s) (or other proteins) with a desired level of afucosylation without a need to add fucose or changing any of the production parameters. Should the level of afucosylation require adjusting from the level produced by a selected partial FX KO manufacturing cell clone, this can be effected by the selective alteration of the amount of fucose in the culture medium.

According to a first aspect of the invention here is provided a method of producing a protein with a desired ratio of fucosylated to afucosylated forms, the method comprising:
(a) culturing a host cell capable of expressing a protein, or expressing a protein, in a culture medium under conditions suitable to express the protein, wherein the host cell has been engineered to (i) produce the protein, and (ii) to possess detectable but reduced GDP-keto-6-deoxymannose-3,5-epimerase,4-reductase (FX) activity compared to wild type, wherein said host cell expresses said protein, and wherein the amount of FX activity in the host cell determines the ratio of fucosylated to afucosylated forms of the expressed protein.

Suitably, the method further comprises:
b) determining the level of fucosylation of the expressed protein; and
c) adjusting the amount of the fucose source in the culture medium so that the protein is produced with the desired level of fucosylation (desired ratio of fucosylated to afucosylated forms).

In certain embodiments, the host cell is not a temperature-sensitive FX mutant host cell. In certain other embodiments, the host cell is not a null FX mutant. In certain other embodiments, the host cell does not comprise an FX knockout in both alleles.

According to a second aspect, provided herein is a method of producing a protein, wherein the protein is produced in fucosylated and afucosylated forms in a desired ratio, comprising: culturing a host cell capable of expressing the protein in fucosylated and afucosylated forms in a desired or near-desired ratio in a culture medium, wherein the host cell has a partial knock-out of the FX gene, optionally wherein the near-desired ratio of fucosylated to afucosylated forms is adjusted to the desired ratio by increasing or decreasing the amount of fucose in the culture medium.

According to a third aspect, provided herein is a method of producing a cell clone capable of expressing a protein, wherein the protein can be produced in fucosylated and afucosylated forms in a desired ratio, comprising:
A.
   (i) subjecting isolated host cells capable of expressing the protein to gene editing to produce individual gene-edited host cell clones with partial knock-out of the FX gene;
   (ii) selecting one or more gene-edited host cell clones which produce the protein in fucosylated and afucosylated forms at the desired ratio; or
B.
   (i) transfecting a clonal population of isolated host cells that have been gene edited to have reduced FX activity, compared to wild type (unmodified) host cells, with a polynucleotide sequence encoding said protein, wherein the isolated host cells have been selected for a particular level of FX activity that results in said protein being produced in a particular ratio of fucosylated and afucosylated forms, to produce transfected host cells;
   (ii) selecting one or more transfected that express the protein of interest in fucosylated and afucosylated forms at a desired ratio.

According to a fourth aspect, provided herein are cell clones produced by the method of the third aspect.

According to a fifth aspect, provided herein is a cell culture comprising one or more cell clone(s) capable of expressing the protein, or expressing the protein, produced by the method of the third aspect of the invention, and a culture medium, the culture medium optionally comprising a source of fucose.

According to a sixth aspect, provided herein is a method of producing a protein, wherein the protein is produced in fucosylated and afucosylated forms at a desired ratio, comprising
(i) determining the period of time required for culturing a host cell having substantially no FX activity in a culture medium lacking a fucose source prior to culturing said host cell in a culture medium comprising a fucose source sufficient to produce fucosylated and afucosylated forms of the protein in a desired ratio;
(ii) culturing the host cell having substantially no FX activity in a culture medium lacking a fucose source for the period of time determined in step (i); and
(iii) culturing the host cell in a culture medium comprising a fucose source for a period of time sufficient to produce the protein in fucosylated and afucosylated forms at the desired ratio.

Suitably, the protein (or protein of interest) in any of the aspects of the invention is an Fc-containing protein, such as an antibody molecule, e.g. a monoclonal antibody (mAb).

In certain embodiments of any of the aspects herein, the host cell is not a temperature-sensitive FX mutant host cell. In certain other embodiments of any of the aspects herein, the host cell is not a null FX mutant. In certain other embodiments of any of the aspects herein, the host cell does not comprise an FX knockout in both alleles.

These and other aspects and advantages of the present invention will become apparent from the subsequent detailed description and the appended claims. It is to be understood that one, some, or all of the properties of the various embodiments described herein may be combined to form other embodiments of any aspect of the present invention.

### BRIEF DESCRIPTION OF THE FIGURES

FIG. 1A and FIG. 1B. Overall clone generation: Partial knockout (KO) of the FX (FXpKO) gene can be identified and monitored by FACS analysis. FIG. 1A) Schematic overview of various FX KO strategies. Partial KO of the FX gene results in generation of clones with different levels of FX gene expression, which results in expression of different levels of mAb-1 afucosylation depending on the KO degree and the level of FX gene expression. For the full FX KO clones, addition of fucose starting at different days from the start of production culture controls afucosylated antibody levels. Alternatively, separate production runs can be used to generate fully fucosylated or afucosylated antibody batches, which can be mixed at the certain ratios to obtain the desired levels of afucosylated mAb-1. FIG. 1B) Examples representative of using LCA-FITC staining and FACS profile analysis to identify clones with partial KO of the FX (FXpKO) gene with high or low levels of afucosylated antibodies. WT and FX KO hosts were used as controls for LCA-FITC staining profiles, respectively. Percentage of afucosylated mAb-1 levels was determined by analysis of AMBR®15 production cultures.

FIG. 2A, FIG. 2B, and FIG. 2C. Wide range FACS screening (mAb-1): Partial KO of the FX gene can result in the isolation of clones with different levels of mAb-1 afucosylation. FIG. 2A) Wide range FACS screening (mAb-1). LCA-FITC FACS analysis of clones with partial KO of the FX gene obtained from Cas9 mediated targeting of FX gene. Levels of % afucosylated mAb-1 were determined based on AMBR®15 production. WT and FX KO hosts were used as controls for LCA-FITC staining profiles, respectively. FIG. 2B) End of production afucosylation vs. titer. Graph of mAb-1 titer and % afucosylation levels measured in AMBR®15 production assay for clones with partial KO of the FX gene. FIG. 2C) Clone screening product quality (mAb-1). Table of titer (g/L), viable cell count (VCC) (10⁶ cells/ml), % viability, and major glycan species for each partial FX KO clone tested.

FIG. 3A and FIG. 3B. Full FXKO screening (mAb-1): Evaluation of mAb-1 expressing clones with complete KO of the FX gene. FIG. A) Six clones with complete KO of FX genes were cultured in the absence (left FACS plot) or presence (right FACS plot) of 1 mM fucose in the media for 2 days and cultures were analyzed by FACS using LCA-FITC surface staining. WT and FXKO hosts were used as controls for LCA-FITC staining profile. FIG. B) PCR analysis for detection of WT and FXKO alleles within the selected 6 mAb-1 expressing clones with complete KO of the FX gene. WT and FXKO hosts were used as controls.

FIG. 4A, FIG. 4B, FIG. 4C, FIG. 4D, and FIG. 4E. Full FXKO clone 1 (mAb-1): Addition of fucose at different days during AMBR®15 production for mAb-1 expressing FXKO clone 1. FIG. 4A) Titer (g/L), FIG. B) IVCC (10⁸ cells/ml), and FIG. 4C) % afucosylated glycan species for culture where 1 mM fucose was added starting at different days (and subsequent feeds) during production. FIG. 4D) Table of titer (g/L), VCC (10⁶ cells/ml), % viability, and major glycan species for each fucose addition day for the mAb-1 expressing FXKO clone 1. FIG. E) Comparison of charge and size variant species for some of the FX KO samples to the WT reference materials.

FIG. 5A, FIG. 5B, FIG. 5C, FIG. 5D, and FIG. 5E. Full FXKO clone 4 (mAb-1): Addition of fucose at different days during AMBR®15 production for mAb-1 expressing FXKO clone 4. FIG. 5A) Titer (g/L), FIG. 5B) IVCC (10⁸ cells/ml), and FIG. 5C) % Afucosylated glycan species for culture where 1 mM fucose was added starting at different days (and subsequent feeds) during production. FIG. 5D) Table of titer (g/L), VCC (10⁶ cells/ml), % viability, and major glycan species for each fucose addition day for the mAb-1 expressing FXKO clone 4. FIG. 5E) Comparison of charge and size variant species for some of the FX KO samples to the WT reference materials.

FIG. 6A, FIG. 6B, FIG. 6C, and FIG. 6D. Full FXKO clone 5 (mAb-1): Addition of fucose at different days during AMBR®15 production for mAb-1 expressing FXKO clone 5. FIG. 6A) Titer (g/L), FIG. 6B) IVCC (10⁸ cells/ml), and FIG. 6C) % afucosylated glycan species for culture where 1 mM fucose was added starting at different days (and subsequent feeds) during production. FIG. 6D) Table of titer (g/L), VCC (10⁶ cells/ml), % viability, and major glycan species for each fucose addition day for the mAb-1 expressing FX KO clone 5. Note: due to contamination Day 1 fucose addition for clone 5 was not analyzed.

FIG. 7A, FIG. 7B, FIG. 7C, and FIG. 7D. Low partial FX KO clone 44 (mAb-1): Addition of fucose at different days during AMBR®15 production for mAb-1 expressing Low partial-FXKO clone 44. FIG. 7A) Titer (g/L), FIG. 7B) IVCC (10⁸ cells/ml), and FIG. 7C) % Afucosylated glycan species for culture where 1 mM fucose was added starting at different days (and subsequent feeds) during production. FIG. 7D) Table of titer (g/L), VCC (10⁶ cells/ml), % viability, and major glycan species for each fucose addition day for the mAb-1 expressing Low partial-FX KO clone 44.

FIG. 8A, FIG. 8B, FIG. 8C, and FIG. 8D. Mid partial FXKO clone 7 (mAb-1): Addition of fucose at different days during AMBR®15 production for mAb-1 expressing Mid partial-FXKO clone 7. FIG. 8A) Titer (g/L), FIG. 8B) IVCC (10⁸ cells/ml), and FIG. 8C) % afucosylated glycan species for culture where 1 mM fucose was added starting at different days (and subsequent feeds) during production. FIG. 8D) Table of titer (g/L), VCC (10⁶ cells/ml), % viability, and major glycan species for each fucose addition day for the mAb-1 expressing Mid partial-FX KO clone 7.

FIG. 9A and FIG. 9B. Comparing afucosylation rates (FIG. 9A) and specific productivities (FIG. 9B) of clones with Full FXKO (clone 1) and partial FX KO with Mid-(clone 7) and Low-(clone 44) levels of FX gene. FIG. 9A) Percent levels of afucosylated mAb-1 antibodies are depicted. FIG. 9B) Total specific productivity of clones over the duration of the production process.

FIG. 10A, FIG. 10B, FIG. 10C, FIG. 10D, and FIG. 10E. Full FX KO clone 9616 (mAb-2): Addition of fucose at different days during AMBR®15 production for mAb-2 expressing full FXKO clone 9616. FIG. 10A) Titer (g/L), FIG. 10B) IVCC (10⁸ cells/ml), and FIG. 10C) % afucosylated glycan species for culture where 1 mM fucose was added starting at different days (and subsequent feeds) during production. FIG. 10D) Table of titer (g/L), VCC (10⁶ cells/ml), % viability, and major glycan species for each fucose addition day for the mAb-2 expressing full FX KO clone 9616. FIG. 10E) Comparison of charge and size variant species of the mAb-2 FXKO samples with no fucose feed as well as different daily fucose feeds.

FIG. 11. Full FX KO Clone 4 (mAb-2)—Mixing Experiment. Harvested cell culture fluid (HCCF) mixing experiment of fully afucosylated and fucosylated culture samples. Clone 4 production HCCF samples with no fucose feed (fully afucosylated mAb-1) and DO fucose feed (fully fucosylated mAb-1) were mixed at 1:3, 1:1, and 3:1 ratio and % total afucosylated species were measured following PhyTip protein A column purification.

### DETAILED DESCRIPTION OF THE INVENTION

The present application utilizes the principle that a host cell possessing partial knockout of GDP-keto-6-deoxymannose-3,5-epimerase,4-reductase (FXpKO) is capable of producing a ratio of fucosylated and afucosylated forms of an antibody when cultured in a culture medium. The ratio is dictated by the degree of partial knock out. It was found that expression of an antibody in various FxpKO cells in the production media allows for the production of an antibody with a particular ratio of fucosylated and afucosylated glycan forms depending on the percentage of partial KO, without any significant change to the antibody titer or product quality. Clones of the FXpKO host cell expressed the antibody with relatively high specific productivities and good growth profiles.

The present application also utilizes the principle that a GDP-keto-6-deoxymannose-3,5-epimerase,4-reductase full knockout (FXKO) cell is capable of producing both fucosylated and afucosylated forms of an antibody when cultured in a culture medium comprising varying amounts of fucose. The inventors have found that by selective addition of fucose to the production media after an initial period of growth without fucose allows for the production of an antibody with a desired ratio of fucosylated and afucosylated glycan forms without any change to the antibody titer or product quality. By altering the period of growth without and then with the fucose for a particular manufacturing process the ratio of fucosylated and afucosylated glycan forms can be altered and thus the process conditions (i.e., when fucose is present in the medium) can be tailored or pre-designed to ensure production of a desired ratio of fucosylated and afucosylated glycan forms.

The present application thus provides methods for producing fucosylated and afucosylated proteins from the same FXKO or FXpKO host cell. This is advantageous as it allows for the production of fucosylated and afucosylated forms of a protein with similar product qualities.

The FXpKO and FXKO clones may be utilized to express any antibody or Fc-containing polypeptide with a therapeutically relevant ratio of fucosylated and afucosylated glycoforms. It has been reported that while afucosylated antibodies enhance ADCC-mediated cell death via mononuclear cells (such as NK cells), polymorphonuclear (PMN) cells preferentially recognize and kill their targets via binding to antibodies with fucosylated glycans (Peipp et al., Blood, 112, 2008, 2390-2399). Thus, the ability to produce a desired ratio of fucosylated and afucosylated forms of an antibody should allow for production of a therapeutic protein (e.g. antibody or Fc-containing polypeptide) with the desired level of ADCC function while minimizing any possible ADCC-linked toxicity.

### Definitions

A fucosylated form of a protein, as used herein, refers to a glycan structure having at least one fucose moiety.

An afucosylated form of a protein, as used herein, refers to a glycan structure lacking any fucose moiety.

“Fc-containing protein,” as used herein, refers to a protein (e.g., an antibody or a Fc-containing fusion protein) comprising a Fc domain. In some embodiments, the Fc-containing protein comprises one or more protein subunits. In some embodiments, the Fc-containing protein comprises one or more polypeptides.

“Fc domain,” as used herein, refers to the “fragment crystallizable” (Fc) region of an immunoglobulin heavy chain or a C-terminal fragment thereof. The term includes wild type Fc domains and variant Fc domains. In some embodiments, the human IgG heavy chain Fc domain extends from Cys226, or from Pro230, to the carboxyl-terminus of the heavy chain (amino acid number is according to the EU numbering system, also called the EU index, as described in Kabat et al., Sequences of Proteins of Immunological Interest, 5th Ed. Public Health Service, National Institutes of Health, Bethesda, MD, 1991). In some embodiments, the term includes a C-terminal fragment of an immunoglobulin heavy chain and one or more constant regions. In some embodiments, for IgG, the Fc domain may comprise immunoglobulin domains CH2 and CH3 and the hinge between CHI and CH2.

As used herein, “Fc-containing fusion protein” refers to a protein comprising a Fc domain fused to at least one other heterologous protein unit or polypeptide.

The term “heavy chain” used herein refers to an immunoglobulin heavy chain.

Antibodies are glycoproteins, with glycosylation in the Fc region. Thus, for example, the Fc region of an IgG immunoglobulin is a homodimer comprising interchain disulfide-bonded hinge regions, glycosylated CH2 domains bearing N-linked oligosaccharides at asparagine 297 (Asn-297), and non-covalently paired CH3 domains. Glycosylation plays an important role in effector mechanisms mediated by Fc receptors, including FcγRI, FcγRII, FcγRIII, and Clq. Thus, antibody fragments of the present invention must include a glycosylated Fc region and an antigen-binding region.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they comprise a Fc domain.

The terms “full length antibody,” is used herein to refer to an antibody having a structure substantially similar to a native antibody structure or having heavy chains that contain an Fc region as defined herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “chimeric” antibody refers to an antibody in which a portion of the heavy and/or light chain is derived from a particular source or species, while the remainder of the heavy and/or light chain is derived from a different source or species.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. As used herein, “human antibody” includes such antibodies wherein the antibodies are produced recombinantly, but does not include antibodies actually produced by (e.g., isolated from) a human.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., complementarity determining regions (CDRs)) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous” compared to a constant region of an antibody), and an immunoglobulin constant domain sequence (e.g., CH2 and/or CH3 sequence of an IgG). Exemplary adhesin sequences include contiguous amino acid sequences that comprise a portion of a receptor or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, including phage display techniques and high throughput sorting methods. The immunoglobulin constant domain sequence in the immunoadhesin can be obtained from any immunoglobulin, such as IgG1, IgG2, IgG3, or IgG4 subtypes, IgA (including IgA1 and IgA2), IgE, IgD, or IgM.

The term “therapeutic antibody” refers to an antibody that is used in the treatment of disease. A therapeutic antibody may have various mechanisms of action. A therapeutic antibody may bind and neutralize the normal function of a target associated with an antigen. For example, a monoclonal antibody that blocks the activity of the protein needed for the survival of a cancer cell causes the cell's death. Another therapeutic monoclonal antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen. Exemplary therapeutic antibodies produced using the methods of the disclosure are further described below.

The terms “Fc receptor” and “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITAM) in its cytoplasmic domain (see review M. in Daeron, Annu. Rev. Immunol. 15:203-234 (1997)). FcRs are reviewed in Ravetch and Kinet, Annu. Rev. Immunol 9:457-92 (1991); Capel et al, Immunomethods 4:25-34 (1994); and de Haas et al, J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et ah, J. Immunol. 117:587 (1976) and Kim et ah, J. Immunol. 24:249 (1994)) and mediates slower catabolism, thus longer half-life.

“Effector function” refers to those biological activities attributable to the Fc region of an antibody, which vary with the antibody isotype. Examples of antibody effector functions include: Clq binding and complement-dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

“Antibody-dependent cell-mediated cytotoxicity” and “ADCC” refer to a cell-mediated reaction in which nonspecific cytotoxic cells that express Fc receptors (FcRs) {e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al, PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et ah, J. Immunol. Methods 202: 163 (1996), may be performed.

“Host cell,” as used herein, refers to a cell capable of producing a protein or polypeptide product. In some embodiments, the host cell can produce a Fc-containing protein.

As used herein, “substantially no FX activity” refers to a reduction of an activity level of FX or inactivation of the FX gene as compared to a host cell comprising wild type FX without a reduction of the activity level, wherein the reduction is by at least about 80%, 85%, 90%, 95%, or 100%. In some embodiments, the activity level is reduced by about 80% to 100%, about 85% to 100%, about 90% to 100%, or about 95% to 100%. In some embodiments, the activity level is reduced by at least 95%. In some embodiments, the activity level is reduced by 100%. In some embodiments, the activity level of the enzyme is no more than 20%, as compared to a host cell comprising a wild type enzyme without a reduction of the activity level of the enzyme. In some embodiments, the activity level of the enzyme is no more than 15%, as compared to a host cell comprising a wild type enzyme without a reduction of the activity level of the enzyme. In some embodiments, the activity level of the enzyme is no more than 10%, as compared to a host cell comprising a wild type enzyme without a reduction of the activity level of the enzyme. In some embodiments, the activity level of the enzyme is no more than 5%, as compared to a host cell comprising a wild type enzyme without a reduction of the activity level of the enzyme.

The phrases “disruption of the gene” and “gene disruption” refer to a mutation of the native, endogenous DNA sequence and/or the promoter region of a gene so as to decrease or prevent expression of that gene in the cell as compared to the wild type or naturally occurring sequence of the gene.

The term “knockout” or alternately “inactivation” refers to an alteration in the nucleic acid sequence of a gene that reduces the biological activity of the polypeptide normally encoded therefrom. Full or substantially full KO (FXKO) reduces the biological activity of the polypeptide normally encoded therefrom by at least 80%, such as at least 85%, at least 90%, at least 95%, at least 98%, at least 99% or 100% compared to the unaltered gene. A partial KO (FXpKO) reduces the biological activity of the polypeptide normally encoded therefrom by less than 80%, such as by about 5%, 10%, 15%, 20%, 25%, 30%, 35%, 40%, 45%, 50%, 55%, 60%, 65%, 70%, or 75% compared to the unaltered gene. Suitably, a partial KO reduces the biological activity of the polypeptide normally encoded therefrom by about 20% to about 80%, such as between about 25-75%. The alteration resulting in such a partial knockout, for example, may be an insertion, substitution, deletion, frameshift mutation, or missense mutation.

The term “knockdown” refers to techniques by which the expression of one or more genes is reduced, either through genetic modification (a change in the DNA of one of the organism's chromosomes), such as using gene editing techniques (such as with CRISPR-CAS, TALENs or Zinc finger techniques), or by treatment with a reagent such as a short DNA or RNA oligonucleotide with a sequence complementary to either an mRNA transcript or a gene. If genetic modification of DNA is done, the result is a “knockdown organism” or “knockdown host cell”.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extra chromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” protein is one which has been separated from a component of its natural environment. In some embodiments, a protein is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For example, for review of methods for assessment of antibody purity, see, e.g., Flatman et al., J. Chromatogr. B 848:79-87 (2007).

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic, or substantially nontoxic, to a subject, A pharmaceutically acceptable carrier includes, but is not limited to, a buffer, excipient, stabilizer, or preservative.

As used herein, “treatment”” or ““treating”” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The methods of the invention contemplate any one or more of these aspects of treatment.

The term ““individual”” or “subject” refers to a mammal and includes, but is not limited to, human, bovine, horse, feline, canine, rodent, or primate. In some embodiments, the individual is a human.

A “therapeutically effective amount” is at least the minimum concentration required to effect a measurable improvement of a particular disorder or disease, or to effect treatment of the disorder or disease. A therapeutically effective amount herein may vary according to factors such as the disease state, age, sex, and weight of the patient, and the ability of the antibody to elicit a desired response in the individual. A therapeutically effective amount is also one in which any toxic or detrimental effects of a drug, e.g., an antibody, are outweighed by the therapeutically beneficial effects. A “prophylactically effective amount” refers to an amount effective, at the dosages and for periods of time necessary, to achieve the desired prophylactic result. Typically, but not necessarily, since a prophylactic dose is used in subjects prior to or at early stage of disease, the prophylactically effective amount can be less than the therapeutically effective amount.

As used herein, “intensified fed-batch process” cell culture is a biomanufacturing strategy that increases efficiency by using a high-density seed culture to shorten the main production time, and essentially uses high density inoculation followed by fed-batch cultivation. An intensified fed-batch process may use an initial seeding cell density of >2×10⁶ cells per milliliter. This can be achieved by implementing N-1 perfusion seed culture, which can achieve a much higher final viable cell density than conventional batch N-1 seed culture.

It is understood that aspects and embodiments of the invention described herein include “consisting”” and/or “consisting essentially of” aspects and embodiments.

Reference to “about” a value or parameter herein includes (and describes) variations that are directed to that value or parameter per se. For example, a description referring to “about X” includes a description of “X.” The term “about”, as used herein, in connection with a numerical value, means said value±1%, ±2%, ±3%, ±4%, 5%, ±6%, ±7%, 8%, ±9%, or ±10%.

As used herein and in the appended claims, the singular forms “a,” “or,” and “the” include plural referents unless the context clearly dictates otherwise.

### Methods of Producing Fucosylated and Afucosylated Forms of a Protein

Molecular biological and protein science techniques for cloning of nucleic acids, transfecting/transforming said nucleic acids into suitable host cells so as to facilitate expression of a heterologous protein of interest, selection of appropriate culture media and culturing conditions needed to express, and optionally isolate/purify the expressed protein of interest, are well known to the person of skill in the art. Such techniques are well described in standard text books, such as in Sambrook et al., Molecular Cloning: A Laboratory Manual, 2nd ed., Cold Spring Harbor Laboratory, Cold Spring Harbor, NY (1989).

According to the first aspect of the invention there is provided a method of producing a protein of interest with a desired ratio of fucosylated to afucosylated forms, the method comprising:
(a) culturing a host cell capable of expressing the protein of interest in a culture medium under conditions suitable to express the protein of interest, wherein the host cell has been engineered to (i) produce the protein of interest, and (ii) to possess detectable but reduced GDP-keto-6-deoxymannose-3,5-epimerase,4-reductase (FX) activity compared to wild type, wherein the amount of FX activity in the host cell determines the ratio of fucosylated to afucosylated forms of the expressed protein produced.

In all embodiments herein, the cell has some FX activity. In particular, host cells with zero (nil) FX activity (full FX knock out) are excluded.

Suitably, the host cell has been engineered to possess about, or less than, 5%, 10%, 15%, 20%, 25%, 30%, 35%, 40%, 45%, 50%, 55%, 60%, 65%, 70%, 75% FX activity, or about, or less than, 80% FX activity, compared to wild-type activity, but not zero activity. AS used herein, “wild-type activity” means the amount of FX activity that the cell possesses prior to the engineering (e.g., FX gene-editing) of the cell to possess the reduced FX activity.

In a particular embodiment FX activity in the engineered host cell is reduced to between 20% and 80% of wild-type activity, i.e. the activity of the engineered cell is reduced so that the activity is between about 10% and 90% of the wild-type activity, or, the activity is between about 20% to 80% of the wild-type activity; or between 10% and 20%, between 15% and 25%, between 20% and 30%, between 25% and 35%, between 30% and 40%, between 35% and 45%, between 40% and 50%, between 45% and 55%, between 50% and 60%, between 55% and 65%, between 60% and 70%, between 65% and 75%, between 70% and 80%, between 75% and 85%, or between 80% and 90% of the wild-type activity of the host cell.

In a particular embodiment the reduced FX activity is to between 25% and 75% of wild-type activity.

In a particular embodiment the reduced FX activity is to between 30% and 75% of wild-type activity.

In a particular embodiment the reduced FX activity is to between 35% and 65% of wild-type activity.

In certain embodiments, the cell has been engineered to possess reduced GDP-keto-6-deoxymannose-3,5-epimerase,4-reductase (FX) activity compared to wild type using gene-editing.

The inventors have discovered that gene engineering (gene-editing) can be employed to generate partial knockouts of a host cell FX gene and such partial FX KO cells produce a ratio, preferably a desired ratio, of afucosylated/fucosylated forms of an expressed protein depending on the degree of FX KO. A cell with a pre-determined degree of partial FX activity can then be selected as the host into which nucleic acid(s) (e.g., one or more polynucleotides) encoding a protein of interest is inserted for expression of the protein by the transfected/transformed host. In this way the protein can be produced with a pre-determined desired or near-desired ratio of fucosylated and afucosylated forms.

Additionally, the inventors have found that Lens culinaris agglutinin (LCA) conjugated to FITC (LCA-FITC) based FACS analysis can be used early during clone screening process to identify clones with varying levels of partial KO of the FX gene (Louie, S.; Haley, B.; Marshall, B.; Heidersbach, A.; Yim, M.; Brozynski, M.; Tang, D.; Lam, C.; Petryniak, B.; Shaw, D.; Shim, J.; Miller, A.; Lowe, J. B.; Snedecor, B.; Misaghi, S., FX knockout CHO hosts can express desired ratios of fucosylated or afucosylated antibodies with high titers and comparable product quality. Biotechnol Bioeng 2017, 114, (3), 632-644; Glinsek, K.; Kramer, L.; Krajnc, A.; Kranjc, E.; Pirher, N.; Marusic, J.; Hellmann, L.; Podobnik, B.; Strukelj, B.; Auslander, D.; Gaber, R., Coupling CRISPR interference with FACS enrichment: New approach in glycoengineering of CHO cell lines for therapeutic glycoprotein production. Biotechnol J 2022, 17, (7), e2100499).

According to a second aspect of the invention there is provided a method of producing a protein, wherein the protein is produced in fucosylated and afucosylated forms in a desired ratio, comprising:
culturing a host cell capable of expressing a protein of interest in fucosylated and afucosylated forms in a near-desired ratio in a culture medium, wherein the host cell has a partial knock-out of the FX gene, and wherein the near-desired ratio of fucosylated to afucosylated forms is adjusted to the desired ratio by increasing or decreasing the amount of fucose in the culture medium.

In particular embodiments of the first or second aspects of the invention, the ratio of fucosylated to afucosylated forms of the expressed protein of interest is determined by the amount of FX activity produced by the cell with partial FX KO.

In a particular embodiment of the first or second aspects of the invention, the host cell has been engineered to possess reduced FX activity compared to wild type by introducing a sequence deletion, a sequence addition or a sequence substitution into the FX gene in the host cell causing a partial knock out of the FX gene. Such cells may be referred to as having a partial knock-out of the FX gene (FXpKO). In specific embodiments, the deletion is in one of the two allele copies of the FX gene.

In particular embodiments, the sequence deletion, sequence addition or sequence substitution to the FX gene has been created using: (a) a clustered, regularly interspaced, short palindromic repeats (CRISPR) system; (b) a transcription activator-like effector nuclease (TALEN) system; (c) a zinc-finger nuclease (ZFN) system; (d) a transposase system; or (e) a meganuclease system.

Particular embodiments pertaining to FX gene editing (engineering) of the host cell are disclosed in the Gene-editing of FX gene section.

In particular embodiments, the host cell is or has been adapted so as to be capable of expressing, or to express, a protein of interest. In a particular embodiment, the cell is or has been adapted to express a protein of interest by transforming or transfecting the cell with one or more nucleic acid molecules capable of expressing the protein of interest.

In certain embodiments, the protein of interest is a heterologous protein or recombinantly produced protein.

In a particular embodiment, the protein of interest is an antibody.

In a particular embodiment, the protein of interest is an Fc-containing protein, e.g., a fusion protein.

In a particular embodiment, the Fc-containing protein comprises an antibody heavy chain or a fragment thereof.

In a particular embodiment, the Fc-containing protein is a full length antibody, in particular wherein the full length antibody is a monoclonal antibody. In certain other embodiments, the Fc-containing protein is a bispecific antibody, a multispecific antibody, or a T cell engager.

Particular embodiments pertaining to the protein of interest are disclosed in the Protein of interest section.

The protein of interest can be expressed from one or more nucleic acid molecules capable of expressing, or that express, the protein of interest. In certain embodiments with respect to a full size antibody, one nucleic acid molecule encodes the heavy chain polypeptide and another nucleic acid molecule encodes the light chain polypeptide. Alternatively, in another specific embodiment, a single nucleic acid molecule encodes both the heavy and light chain polypeptides.

The nucleic acid encoding the protein of interest or a part thereof (e.g., H or L chain polypeptide) will be under the control of a promoter and regulatory elements to effect efficient expression of the protein when in the host cell. The person of skill in the art is able to select and employ a suitable promoter and other regulatory elements to use. Indeed, the ability to clone, transform/transfect and express a protein of interest is within the competence of a person of skill in the art.

In certain embodiments, the nucleic acid(s) encoding the protein of interest are present within one or more vectors, such as a plasmid, that can be transformed or transfected into a host cell using conventional techniques (such as electroporation).

Particular embodiments pertaining to adapting host cells to express a protein of interest are disclosed in the Cells Engineered to Produce a Protein section.

According to the first and second aspects of the invention, the host cell has been engineered to:
(i) produce the protein of interest, and
(ii) possess reduced FX activity compared to wild type.

It will be apparent to the person skilled in the art that modifications (i) and (ii) can be created in either order. As such, a host cell with a normal functioning FX gene can be modified to express a protein of interest (e.g. transfected with nucleic acids suitable for expression of the protein of interest). A transfected host cell that can express the protein of interest (e.g., when cultured in a suitable growth medium under appropriate conditions) can then be selected and engineered to effect a reduction in the FX activity of the cell, e.g. by gene-editing to make a partial knock out of the KX gene. Alternatively, a suitable host cell can be engineered to effect a reduction in the FX activity of the cell, e.g. by gene-editing to make a partial knock out of the KX gene. A modified host cell with a desired level of reduced FX activity can then be selected and adapted (e.g. by transfection) to express the protein of interest.

According to a particular embodiment, the host cell capable of expressing the protein of interest in fucosylated and afucosylated forms in a desired or near-desired ratio is or has been selected from a plurality of cells subjected to partial knock out of FX gene.

According to a particular embodiment, the host cell subjected to gene editing to partially inactivate the FX gene is capable of expressing, or expresses, the protein of interest.

According to a particular embodiment, the host cell subjected to gene editing to partially inactivate the FX gene is further modified to express the protein of interest.

According to a particular embodiment, the host cell subjected to gene editing to partially inactivate the FX gene is further modified to express the protein of interest by transforming or transfecting the gene edited cell with one or more nucleic acid molecules capable of expressing, or expressing, the protein of interest.

As explained herein, the degree of FX activity in the host cell correlates with the amount of fucosylation on the expressed protein. Accordingly, by judicious selection and use of a modified host cell clone that has the desired level of FX activity, it is possible to produce (e.g., express) the protein of interest with a desired or near-desired ratio of fucosylated to afucosylated forms. A desired ratio of fucosylated to afucosylated forms is herein also referred to as a pre-determined ratio of fucosylated to afucosylated forms.

A selected host cell, which has been engineered to (i) produce the protein of interest, and (ii) to possess reduced FX activity (compared to wild type), expresses the protein of interest with the desired ratio of fucosylated to afucosylated forms. However, it may be that the selected host cell, which has been engineered to (i) produce the protein of interest, and (ii) to possess reduced FX activity (compared to wild type), expresses the protein of interest with a near-desired ratio of fucosylated to afucosylated forms. A near-desired ratio is one that is close to but not the actual desired ratio. Thus, for example, if the desired ratio was 70:30 fucosylated:afucosylated forms, a ratio of 65:35 or 75:25 fucosylated:afucosylated forms could represent near-desired ratios.

If the host cell produces the desired protein with a near-desired ratio of fucosylated to afucosylated forms, the ratio can be adjusted to the desired ratio by adjusting the amount of the fucose source in the culture medium so that at the appropriate stage (e.g. end of culturing) the protein is produced with the desired level of fucosylation (that is, the desired ratio of fucosylated to afucosylated forms).

If the production of the desired protein involves culturing the host cell in a suitable medium for a period of time, followed by further culturing for a second period of time in medium to which fucose has been added, the fucose addition day (day that fucose is added to the medium) may be determined in advance. For example, the inventors have found that the desired levels of afucosylated mAb may be approximated depending on the specific productivity (Qp) of the FX KO clone, and fucose addition day (the day fucose is added to the medium since the start of cell culture) can then be experimentally confirmed using small scale production runs.

The specific productivity (Qp) is the amount of antibody that a cell is making/secreting in one day with the units of pg/cell-day. Qp can be determined by measuring the total number of viable cells throughout the duration (days) of production process (e.g. 12-14 days) and dividing the overall titer (in picogram) with that number of cells. Specific productivity dictates how much antibody is made in a day by each cell and that will allow one to determine when the desired % afucosylated antibodies are reached.

However, it may be convenient to determine the level of fucosylation of the expressed protein at one or more time points during the culturing and to then adjust the amount of the fucose source in the culture medium so that the protein is produced with the desired level of fucosylation.

Thus, according to particular embodiments, the method of the first aspect the invention further comprises:
determining the level of fucosylation of the expressed protein; and
adjusting the amount of the fucose source in the culture medium so that the protein is produced with the desired level of fucosylation.

The amount of fucosylation that an expressed protein, or a sample of expressed protein, or a preparation of expressed protein possesses, and/or the ratio of fucosylated to afucosylated forms (level of fucosylation) can be determined by methods as described in Louie, S.; Haley, B.; Marshall, B.; Heidersbach, A.; Yim, M.; Brozynski, M.; Tang, D.; Lam, C.; Petryniak, B.; Shaw, D.; Shim, J.; Miller, A.; Lowe, J. B.; Snedecor, B.; Misaghi, S., FX knockout CHO hosts can express desired ratios of fucosylated or afucosylated antibodies with high titers and comparable product quality. Biotechnol Bioeng 2017, 114, (3), 632-644; and in Glinsek, K.; Kramer, L.; Krajnc, A.; Kranjc, E.; Pirher, N.; Marusic, J.; Hellmann, L.; Podobnik, B.; Strukelj, B.; Auslander, D.; Gaber, R., Coupling CRISPR interference with FACS enrichment: New approach in glycoengineering of CHO cell lines for therapeutic glycoprotein production. Biotechnol J 2022, 17, (7), e2100499.

In some embodiments, determining the level of fucosylation of the protein comprises determining (e.g., level or percentage of) the fucosylated form of the protein. In some embodiments, determining the level of fucosylation of the protein comprises determining the afucosylated form of the protein.

In some embodiments according to (or as applied to) any of the aspects of embodiments above, the fucosylated form of the protein is determined at a glycan structure level. In some embodiments, PNGase F is used to cleave a glycan structure from the protein. In some embodiments, the fucosylated form of the protein is determined at a glycan structure level, wherein the fucosylated form of the protein is determined by capillary electrophoresis (CE).

In some embodiments according to (or as applied to) any of the aspects or embodiments above, the fucosylated form of the protein is determined at a protein level. In some embodiments, the fucosylated form of the protein is determined by mass spectrometry (MS).

In certain embodiments, when culturing the selected host cell that expresses a protein, e.g., under manufacturing conditions, the host cell is cultured in a medium that lacks fucose and after determination of the level of fucosylation of the expressed protein, a source of fucose can be added to the medium to effect an increase in the ratio of fucosylated:afucosylated forms of the protein. In certain embodiments, the host cell that expresses a protein is cultured, e.g., under manufacturing conditions, in a culture medium that lacks fucose, and, after determination of the level of fucosylation of the expressed protein, fucose is added to the medium to effect an increase in the ration of fucosylated:afucosylated forms of the protein.

Alternatively, when culturing the selected host cell that expresses a protein, e.g., under manufacturing conditions, the host cell is cultured in a medium that contains an amount of fucose, and after determination of the level of fucosylation of the expressed protein, culture of the host cell is continued and the amount of fucose in the medium for continued culture can be adjusted so as to effect an increase or decrease in the ratio of fucosylated:afucosylated forms. The addition of fucose will increase the ratio of fucosylated:afucosylated forms produced (increases fucosylation) whereas removal of fucose will effect a decrease in the ratio of fucosylated:afucosylated forms (reduces fucosylation).

In particular embodiments of the first or second aspects of the invention, the culture medium comprises fucose, e.g., a fucose source.

Suitably, the fucose source is a fucose, for example wherein (i) the fucose is L-fucose, (ii) the fucose is L-fucose-1-phosphate, or (iii) the fucose source is GDP-fucose.

In particular embodiments of the first or second aspects of the invention, the host cell is cultured in a fed-batch process, an intensified fed-batch process, a continuous or a semicontinuous process.

In particular embodiments of the first or second aspects of the invention, the fucose source is added to the culture medium via bolus addition, via continuous feed or fed-batch feed.

In particular embodiments of the first or second aspects of the invention, the host cell is a eukaryotic cell, such as a mammalian cell.

In particular embodiments of the first or second aspects of the invention, the host cell is a Chinese Hamster Ovary (CHO) cell. In various embodiments, the CHO cell is a DP12 cell, a DUXB-11 derived DHFR-deficient DP12 cell, a CHO-K1 cell, a DHFR-positive CHO-K1 cell, or a CHOK1M cell.

In some embodiments, the CHO is capable of integrating one or more nucleic acid sequence(s), e.g., polynucleotides, encoding polypeptide chains, or a protein, or fragments of the foregoing, into its genome. In particular embodiments, the integration of nucleic acid sequences encoding one or more polypeptide(s) or protein(s) or fragment(s) thereof is targeted to a particular site in the cell genome (targeted integration) as described in WO 2019/126634, the disclosure of which is hereby incorporated by reference. In other embodiments, the integration of the polypeptide or protein or fragment thereof is random (random integration).

Particular embodiments pertaining to host cells are disclosed in the Host cells section.

In particular embodiments, the method of the first or second aspects of the invention, further comprises isolating and/or purifying the protein from the culture medium.

In particular embodiments, the method of the first or second aspects of the invention that further comprises purifying the protein from the culture medium, further comprises formulating the protein into a pharmaceutical composition comprising a pharmaceutically acceptable excipient or diluent.

The embodiments recited for the first and/or second aspect of the invention can be applied to the other aspects mutatis mutandis.

### Method for Producing a Cell

According to a third aspect, provided herein is a method of producing a cell, e.g., a host cell, e.g., a host cell clone capable of expressing, or expressing, a protein, wherein the protein is produced in fucosylated and afucosylated forms in a desired ratio, comprising:
A.
   (i) subjecting isolated host cells capable of expressing, or expressing, a protein to gene editing to produce individual gene-edited host cell clones with partial knock-out of the FX gene;
   (ii) selecting one or more gene-edited cell clones which produces the protein of interest in fucosylated and afucosylated at the desired ratio; or
B.
   (iii) transfecting a clonal population of isolated host cells with a polynucleotide sequence encoding the protein to produce transfected host cells, wherein said isolated host cells express said protein, wherein said isolated host cells have been gene edited to have reduced FX activity compared to wild type (unmodified) host cells, and wherein the isolated host cells have been selected for a particular level of FX activity that results in the protein being produced in a desired ratio of fucosylated and afucosylated forms; and
   (iv) selecting a transfected host cell that expresses the protein of interest in fucosylated and afucosylated forms at a desired ratio.

In particular embodiments of this aspect of the invention, the method or mode of gene editing is selected from: (a) a clustered, regularly interspaced, short palindromic repeats (CRISPR) system, e.g., a CRISPR/Cas9 system; (b) a transcription activator-like effector nuclease (TALEN) system; (c) a zinc-finger nuclease (ZFN) system; (d) a transposase system; or (e) a meganuclease system.

Particular embodiments pertaining to FX gene editing (engineering) of the host cell are disclosed in the Gene-editing of FX gene section.

In a particular embodiment of this aspect of the invention, the level of fucosylation or the ratio of fucosylated and afucosylated protein produced by a cell clone is determined using LCA-FITC staining and FACS profile analysis.

In particular embodiments, the host cell is a eukaryotic cell, such as a mammalian cell.

In particular embodiments according to the sixth aspect of the invention, the host cell is a Chinese hamster ovary (CHO) cell. In various more specific embodiments, the CHO cell is a DP12 cell, a DUXB-11 derived DHFR-deficient DP12 cell, a CHO-K1 cell, a DHFR-positive CHO-K1 cell, or a CHOK1M cell.

Particular embodiments pertaining to host cells are disclosed in the Host cells section.

Particular embodiments pertaining to adapting host cells to express a protein of interest are disclosed in the Cells engineered to produce a protein section.

The embodiments recited for the first and/or second aspect of the invention can be applied to the third aspect mutatis mutandis.

According to a fourth aspect of the invention there is provided a cell clone capable of expressing a protein of interest produced by the method of the third aspect of the invention.

### Cell Culture

According to a fifth aspect of the invention there is provided a cell culture comprising the host cell clone capable of expressing a protein of interest produced by the method of the third aspect of the invention and a culture medium, the culture medium optionally comprising a source of fucose.

In particular embodiments, the cell culture further comprises afucosylated and fucosylated forms of the protein of interest.

The present application, in some aspects, provides compositions comprising a protein made by any of the methods described herein. In some embodiments, the composition comprises afucosylated and fucosylated forms of the protein at a pre-determined ratio that provides the desired ADCC function.

Particular embodiments pertaining to compositions comprising a protein of interest made according to the present invention are disclosed in the Compositions section.

In some embodiments the composition is a pharmaceutical composition.

Particular embodiments pertaining to pharmaceutical compositions are disclosed in the Pharmaceuticals compositions section.

Process adapted method of producing a protein in fucosylated and afucosylated forms at a desired ratio.

According to a sixth aspect of the invention there is provided method of producing a protein, wherein the protein is produced in fucosylated and afucosylated forms at a desired ratio, comprising
(i) determining the period of time required for culturing a host cell having substantially no FX activity in a culture medium lacking a fucose source prior to culturing said host cell in a culture medium comprising a fucose source sufficient to produce fucosylated and afucosylated forms of the protein in a desired ratio;
(ii) culturing the host cell having substantially no FX activity in a culture medium lacking a fucose source for the period of time determined in step (i); and then
(iii) culturing the host cell in a culture medium comprising a fucose source for a period of time sufficient to produce the protein in fucosylated and afucosylated forms at the desired ratio.

In another embodiment, provided herein is a method of method of producing a protein, wherein the protein is produced in fucosylated and afucosylated forms at a desired ratio, comprising
(i) culturing a host cell that produces the protein, wherein the host cell has substantially no FX activity, in a culture medium lacking a fucose source for a first period of time; and then
(ii) culturing the host cell in a culture medium comprising a fucose source for a second period of time sufficient to produce the protein in fucosylated and afucosylated forms at the desired ratio,
wherein the first period of time is determined prior to step (i), and is determined by (a) culturing the host cell in the same type of culture medium lacking a fucose source for said first period of time, and then (b) culturing said host cell in the culture medium comprising a fucose source for said second period of time, wherein said second period of time is a period of time sufficient to produce fucosylated and afucosylated forms of the protein in a desired ratio.

In a particular embodiment according to the sixth aspect of the invention, the cell is cultured in a fed-batch process, an intensified fed-batch process, a continuous process or a semicontinuous process.

In a particular embodiment according to the sixth aspect of the invention, the culture medium comprising the fucose source is produced by adding a fucose source, e.g., fucose, to the culture medium lacking a fucose source. In another embodiment, the culture medium comprising the fucose source is a different batch of the same type of medium as the culture medium without a fucose source, to which fucose has been added.

In a particular embodiment according to the sixth aspect of the invention, the fucose source is added, e.g., to the cell culture medium lacking a fucose source, via bolus addition.

In a particular embodiment according to the sixth aspect of the invention, the fucose source is added via continuous feed or fed-batch feed. In a specific embodiment, the fucose source is added via continuous feed or fed-batch feed so as to maintain the concentration of fucose in the culture medium at about 1 mM.

In a particular embodiment according to the sixth aspect of the invention, the amount of the added fucose source in the culture medium is a sufficient amount to bring the final concentration of fucose in the cell culture medium to between 0.01 mM and 10 mM.

In various embodiments, the fucose source is a fucose, in particular wherein (i) the fucose is L-fucose, (ii) the fucose is L-fucose-1-phosphate, or (iii) the fucose source is GDP-fucose.

In particular embodiments according to the sixth aspect of the invention, the protein is an Fc-containing protein or a fusion protein. In certain embodiments, the Fc-containing protein comprises an antibody heavy chain or a fragment thereof or a fusion protein. Suitably, the Fc-containing protein is a full length antibody, in particular wherein the full length antibody is a monoclonal antibody, a bispecific- or a trispecific-antibody, or a complex antibody. In other embodiments, the Fc-containing protein is a T cell engager.

In particular embodiments according to the sixth aspect of the invention, the host cell is a eukaryotic cell, such as a mammalian cell. Particular embodiments pertaining to the host cell are disclosed in the Host cells section.

In particular embodiments according to the sixth aspect of the invention, the host cell is a Chinese hamster ovary (CHO) cell. Suitably, the CHO cell is selected from the group consisting of a DP12 cell, a DUXB-11 derived DHFR-deficient DP12 cell, a CHO-K1 cell, a DHFR-positive CHO-K1 cell, and a CHOK1M cell.

In particular embodiments according to the sixth aspect of the invention, the host cell has substantially no FX activity due to the FX gene in the host cell having been knocked out by a sequence deletion or by a sequence addition or substitution.

In particular embodiments according to the sixth aspect of the invention, the FX gene has been knocked out using: (a) a clustered, regularly interspaced, short palindromic repeats (CRISPR) system; (b) a transcription activator-like effector nuclease (TALEN) system; (c) a zinc-finger nuclease (ZFN) system; (d) a transposase system; or (e) a meganuclease system.

Particular embodiments pertaining to FX gene editing (engineering) of the host cell are disclosed in the Gene-editing of FX gene section.

In particular embodiments according to the sixth aspect of the invention, the host cell having substantially no FX activity is cultured in the culture medium lacking a fucose source for up to 14 days, such as 1, 2, 3, 4, 5, 6, 7, 8, 9, 10, 11, 12, 13 or 14 days, and then cultured in the culture medium comprising a fucose source for up to 14 days, such as 1, 2, 3, 4, 5, 6, 7, 8, 9, 10, 11, 12, 13 or 14 days.

In particular embodiments according to the sixth aspect of the invention, the initiation of the culturing of the host cell having substantially no FX activity in the culture medium comprising a fucose source occurs between 1 and 14 days after initiation of the culturing of the host cell in the culture medium lacking a fucose source.

In certain embodiments, the initiation of the culturing of the host cell having substantially no FX activity in the culture medium comprising a fucose source occurs 1, 2, 3, 4, 5, 6, 7, 8, 9, 10, 11, 12, 13 or 14 days after initiation of the culturing of the host cell in the culture medium lacking a fucose source, or between 1 and 4 days, 2 and 5 days, 3 and 6 days, 4 and 7 days, 5 and 8 days, 6 and 9 days, 7 and 10 days, 8 and 11 days, 9 and 12 days, 10 and 13 days, or 11 and 14 days after initiation of the culturing of the host cell in a culture medium lacking a fucose source.

In a particular embodiment according to the sixth aspect of the invention, the method further comprises purifying the protein from the culture medium.

In a particular embodiment according to the sixth aspect of the invention, the method further comprises purifying the protein from the culture medium and formulating the purified protein into a pharmaceutical composition comprising a pharmaceutically acceptable excipient or diluent.

Particular embodiments pertaining to pharmaceutical composition are disclosed in the Pharmaceuticals compositions section.

### Purification

Methods described herein, in some aspects, further comprise methods for isolating a protein produced by the methods described herein.

Methods for isolating or purifying a protein are well known in the art. See, e.g., Huse et al., J Biochem Bioph Meth, 51, 2002. Proteins can be produced intracellularly or directly secreted into the medium. If the proteins are produced intracellularly, as a first step, the particulate debris, either host cells or lysed fragments, are removed, for example, by centrifugation or ultrafiltration. If the proteins are secreted into the medium, supernatants from such expression systems are generally first concentrated using a commercially available protein concentration filter, for example, an Amicon or Millipore PELLICON® ultrafiltration unit. In some embodiments, a protease inhibitor may be included in any of the foregoing steps to inhibit proteolysis and antibiotics may be included to prevent the growth of adventitious contaminants.

The protein within the composition prepared from the cells, in particular an Fc-containing protein, can be purified using, for example, hydroxyapatite chromatography, gel electrophoresis, dialysis, and affinity chromatography, with affinity chromatography being the preferred purification technique. For Fc-containing proteins, the suitability of protein A as an affinity ligand depends on the species and isotype of any immunoglobulin Fc domain that is present in the Fc-containing protein. Protein A can be used to purify Fc-containing proteins that are based on human immunoglobulins containing IgG1, IgG2, or IgG4 heavy chains (See, e.g., Lindmark et al., J Immunol Meth, 62, 1983). Protein G is recommended for all mouse isotypes and for human IgG3 (See, e.g., Guss et al., EMBO, 5, 1986).

The matrix to which the affinity ligand is attached is often agarose, but other matrices are available. Mechanically stable matrices such as controlled pore glass or poly(styrene-divinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein to be purified.

In some embodiments, a method of purifying the protein expressed from the host cell, according to the methods of the invention, comprises using a filter. In some embodiments, the filter is a diafiltration system. In some embodiments, the filter is an ultrafiltration system. In some embodiments, the filter is a viral filtration system. In some embodiments, the purifying of a protein comprises using a series of filtration steps. In some embodiments, the series of filtration steps is selected from at least one of the following: diafiltration, ultrafiltration, and viral filtration.

In some embodiments, provided herein is a method of purifying the protein expressed from the host cell according to the methods of the invention comprising using a series of protein purification techniques selected from one or more of filtration, protein A purification, cation exchange purification, strong cation exchange purification, anion exchange purification, reverse phase purification, and multimodal or mixed-mode purification.

The protein produced by the methods of the invention may be formulated into a pharmaceutical composition. See also Pharmaceutical compositions section.

### Predetermined or Desired Ratio of Fucosylated and Afucosylated Forms

The methods of the invention can produce a preparation of protein with a desired ratio of fucosylated to afucosylated forms. The desired ratio of fucosylated to afucosylated forms may be referred to herein as a predetermined ratio of fucosylated to afucosylated forms.

In some embodiments, the predetermined ratio of fucosylated and afucosylated forms of a protein is about 50:1 to about 1:50, about 50:2.5 to about 1:50, about 50:5.5 to about 1:50, about 50:8.5 to about 1:50, about 50:12.5 to about 1:50, about 50:15.5 to about 1:50, about 50:21.5 to about 1:50, about 50:27 to about 1:50, about 50:33.5 to about 1:50, about 50:41 to about 1:50, about 50:50 to about 1:50, about 50:61 to about 1:50, about 50:75 to about 1:50, about 50:93 to about 1:50, about 50:116.5 to about 1:50, about 50:150 to about 1:50, about 50:200 to about 1:50, about 50:283.5 to about 1:50, about 50:450 to about 1:50, or about 50:950 to about 1:50.

In some embodiments, the predetermined ratio of fucosylated and afucosylated forms of a protein is about 50:1 to about 2.5:50, about 50:1 to about 5.5:50, about 50:1 to about 8.5:50, about 50:1 to about 12.5:50, about 50:1 to about 15.5:50, about 50:1 to about 21.5:50, about 50:1 to about 27:50, about 50:1 to about 33.5:50, about 50:1 to about 41:50, about 50:1 to about 50:50, about 50:1 to about 61:50, about 50:1 to about 75:50, about 50:1 to about 93:50, about 50:1 to about 116.5:50, about 50:1 to about 150:50, about 50:1 to about 200:50, about 50:1 to about 283.5:50, about 50:1 to about 450:50, or about 50:1 to about 950:50.

In some embodiments, the predetermined ratio of fucosylated and afucosylated forms of a protein is about 50:2.5 to about 2.5:50, about 50:5.5 to about 5.5:50, about 50:8.5 to about 8.5:50, about 50:12.5 to about 12.5:50, about 50:15.5 to about 15.5:50, about 50:15.5 to about 21.5:50, about 50:27 to about 27:50, about 50:33.5 to about 33.5:50, or about 50:41 to about 41:50. In some embodiments, the predetermined ratio of fucosylated and afucosylated forms of a protein is about 12.5:50 to about 2.5:50, about 12.5:50 to about 5.5:50, about 12.5:50 to about 8.5:50, about 12.5:50 to about 12.5:50, or about 12.5:50 to about 15.5:50.

In some embodiments, the predetermined ratio of fucosylated and afucosylated forms of a protein is about 21.5:50 to about 2.5:50, about 21.5:50 to about 5.5:50, about 21.5:50 to about 8.5:50, about 21.5:50 to about 12.5:50, or about 21.5:50 to about 15.5:50.

In some embodiments, the predetermined ratio of fucosylated and afucosylated forms of a protein is about 33.5:50 to about 2.5:50, about 33.5:50 to about 5.5:50, about 33.5:50 to about 8.5:50, about 33.5:50 to about 12.5:50, about 33.5:50 to about 15.5:50, about 33.5:50 to about 21.5:50, or about 33.5:50 to about 27:50.

In some embodiments, the predetermined ratio of fucosylated and afucosylated forms of a protein is about 50:50 to about 2.5:50, about 50:50 to about 5.5:50, about 50:50 to about 8.5:50, about 50:50 to about 12.5:50, about 50:50 to about 15.5:50, about 50:50 to about 21.5:50, about 50:50 to about 27:50, about 50:50 to about 33.5:50, or about 50:50 to about 41:50.

In some embodiments, the predetermined ratio of fucosylated and afucosylated forms of a protein is about 75:50 to about 2.5:50, about 75:50 to about 5.5:50, about 75:50 to about 8.5:50, about 75:50 to about 12.5:50, about 75:50 to about 15.5:50, about 75:50 to about 21.5:50, about 75:50 to about 27:50, about 75:50 to about 33.5:50, about 75:50 to about 41:50, about 75:50 to about 50:50, or about 75:50 to about 61:50.

In some embodiments, the predetermined ratio of fucosylated and afucosylated forms of a protein is about 116.5:50 to about 2.5:50, about 116.5:50 to about 5.5:50, about 116.5:50 to about 8.5:50, about 116.5:50 to about 12.5:50, about 116.5:50 to about 15.5:50, about 116.5:50 to about 21.5:50, about 116.5:50 to about 27:50, about 116.5:50 to about 33.5:50, about 116.5:50 to about 41:50, about 116.5:50 to about 50:50, about 116.5:50 to about 61:50, about 116.5:50 to about 75:50, or about 116.5:50 to about 93:50.

In some embodiments, the predetermined ratio of fucosylated and afucosylated forms of a protein is about 200:50 to about 2.5:50, about 200:50 to about 5.5:50, about 200:50 to about 8.5:50, about 200:50 to about 12.5:50, about 200:50 to about 15.5:50, about 200:50 to about 21.5:50, about 200:50 to about 27:50, about 200:50 to about 33.5:50, about 200:50 to about 41:50, about 200:50 to about 50:50, about 200:50 to about 61:50, about 200:50 to about 75:50, about 200:50 to about 93:50, about 200:50 to about 116.5:50, or about 200:50 to about 150:50.

In some embodiments, the predetermined ratio of fucosylated and afucosylated forms of a protein is about 50:1, about 50:2.5, about 50:5.5, about 50:8.5, about 50:12.5, about 50:15.5, about 50:21.5, about 50:27, about 50:33.5, about 50:41, about 50:50, about 50:61, about 50:75, about 50:93, about 50:116.5, about 50:150, about 50:200, about 50:283.5, about 50:450, or about 50:950.

The amount of a fucose source in a culture medium necessary to “fine tune” production of the desired ratio of fucosylated and afucosylated forms of the protein will be influenced by and dependent on various components of the system, such as the type of host cell, number of host cells, protein production rate, type of fucose source, and the predetermined ratio of fucosylated and afucosylated forms of a protein desired, however, the amount of fucose source to add can be determined empirically by a person of skill in the art, or the fucose can be added serially in small amounts and the effect on the ratio of fucosylated and afucosylated forms of a protein measured until the desired ratio is obtained.

Various sources of fucose could be used in the methods of the invention requiring addition of a fucose source to the culture medium. In some embodiments, the fucose source added to the culture medium is sufficient to bring the concentration of fucose to between about 0.01 mM and about 1 mM.

In some embodiments, the fucose is L-fucose. In some embodiments, the fucose is L-fucose-1-phosphate. In some embodiments, the fucose source is GDP-fucose.

The amount of a fucose source in a culture medium comprising the fucose source in an amount sufficient to produce the fucosylated and afucosylated forms of the protein at the predetermined ratio is largely dependent on components of the system, such as the type of host cell, number of host cells, type of fucose source, protein production rate, and the predetermined ratio of fucosylated and afucosylated forms of a protein. In some embodiments, the fucose source added to the culture medium is in an amount sufficient to bring the concentration of fucose to between about 0.01 mM and about 1 mM. In some embodiments, the fucose source is added to the culture medium is in an amount sufficient to bring the concentration of fucose to between about 0.01 mM and about 0.1 mM, about 0.01 mM and about 0.09 mM, about 0.01 mM and about 0.08 mM, about 0.01 mM and about 0.07 mM, about 0.01 mM and about 0.06 mM, about 0.01 mM and about 0.05 mM, about 0.01 mM and about 0.04 mM, about 0.01 mM and about 0.03 mM, about 0.01 mM and about 0.02 mM, about 0.02 mM and about 0.1 mM, about 0.02 mM and about 0.09 mM, about 0.02 mM and about 0.08 mM, about 0.02 mM and about 0.08 mM, about 0.02 mM and about 0.07 mM, about 0.02 mM and about 0.06 mM, about 0.02 mM and about 0.05 mM, about 0.02 mM and about 0.04 mM, about 0.02 mM and about 0.03 mM, about 0.03 mM and about 0.1 mM, about 0.03 mM and about 0.09 mM, about 0.03 mM and about 0.08 mM, about 0.03 mM and about 0.07 mM, about 0.03 mM and about 0.06 mM, about 0.03 mM and about 0.05 mM, about 0.03 mM and about 0.04 mM, about 0.04 mM and about 0.1 mM, about 0.04 mM and about 0.09 mM, about 0.04 mM and about 0.08 mM, about 0.04 mM and about 0.07 mM, about 0.04 mM and about 0.6 mM, about 0.04 mM and about 0.05 mM, about 0.05 mM and about 0.1 mM, about 0.05 mM and about 0.09 mM, about 0.05 mM and about 0.08 mM, about 0.05 mM and about 0.07 mM, about 0.05 mM and about 0.06 mM, about 0.06 mM and about 0.1 mM, about 0.06 mM and about 0.09 mM, about 0.06 mM and about 0.08 mM, about 0.06 mM and about 0.07 mM, about 0.07 mM and about 0.1 mM, about 0.07 mM and about 0.09 mM, about 0.07 mM and about 0.08 mM, about 0.08 mM and about 0.1 mM, about 0.08 mM and about 0.9 mM, or about 0.09 mM and about 0.1 mM. In some embodiments, the fucose source is about 0.01 mM, about 0.02 mM, about 0.03 mM, about 0.04 mM, about 0.05 mM, about 0.06 mM, about 0.07 mM, about 0.08 mM, about 0.09 mM, about 0.1 mM, about 0.11 mM, about 0.12 mM, about 0.13 mM, about 0.14 mM, about 0.15 mM, about 0.16 mM, about 0.17 mM, about 0.18 mM, about 0.19 mM, about 0.2 mM, about 0.3 mM, about 0.4 mM, about 0.5 mM, about 0.6 mM, about 0.7 mM, about 0.8 mM, about 0.9 mM, or about 1 mM.

### Gene-Editing of FX Gene

In some embodiments, the FX gene in a host cell is fully (including substantially fully) or partially inactivated. As used herein, “inactivated” refers to inhibiting the translation, or potential future translation, of a functional gene (i.e., expression of a functional enzyme). Inactivation can occur at any stage or process of gene expression, including, but not limited to, transcription, translation, and protein expression, and inactivation can affect any gene or gene product including, but not limited to, DNA, RNA, such as mRNA, and polypeptides.

In some embodiments, the FX gene in the host cell is inactivated, wherein FX activity is based on a DNA level (e.g., detectable amount of FX gene DNA). In some embodiments, the FX gene in the host cell is inactivated, wherein FX activity is based on a RNA level (e.g., detectable amount of FX gene RNA). In some embodiments, the FX gene in the host cell is inactivated, wherein FX activity is based on a polypeptide level (e.g., detectable amount of FX protein).

Provided in the present application are also methods of producing a knockout host cell. For example, methods include, but are not limited to use of CRISPR, TALEN, ZFN, and meganuclease systems. In some embodiments, the host cell comprises a gene deletion or gene addition or substitution.

Generally, the methods of producing a host cell comprising partial FX activity comprise partially inactivating the FX gene of the host cell and the methods of producing a host cell comprising full FX activity comprise fully inactivating the FX gene of the host cell. Methods and techniques for partially or fully/completely inactivating the FX gene in a host cell include, but are not limited to, the use of RNA inhibition, such as small interfering RNA (siRNA), small hairpin RNA (shRNA; also referred to as a short hairpin RNA), or antisense oligonucleotide, or clustered, regularly interspaced, short palindromic repeats (CRISPR), e.g., a CRISPR/Cas9 system, or transcription activator-like effector nuclease (TALEN), or zinc-finger nuclease (ZFN), or homologous recombination, or non-homologous end-joining, or meganuclease, or enzyme inhibition. See, e.g., O'Keefe, Mater. Methods, 3, 2013; Doench et al., Nat Biotechnol, 32, 2014; Gaj et al., Trends Biotechnol, 31, 2014; and Silva et al., Curr Gene Ther, 11, 2011.

### CRISPR (Clustered Regularly Interspaced Short Palindromic Repeats)

Generally, the CRISPR system used herein can comprise a caspase protein, such as Cas9, and an RNA sequence comprising a nucleotide sequence, referred to as a guide sequence, that is complementary to a sequence of interest. The caspase and RNA sequence form a complex that identify a DNA sequence of a host cell, and subsequently the nuclease activity of the caspase allows for cleavage of the DNA strand. Caspases isotypes have single-stranded DNA or double-stranded DNA nuclease activity. Design of guide RNA sequences and number of guide RNA sequences used in a CRISPR system allow for removal of a specific sequence of a gene and/or addition of a DNA sequence.

In some embodiments, there is provided a method of producing a host cell, wherein the host cell comprises substantially no FX activity or partial FX activity, comprising inactivating the FX gene using a CRISPR system. In some embodiments, provided herein is a method of producing a host cell comprising inactivating the FX gene using a CRISPR system comprising a coding vector. In some embodiments, provided herein is a method of producing a host cell comprising inactivating the FX gene using a CRISPR system comprising a coding vector comprising a DNA endonuclease gene. In some embodiments, provided herein is a method of producing a host cell comprising inactivating the FX gene using a CRISPR system comprising a coding vector comprising a CAS gene. In some embodiments, provided herein is a method of producing a host cell comprising inactivating the FX gene using a CRISPR system comprising a coding vector comprising a CAS9 gene. In some embodiments, provided herein is a method of producing a host cell comprising inactivating the FX gene using a CRISPR system comprising a coding vector encoding a CAS9 gene. In some embodiments, provided herein is a method of producing a host cell comprising inactivating the FX gene using a CRISPR system comprising a Cas protein. In some embodiments, provided herein is a method of producing a host cell comprising inactivating the FX gene using a CRISPR system comprising a Cas9 protein. In some embodiments, provided herein is a method of producing a host cell comprising inactivating a FX gene using a CRISPR system comprising a coding vector encoding a RNA molecule capable of interacting with the Cas9 protein. In some embodiments, provided herein is a method of producing a host cell comprising inactivating the FX gene using a CRISPR system comprising a coding vector encoding a RNA molecule comprising a guide RNA (gRNA) unit, wherein the gRNA unit comprises a nucleotide sequence that is complementary to a portion of a FX gene sequence. In some embodiments, provided herein is a method of producing a host cell comprising inactivating the FX gene using a CRISPR system comprising a RNA molecule comprising a gRNA unit, wherein the gRNA unit comprises a nucleotide sequence that is complementary to a portion of a FX gene sequence. In some embodiments, provided herein is a method of producing a host cell comprising inactivating the FX gene using a CRISPR system comprising a coding vector encoding a RNA molecule comprising a trans-activating crRNA (tracrRNA) unit. In some embodiments, provided herein is a method of producing a host cell comprising inactivating the FX gene using a CRISPR system comprising a RNA molecule comprising a tracrRNA unit. In some embodiments, provided herein is a method of producing a host cell comprising inactivating the FX gene using a CRISPR system comprising a coding vector encoding a RNA molecule comprising a gRNA unit and a tracrRNA unit, wherein the gRNA unit comprises a nucleotide sequence that is complementary to a portion of a gene sequence. In some embodiments, provided herein is a method of producing a host cell comprising inactivating the FX gene using a CRISPR system comprising a RNA molecule comprising a gRNA unit and a tracrRNA unit, wherein the gRNA unit comprises a nucleotide sequence that is complementary to a portion of a FX gene sequence. In some embodiments, provided herein is a method of producing a host cell comprising inactivating the FX gene using a CRISPR system comprising: a) a first RNA molecule comprising a gRNA unit, wherein the gRNA unit comprises a first nucleotide sequence that is complementary to a portion of a FX gene sequence; and b) a second RNA molecule comprising a gRNA unit, wherein the gRNA unit comprises a second nucleotide sequence that is complementary to a portion of a FX gene sequence. In some embodiments, the first nucleotide sequence and second nucleotide sequence are different. In some embodiments, the first nucleotide sequence is complementary to a portion of a FX gene sequence that is in a different location than the region of the portion of the FX gene that is complementary to the second nucleotide sequence. In some embodiments a host cell wherein the FX gene has been inactivated has no functional FX activity. Such host cell may be referred to as a full FX knockout cell. In another embodiment a host cell wherein the FX gene has been inactivated has partial FX activity. Such host cell may be referred to as a partial FX knockout cell.

In some embodiments, provided herein is a method of producing a host cell comprising delivering a CRISPR system to the host cell. In some embodiments, provided herein is a method of producing a host cell comprising delivering to the host cell a vector comprising a CRISPR system using a delivery vector. In some embodiments, the delivery vector is a virus vector. In some embodiments, the delivery vector is a lentivirus. In some embodiments, the delivery vector is an adenovirus. In some embodiments, the vector comprises a promoter.

### TALENs (Transcription Activator-Like Effector Nucleases)

Generally, the TALEN system used herein can comprise one or more restriction nucleases and two or more protein complexes that allow for recognition of a DNA sequence and subsequent double-stranded DNA cleavage. A protein complex of the TALEN system comprises a number of transcription activator-like effectors (TALEs), each recognizing a specific nucleotide, and a domain of a restriction nuclease. Generally, a TALEN system is designed so that two protein complexes, each comprising TALEs and a domain of a restriction nuclease, will individually bind to DNA sequences in a manner to allow for the two domains (one from each protein complex) of a restriction nuclease to form an active nuclease and cleave a specific DNA sequence. Design of number of protein complexes and sequences to be cleaved in a TALEN system allows for removal of a specific stretch of a gene and/or addition of a DNA sequence.

In some embodiments, provided herein is a method of producing a host cell, wherein the host cell has a reduced level of FX activity, comprising inactivating the FX gene using a TALEN system. In some embodiments, provided herein is a method of producing a host cell comprising inactivating the FX gene using a TALEN system comprising a first TALEN unit. In some embodiments, the first TALEN unit comprises a first TALEN binding unit. In some embodiments, the first TALEN binding unit comprises at least one transcription activator-like effector (TALE) and a first nuclease domain. In some embodiments, the first TALEN binding unit comprises at least 3, 4, 5, 6, 7, 8, 9, 10, 11, 12, 13, 14, or 15 TALEs and a first nuclease domain, wherein the TALEs are linked together, and wherein the linked TALEs recognize a portion of a FX nucleotide sequence. In some embodiments, the first TALEN binding unit comprises at least 3, 4, 5, 6, 7, 8, 9, 10, 11, 12, 13, 14, or 15 TALEs and a first nuclease domain, wherein the TALEs are linked together, wherein the linked TALEs recognize a portion of a FX nucleotide sequence, and wherein the linked TALEs are further linked to the first nuclease domain. In some embodiments, the first TALEN unit further comprises a second TALEN binding unit. In some embodiments, the second TALEN binding unit comprises at least one transcription activator-like effector (TALE) and a second nuclease domain. In some embodiments, the second TALEN binding unit comprises at least 3, 4, 5, 6, 7, 8, 9, 10, 11, 12, 13, 14, or 15 TALEs and a second nuclease domain, wherein the TALEs are linked together, and wherein the linked TALEs recognize a portion of a FX nucleotide sequence. In some embodiments, the second TALEN binding unit comprises at least 3, 4, 5, 6, 7, 8, 9, 10, 11, 12, 13, 14, or 15 TALEs and a second nuclease domain, wherein the TALEs are linked together, wherein the linked TALEs recognize a portion of a FX nucleotide sequence, and wherein the linked TALEs are further linked to the second nuclease domain. In some embodiments, the first TALEN binding unit and second TALEN binding unit bind to different sequences of the FX gene. In some embodiments, the first nuclease domain is a domain of an endonuclease. In some embodiments, the first nuclease domain is a domain of a restriction endonuclease. In some embodiments, the first nuclease domain is a domain of Fokl. In some embodiments, the second nuclease domain is a domain of an endonuclease. In some embodiments, the second nuclease domain is a domain of a restriction endonuclease. In some embodiments, the second nuclease domain is a domain of Fokl. In some embodiments, the first nuclease domain and second nuclease domain associate to comprise an active restriction endonuclease. In some embodiments, the first nuclease domain and second nuclease domain associate to comprise an active Fokl enzyme.

In some embodiments, there is provided a method of producing a host cell comprising inactivating the FX gene using a TALEN system further comprising a second TALEN unit. In some embodiments, the second TALEN unit comprises a third TALEN binding unit. In some embodiments, the third TALEN binding unit comprises at least one transcription activator-like effector (TALE) and a third nuclease domain. In some embodiments, the third TALEN binding unit comprises at least 3, 4, 5, 6, 7, 8, 9, 10, 11, 12, 13, 14, or 15 TALEs and a third nuclease domain, wherein the TALEs are linked together, and wherein the linked TALEs recognize a portion of a FX nucleotide sequence. In some embodiments, the third TALEN binding unit comprises at least 3, 4, 5, 6, 7, 8, 9, 10, 11, 12, 13, 14, or 15 TALEs and a third nuclease domain, wherein the TALEs are linked together, wherein the linked TALEs recognize a portion of a FX nucleotide sequence, and wherein the linked TALEs are further linked to the third nuclease domain. In some embodiments, the second TALEN unit further comprises a fourth TALEN binding unit. In some embodiments, the fourth TALEN binding unit comprises at least one transcription activator-like effector (TALE) and a fourth nuclease domain. In some embodiments, the fourth TALEN binding unit comprises at least 3, 4, 5, 6, 7, 8, 9, 10, 11, 12, 13, 14, or 15 TALEs and a fourth nuclease domain, wherein the TALEs are linked together, and wherein the linked TALEs recognize a portion of a FX nucleotide sequence. In some embodiments, the fourth TALEN binding unit comprises at least 3, 4, 5, 6, 7, 8, 9, 10, 11, 12, 13, 14, or 15 TALEs and a fourth nuclease domain, wherein the TALEs are linked together, wherein the linked TALEs recognize a portion of a FX nucleotide sequence, and wherein the linked TALEs are further linked to the fourth nuclease domain. In some embodiments, the third TALEN binding unit and fourth TALEN binding unit bind to different sequences of the FX gene. In some embodiments, the third nuclease domain is a domain of an endonuclease. In some embodiments, the third nuclease domain is a domain of a restriction endonuclease. In some embodiments, the third nuclease domain is a domain of Fokl. In some embodiments, the fourth nuclease domain is a domain of an endonuclease. In some embodiments, the fourth nuclease domain is a domain of a restriction endonuclease. In some embodiments, the fourth nuclease domain is a domain of Fokl. In some embodiments, the third nuclease domain and fourth nuclease domain associate to comprise an active restriction endonuclease. In some embodiments, the third nuclease domain and fourth nuclease domain associate to comprise an active Fokl enzyme.

In some embodiments, provided herein is a method of producing a host cell, wherein the host cell has substantially no FX activity or partial FX activity, comprising fully or partially inactivating the FX gene using a TALEN system, wherein the TALEN system comprises a first TALEN unit and a second TALEN unit that bind to different, non-overlapping portions of a FX gene sequence.

In some embodiments, provided herein is a method of producing a host cell, wherein the host cell has substantially no FX activity or partial FX activity, comprising fully or partially inactivating the FX gene using a TALEN system, wherein the TALEN system comprises a coding vector encoding a first TALEN unit. In some embodiments, provided herein is a method of producing a host cell, wherein the host cell has substantially no FX activity or partial FX activity, comprising fully or partially inactivating the FX gene using a TALEN system, wherein the TALEN system comprises a coding vector encoding a first TALEN unit, wherein the TALEN system comprises a coding vector encoding a second TALEN unit. In some embodiments, provided herein is a method of producing a host cell, wherein the host cell has substantially no FX activity or partial FX activity, comprising fully or partially inactivating the FX gene using a TALEN system, wherein the TALEN system comprises a coding vector encoding a first TALEN unit, wherein the TALEN system comprises a coding vector encoding a first TALEN unit and a second TALEN unit.

In some embodiments, provided herein is a method of producing a host cell, wherein the host cell has substantially no FX activity or partial FX activity, comprising fully or partially inactivating the FX gene using a TALEN system, wherein the TALEN system comprises a first TALEN unit. In some embodiments, provided herein is a method of producing a host cell, wherein the host cell has substantially no FX activity or partial FX activity, comprising fully or partially inactivating the FX gene using a TALEN system, wherein the TALEN system comprises a second TALEN unit. In some embodiments, provided herein is a method of producing a host cell, wherein the host cell has substantially no FX activity or partial FX activity, comprising fully or partially inactivating the FX gene using a TALEN system, wherein the TALEN system comprises a first TALEN unit and a second TALEN unit.

In some embodiments, the first TALEN binding unit comprises a group of linked TALEs, wherein the group of TALEs recognize a nucleotide sequence. In some embodiments, the nucleotide sequence is a sequence comprising a portion of a FX gene. In some embodiments, the nucleotide sequence is a sequence comprising a portion of a FX gene promoter. In some embodiments, the nucleotide sequence is a sequence comprising a portion of a sequence flanking a FX gene. In some embodiments, the sequence is at least about 80%, at least about 85%, at least about 90%, at least about 95%, or 100% homologous to a portion of a FX gene. In some embodiments, the sequence is at least about 80%, at least about 85%, at least about 90%, at least about 95%, or 100% homologous to a portion of a FX gene promoter. In some embodiments, the sequence is at least about 80%, at least about 85%, at least about 90%, at least about 95%, or 100% homologous to a portion of a sequence flanking a FX gene.

In some embodiments, provided herein is a method of producing a host cell comprising delivering a TALEN system to the host cell. In some embodiments, there is provided a method of producing a host cell comprising delivering a vector comprising a TALEN system using a delivery vector. In some embodiments, the delivery vector is a virus vector. In some embodiments, the delivery vector is a lentivirus. In some embodiments, the delivery vector is an adenovirus.

In some embodiments provided herein is a host cell wherein the FX gene has been inactivated has no functional FX activity. Such host cell may be referred to as a full FX knockout cell. In another embodiment a host cell wherein the FX gene has been inactivated has partial FX activity. Such host cell may be referred to as a partial FX knockout cell.

### Zinc Finger (ZFN)

Generally, the ZFN system used herein can comprise one or more restriction nucleases and two or more protein complexes that allow for recognition of a DNA sequence and subsequent double-stranded DNA cleavage. A protein complex of the ZFN system comprises a number of zinc fingers, each recognizing a specific nucleotide codon, and a domain of a restriction nuclease. Generally, a ZFN system is designed so that two protein complexes, each comprising zinc fingers and a domain of a restriction nuclease, will individually bind to DNA sequences in a manner to allow for the two domains (one from each protein complex) of a restriction nuclease to form an active nuclease and cleave a specific DNA sequence. Design of number of protein complexes and sequences to be cleaved in a ZFN system allows for removal of a specific stretch of a gene and/or addition of a DNA sequence.

In some embodiments, provided herein is a method of producing a host cell, wherein the host cell has substantially no FX activity or partial FX activity, comprising inactivating the FX gene using a ZFN system. In some embodiments, provided herein is a method of producing a host cell comprising inactivating the FX gene using a ZFN system comprising a first ZFN unit. In some embodiments, the first ZFN unit comprises a first ZFN binding unit. In some embodiments, the first ZFN binding unit comprises at least one zinc finger and a first nuclease domain. In some embodiments, the first ZFN binding unit comprises at least 2, 3, 4, 5, 6, 7, 8, 9, or 10 zinc fingers and a first nuclease domain, wherein the zinc fingers are linked together, and wherein the linked zinc fingers recognize a portion of a FX nucleotide sequence. In some embodiments, the first ZFN binding unit comprises at least 2, 3, 4, 5, 6, 7, 8, 9, or 10 zinc fingers and a first nuclease domain, wherein the zinc fingers are linked together, wherein the linked zinc fingers recognize a portion of a FX nucleotide sequence, and wherein the linked zinc fingers are further linked to the first nuclease domain. In some embodiments, the first ZFN unit further comprises a second ZFN binding unit. In some embodiments, the second ZFN binding unit comprises at least one zinc finger and a second nuclease domain. In some embodiments, the second ZFN binding unit comprises at least 2, 3, 4, 5, 6, 7, 8, 9, or 10 zinc fingers and a second nuclease domain, wherein the zinc fingers are linked together, and wherein the linked zinc fingers recognize a portion of a FX nucleotide sequence. In some embodiments, the second ZFN binding unit comprises at least 2, 3, 4, 5, 6, 7, 8, 9, or 10 zinc fingers and a second nuclease domain, wherein the zinc fingers are linked together, wherein the linked zinc fingers recognize a portion of a FX nucleotide sequence, and wherein the linked zinc fingers are further linked to the second nuclease domain. In some embodiments, the first ZFN binding unit and second ZFN binding unit bind to different sequences of the FX gene. In some embodiments, the first nuclease domain is a domain of an endonuclease. In some embodiments, the first nuclease domain is a domain of a restriction endonuclease. In some embodiments, the first nuclease domain is a domain of Fokl. In some embodiments, the second nuclease domain is a domain of an endonuclease. In some embodiments, the second nuclease domain is a domain of a restriction endonuclease. In some embodiments, the second nuclease domain is a domain of Fokl. In some embodiments, the first nuclease domain and second nuclease domain associate to comprise an active restriction endonuclease. In some embodiments, the first nuclease domain and second nuclease domain associate to comprise an active Fokl enzyme.

In some embodiments, provided herein is a method of producing a host cell comprising inactivating the FX gene using a ZFN system further comprising a second ZFN unit. In some embodiments, the second ZFN unit comprises a third ZFN binding unit. In some embodiments, the third ZFN binding unit comprises at least one zinc finger and a third nuclease domain. In some embodiments, the third ZFN binding unit comprises at least 2, 3, 4, 5, 6, 7, 8, 9, or 10 zinc fingers and a third nuclease domain, wherein the zinc fingers are linked together, and wherein the linked zinc fingers recognize a portion of a FX nucleotide sequence. In some embodiments, the third ZFN binding unit comprises at least 2, 3, 4, 5, 6, 7, 8, 9, or 10 zinc fingers and a third nuclease domain, wherein the zinc fingers are linked together, wherein the linked zinc fingers recognize a portion of a FX nucleotide sequence, and wherein the linked zinc fingers are further linked to the third nuclease domain. In some embodiments, the second ZFN unit further comprises a fourth ZFN binding unit. In some embodiments, the fourth ZFN binding unit comprises at least one zinc finger and a fourth nuclease domain. In some embodiments, the fourth ZFN binding unit comprises at least 2, 3, 4, 5, 6, 7, 8, 9, or 10 zinc fingers and a fourth nuclease domain, wherein the zinc fingers are linked together, and wherein the linked zinc fingers recognize a portion of a FX nucleotide sequence. In some embodiments, the fourth ZFN binding unit comprises at least 2, 3, 4, 5, 6, 7, 8, 9, or 10 zinc fingers and a fourth nuclease domain, wherein the zinc fingers are linked together, wherein the linked zinc fingers recognize a portion of a FX nucleotide sequence, and wherein the linked zinger fingers are further linked to the fourth nuclease domain. In some embodiments, the third ZFN binding unit and fourth ZFN binding unit bind to different sequences of the FX gene. In some embodiments, the third nuclease domain is a domain of an endonuclease. In some embodiments, the third nuclease domain is a domain of a restriction endonuclease. In some embodiments, the third nuclease domain is a domain of Fokl. In some embodiments, the fourth nuclease domain is a domain of an endonuclease. In some embodiments, the fourth nuclease domain is a domain of a restriction endonuclease. In some embodiments, the fourth nuclease domain is a domain of Fokl. In some embodiments, the third nuclease domain and fourth nuclease domain associate to comprise an active restriction endonuclease. In some embodiments, the third nuclease domain and fourth nuclease domain associate to comprise an active Fokl enzyme.

In some embodiments, provided herein is a method of producing a host cell, wherein the host cell has substantially no FX activity or partial FX activity, comprising inactivating the FX gene using a ZFN system, wherein the ZFN system comprises a first ZFN unit and a second TALEN unit that bind to different, non-overlapping portions of a FX gene sequence.

In some embodiments, any method of producing a host cell as provided herein, wherein the host cell has substantially no FX activity or partial FX activity, comprises inactivating the FX gene using a ZFN system, wherein the ZFN system comprises a coding vector encoding a first ZFN unit. In some embodiments, provided herein is a method of producing a host cell, wherein the host cell has substantially no FX activity or partial FX activity, comprises inactivating the FX gene using a ZFN system, wherein the ZFN system comprises a coding vector encoding a first ZFN unit, wherein the ZFN system comprises a coding vector encoding a second ZFN unit. In some embodiments, provided herein is a method of producing a host cell, wherein the host cell has substantially no FX activity or partial FX activity, comprising inactivating the FX gene using a ZFN system, wherein the ZFN system comprises a coding vector encoding a first ZFN unit, wherein the ZFN system comprises a coding vector encoding a first ZFN unit and a second ZFN unit.

In some embodiments, any method of producing a host cell provided herein, wherein the host cell has substantially no FX activity or partial FX activity, comprises inactivating the FX gene using a ZFN system, wherein the ZFN system comprises a first ZFN unit. In some embodiments, the method of producing a host cell, wherein the host cell has substantially no FX activity, comprises inactivating the FX gene using a ZFN system, wherein the ZFN system comprises a second ZFN unit. In some embodiments, the method of producing a host cell, wherein the host cell has substantially no FX activity or partial FX activity, comprises inactivating the FX gene using a ZFN system, wherein the ZFN system comprises a first ZFN unit and a second ZFN unit.

In some embodiments, the first ZFN binding unit comprises a group of linked zinc fingers, wherein the group of zinc fingers recognize a nucleotide sequence. In some embodiments, the nucleotide sequence is a sequence comprising a portion of a FX gene. In some embodiments, the nucleotide sequence is a sequence comprising a portion of a FX gene promoter. In some embodiments, the nucleotide sequence is a sequence comprising a portion of a sequence flanking a FX gene. In some embodiments, the sequence is at least about 80%, at least about 85%, at least about 90%, at least about 95%, or 100% homologous to a portion of a FX gene. In some embodiments, the sequence is at least about 80%, at least about 85%, at least about 90%, at least about 95%, or 100% homologous to a portion of a FX gene promoter. In some embodiments, the sequence is at least about 80%, at least about 85%, at least about 90%, at least about 95%, or 100% homologous to a portion of a sequence flanking a FX gene.

In some embodiments, provided herein is a method of producing a host cell comprising a partially or fully inactivated FX gene, comprising delivering a ZFN system to the host cell, wherein the ZFN system at least partially inactivates one or both FX genes in said host cell. In some embodiments, provided herein is a method of producing a host cell comprising a partially or fully inactivated FX gene, comprising delivering a vector comprising a ZFN system using a delivery vector, wherein the ZFN system at least partially inactivates one or both FX genes in said host cell. In some embodiments, the delivery vector is a virus vector. In some embodiments, the delivery vector is a lentivirus. In some embodiments, the delivery vector is an adenovirus.

In some embodiments a host cell wherein the FX gene has been inactivated has no functional FX activity. Such host cells may be referred to as a full FX knockout host cells. In another embodiment a host cell wherein the FX gene has been inactivated, e.g., partly inactivated, has partial FX activity. Such host cells may be referred to as a partial FX knockout host cells.

### Meganuclease

Generally, the meganuclease system used herein can comprise one or more meganucleases that allow for recognition of a DNA sequence and subsequent double-stranded DNA cleavage.

In some embodiments, provided herein is a method of producing a host cell, wherein the host cell has a substantially no FX activity or partial FX activity, comprising fully or partially inactivating the FX gene of the host cell using a meganuclease system. In some embodiments, the meganuclease has a DNA recognition sequence that is about 8 to about 35 nucleotide base pairs in length. In some embodiments, the meganuclease has a DNA recognition sequence that is about 12 to about 30 nucleotide base pairs in length. In some embodiments, the DNA recognition sequence is a sequence comprising a portion of a FX gene. In some embodiments, the DNA recognition sequence is a sequence comprising a portion of a FX gene promoter. In some embodiments, the DNA recognition sequence is a sequence comprising a portion of a nucleotide sequence flanking a host cell FX gene.

In some embodiments, provided herein is a method of producing a host cell comprising delivering a meganuclease system to the host cell. In some embodiments, provided herein is a method of producing a host cell comprising delivering a vector comprising a meganuclease system using a delivery vector. In some embodiments, the delivery vector is a virus vector. In some embodiments, the delivery vector is a lentivirus. In some embodiments, the delivery vector is an adenovirus.

### RNAi (RNA Inhibition)

In some embodiments, the FX gene is inactivated by RNA inhibition (RNAi).

Suitable examples of RNAi systems, include small interfering RNA (siRNA), antisense oligonucleotides (ASOs), microRNAs (miRNA), and a small hairpin RNA (shRNA).

In some embodiments, the FX gene is inactivated by a small interfering RNA (siRNA) system or antisense oligonucleotide (ASO) system, wherein a host cell will comprise the siRNA system or ASO system, respectively.

In some embodiments, the system comprises a siRNA or ASO nucleotide sequence that is about 10 to 200 nucleotides in length, or about 10 to 100 nucleotides in length, or about 15 to 100 nucleotides in length, or about 10 to 60 nucleotides in length, or about 15 to 60 nucleotides in length, or about 10 to 50 nucleotides in length, or about 15 to 50 nucleotides in length, or about 10 to 30 nucleotides in length, or about 15 to 30 nucleotides in length. In some embodiments, the siRNA or ASO nucleotide sequence is approximately 10-25 nucleotides in length. In some embodiments, the siRNA or ASO nucleotide sequence is approximately 15-25 nucleotides in length. In some embodiments, the siRNA or ASO nucleotide sequence is at least about 10, at least about 15, at least about 20, or at least about 25 nucleotides in length. In some embodiments, the siRNA system comprises a nucleotide sequence that is at least about 80%, at least about 85%, at least about 90%, at least about 95%, or 100% complementary to a region of a FX mRNA molecule. In some embodiments, the system comprises a nucleotide sequence that is at least at least about 80%, at least about 85%, at least about 90%, at least about 95%, or 100% complementary to a region of a FX pro-mRNA molecule. In some, embodiments, the system comprises a double stranded RNA molecule. In some embodiments, the system comprises a single stranded RNA molecule. In some embodiments, the host cell comprises a system as described in the any of the embodiments herein. In some embodiments, the host cell comprises a pro-siRNA nucleotide sequence that is processed into an active siRNA molecule as described in the any of the embodiments herein. In some embodiments, the host cell comprises a siRNA or ASO nucleotide sequence that is at least about 80%, at least about 85%, at least about 90%, at least about 95%, or 100% complementary to a region of a FX mRNA molecule. In some embodiments, the host cell comprises an expression vector encoding a siRNA molecule as described in any of the embodiments herein. In some embodiments, the host cell comprises an expression vector encoding a pro-siRNA molecule as described in the any of the embodiments herein.

In some embodiments, the FX gene is inactivated by a small interfering RNA (siRNA) system. Methods for identifying siRNA sequences suitable for FX gene inactivation are well known in the art. For example, general consideration for developing and identifying siRNA to target the FX gene include: a) first search sequences that are preferably 21-23 nucleotides in length (followed by reduction of sequence length as necessary), b) avoid regions within 50-100 base pairs of the start codon and the termination codon, c) avoid intron regions, d) avoid stretches of four or more bases, e.g., AAAA, e) avoid regions with GC content that is less than 30% or greater than 60%, f) avoid repeats and low sequence complexity, g) avoid single nucleotide polymorphism sites, and h) avoid sequences that are complementary to sequences in other off-target genes. See, e.g., Rules of siRNA design for RNA interference, Protocol Online, May 29, 2004; and Reynolds et al., Nat Biotechnol, 22, 2004.

In some embodiments, the FX gene in the host cell is inactivated by an antisense oligonucleotide (ASO) system. Methods for identifying ASO sequences suitable for FX gene inactivation are well known in the art.

In some embodiments, the system comprises a delivery vector. In some embodiments, the host cell comprises a delivery vector. In some embodiments, the delivery vector comprises the pro-siRNA and/or siRNA molecule.

In some embodiments, the FX gene is inactivated by a small hairpin RNA (shRNA; also referred to as a short hairpin RNA) system, wherein a host cell comprises the shRNA system. Gene inactivation by shRNA systems are well known in the art. In some embodiments, the shRNA system comprises a nucleotide sequence that is about 10 to 200 nucleotides in length, or about 10 to 100 nucleotides in length, or about 15 to 100 nucleotides in length, or about 10 to 60 nucleotides in length, or about 15 to 60 nucleotides in length, or about 10 to 50 nucleotides in length, or about 15 to 50 nucleotides in length, or about 10 to 30 nucleotides in length, or about 15 to 30 nucleotides in length. In some embodiments, the shRNA nucleotide sequence is approximately 10-25 nucleotides in length. In some embodiments, the shRNA nucleotide sequence is approximately 15-25 nucleotides in length. In some embodiments, the shRNA nucleotide sequence is at least about 10, at least about 15, at least about 20, or at least about 25 nucleotides in length. In some embodiments, the shRNA system comprises a nucleotide sequence that is at least about 80%, at least about 85%, at least about 90%, at least about 95%, or 100% complementary to a region of a FX mRNA molecule. In some embodiments, the shRNA system comprises a nucleotide sequence that is at least about 80%, at least about 85%, at least about 90%, at least about 95%, or 100% complementary to a region of a FX pro-mRNA molecule. In some embodiments, the shRNA system comprises a double stranded RNA molecule. In some embodiments, the shRNA system comprises a single stranded RNA molecule. In some embodiments, the host cell comprises a shRNA system as described in any of the embodiments herein. In some embodiments, the host cell comprises a pro-shRNA nucleotide sequence that is processed in an active shRNA nucleotide sequence as described in any of the embodiments herein. In some embodiments, the pro-shRNA molecule is composed of DNA. In some embodiments, the pro-shRNA molecule is a DNA construct. In some embodiments, the host cell comprises a shRNA nucleotide sequence that is at least about 80%, at least about 85%, at least about 90%, at least about 95%, or 100% complementary to a region of a FX mRNA molecule. In some embodiments, the host cell comprises an expression vector encoding a shRNA molecule as described in any of the embodiments herein. In some embodiments, the host cell comprises an expression vector encoding a pro-shRNA molecule as described in any of the embodiments herein.

In some embodiments, the shRNA system comprises a delivery vector. In some embodiments, the host comprises a delivery vector. In some embodiments, the delivery vector comprises the pro-shRNA and/or shRNA molecule.

### Types of Inactivation

In some embodiments, the FX gene is inactivated, wherein a host cell comprises a gene deletion. As used herein, “gene deletion” refers to removal of at least a portion of a DNA sequence, such as a single nucleic acid, from, or in proximity to, a gene, e.g., an FX gene. In some embodiments, the sequence subjected to gene deletion comprises an exonic sequence of a gene. In some embodiments, the sequence subjected to gene deletion comprises a promoter sequence of a gene, e.g., an FX gene. In some embodiments, the sequence subjected to gene deletion comprises a flanking sequence of a gene, e.g., an FX gene. In some embodiments, a portion of a gene sequence is removed from a gene, e.g., an FX gene. In some embodiments, a portion of the FX gene sequence is removed from, or in proximity to, the FX gene. In some embodiments, the complete gene sequence, e.g., FX gene sequence, is removed from a chromosome. In some embodiments, the complete FX gene sequence is removed from a chromosome. In some embodiments, the host cell comprises a gene deletion as described in any of the embodiments herein. In some embodiments, the host cell comprises a gene deletion in the FX gene. In some embodiments, the host cell comprises a gene deletion or chromosomal deletion in proximity to the FX gene.

In some embodiments, the FX gene in the host cell is inactivated, wherein the host cell comprises a gene addition or substitution. As used herein, “gene addition” or “gene substitution” refers to an alteration of a gene sequence, including insertion or substitution of one or more nucleotides or nucleotide base pairs. In some embodiments, the intronic sequence of the gene is altered. In some embodiments, the exonic sequence of the gene is altered. In some embodiments, the promoter sequence of the gene is altered. In some embodiments, the flanking sequence of the gene is altered. In some embodiments, one nucleotide or nucleotide base pair is added to a gene sequence. In some embodiments, at least one nucleotide or nucleotide base pair, or two consecutive nucleotides or nucleotide base pairs, are added to a gene sequence. In some embodiments, the host cell comprises a gene addition or substitution as described in any of the embodiments herein. In some embodiments, the host cell comprises a gene addition or gene substitution in the FX gene. In some embodiments, the host cell comprises a gene addition or gene substitution in the FX gene.

In some embodiments, the FX gene in the host cell is inactivated by a gene deletion, wherein deletion of at least one nucleotide or nucleotide base pair in a gene sequence results in a non-functional gene product. In some embodiments, the FX gene is inactivated by a gene deletion, wherein deletion of at least one nucleotide or nucleotide base pair of a gene sequence results in a gene product that no longer has the original gene product function or activity. In some embodiments, the FX gene is inactivated by a gene deletion, wherein deletion of at least one nucleotide or nucleotide base pair of a gene sequence results in a dysfunctional gene product.

In some embodiments, the FX gene is inactivated by a gene addition or substitution, wherein addition or substitution of at least one nucleotide or nucleotide base pair into the FX gene sequence results in a semi-functional or non-functional gene product. In some embodiments, the FX gene is inactivated by a gene inactivation, wherein incorporation or substitution of at least one nucleotide or nucleotide base pair to the FX gene sequence results in a gene product that no longer has the original gene product function or activity. In some embodiments, the FX gene is inactivated by a gene addition or substitution, wherein incorporation or substitution of at least one nucleotide or nucleotide base pair into the FX gene sequence results in a dysfunctional gene product. In some embodiments, the FX gene is inactivated by a small interfering RNA (siRNA) system or antisense oligonucleotide (ASO) system.

In some embodiments, the host cell comprises a non-functional FX gene product. In some embodiments, the host cell comprises a FX gene product that does not have the original FX gene product function or activity, respectively. In some embodiments, the host cell comprises a dysfunctional FX gene product.

In some embodiments, the host cell comprises an inactivated FX gene, wherein the inactivated FX gene will not express a full length, and functional, FX gene product (e.g., a full length FX polypeptide sequence), respectively. In some embodiments, the host cell comprises an inactivated FX gene, wherein the inactivated FX gene does not express an endogenous FX gene product, or expresses an endogenous FX gene product sequence at a reduced level as compared to a wild-type cell. In some embodiments, the host cell comprises an inactivated FX gene, wherein the inactivated FX gene will express a variant FX gene product, respectively. In some embodiments, the host cell comprises a variant FX gene product.

In some embodiments, the host cell comprises a partially inactivated FX gene, wherein the partially inactivated FX gene does not express a full length, and functional, FX gene product (e.g., a full length FX polypeptide sequence), respectively. In some embodiments, the host cell comprises a partially inactivated FX gene, wherein the partially inactivated FX gene does not express an endogenous FX gene product sequence, respectively. In some embodiments, the host cell comprises a partially inactivated FX gene, wherein the partially inactivated FX gene expresses a variant FX gene product, respectively. In some embodiments, the host cell comprises a variant FX gene product.

In some embodiments, the host cell comprises a delivery vector. In some embodiments, the delivery vector is a virus vector. In some embodiments, the delivery vector is a retrovirus or a retroviral vector. In some embodiments, the delivery vector is a lentivirus or lentiviral vector. In some embodiments, the delivery vector is an adenovirus or adenoviral vector.

In some embodiments, the host cell is a stable knockdown host cell. In some embodiments, the host cell is a stable FX knockdown cell line. In some embodiments, the host cell is a transient knockdown cell line. In some embodiments, the host cell is a transient FX knockdown cell line.

In some embodiments, the host cell further comprises an inactivated gene other than FX.

In general, host cells are transformed or transfected with a recombinant expression vector that comprises DNA encoding a desired protein. Additionally, the host cells of the present application can be a blank host cell. As used herein, “blank host” refers to a cell that does not contain an expression vector encoding a protein. In some embodiments, the blank host cell is a CHO cell. In some embodiments, the blank host cell is a mouse cell.

The host cell may have been engineered to have partial FX activity or substantially no FX activity, as described herein.

Also provided by the present application are host cells comprising nucleic acid molecules encoding a protein, e.g., a protein of interest, as described herein. Nucleic acid molecules provided herein include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acid molecules provided herein are preferably derived from human sources.

In certain embodiments, the nucleic acid molecules are substantially free from contaminating endogenous material. The nucleic acid molecules have preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, NY (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

In some embodiments, the host cell is capable of expressing, or expresses, a protein, e.g., a protein of interest, as described herein. In some embodiments, the host cell is capable of expressing a Fc-containing protein. In some embodiments, the host cell comprises a protein, e.g., an expressed Fc-containing protein. In some embodiments, the host cell comprises a heterologous protein. In some embodiments, the host cell comprises a Fc-containing protein. In some embodiments, the host cell is capable of secreting a protein. In some embodiments, the host cell is capable of secreting a Fc-containing protein.

In some embodiments, the host cell comprising FX gene inactivation, e.g. partial inactivation, is capable of expressing a protein at a similar output rate of the host cell prior to FX gene inactivation. In some embodiments, the host cell is capable of expressing a protein at the same output rate of the host cell prior to FX gene inactivation. In some embodiments, the host cell is capable of expressing a protein at about 70%, 75%, 80%, 85%, 90%, 95%, or 100% of the output rate of the host cell prior to FX gene inactivation.

In some embodiments, the host cell further comprises a gene modification. Optimization of a host cell for the purposes of producing a protein via gene modification is well known in the art and includes considerations pertaining to, for example, vector selection properties for integration methods and any other cellular property that would be desirable to manipulate for the production of a protein. In some embodiments, the gene modification is a targeted gene modification. In some embodiments, the gene modification is a knockout gene modification. In some embodiments, the gene modification is a knock-in gene modification.

Also provided are methods of evaluating a host cell for suitability of expression of proteins comprising determining FX activity, wherein a reduced level of FX activity is indicative of suitability.

### Determining a Level of FX Activity

In some embodiments, provided herein is a method of producing a host cell, wherein the host cell has partial FX activity, further comprising determining a level of FX activity. In some embodiments, provided herein is a method of producing a host cell comprising determining the level of FX activity, wherein a FX gene deletion or modification is detected. In some embodiments, provided herein is a method of producing a host cell comprising determining the level of FX activity, wherein a FX gene insertion or substitution is also detected. In some embodiments, provided herein is a method of producing a host cell comprising determining the level of FX activity, wherein a level of FX expression is determined prior to inactivating the FX gene in a host cell. In some embodiments, provided herein is a method of producing a host cell comprising determining the level of FX activity, wherein a level of FX activity is determined after using a CRISPR system to inactive the FX gene in a host cell. In some embodiments, provided herein is a method of producing a host cell comprising determining the level of FX, wherein a level of FX activity is determined after using a TALEN system to inactive the FX gene in a host cell. In some embodiments, provided herein is a method of producing a host cell comprising determining the level of FX activity, wherein a level of FX activity is determined after using a ZFN system to inactive the FX gene in a host cell. In some embodiments, provided herein is a method of producing a host cell comprising determining the level of FX gene inactivation, wherein a level of FX activity is determined after using a meganuclease system to inactivate the FX gene in a host cell. In some embodiments, provided herein is a method of producing a host cell comprising determining the level of FX gene inactivation, wherein a level of FX activity is determined after the FX gene is inactivated by a small interfering RNA (siRNA) system or antisense oligonucleotide (ASO) system.

In some of the methods disclosed herein, the FX activity level is determined at the DNA level. In some embodiments, the FX activity level is determined at the RNA level. In some embodiments, provided herein is a method of producing a host cell comprising determining the FX activity level using PCR. In some embodiments, provided herein is a method of producing a host cell comprising determining the FX activity level using PCR, wherein a variant sequence is detected. In some embodiments, provided herein is a method of producing a host cell comprising determining the FX activity level using qPCR. In some embodiments, provided herein is a method of producing a host cell comprising determining the FX activity level using qPCR.

In some of the methods e.g. of producing a host cell, disclosed herein, the FX activity level can be determined at the protein level. In some embodiments, any of the methods of producing a host cell provided herein comprises determining the host cell's FX activity level using immunohistochemistry. In some embodiments, the host cell's FX activity level is determined using a Western blot. In some embodiments, the host cell's FX activity level using flow cytometry.

In some embodiments, the level of FX gene inactivation is determined by comparing the level of FX activity after FX gene inactivation to a control value, e.g., FX activity in an unmodified cell. In some embodiments, the level of FX gene inactivation is determined by comparing a level of FX activity after FX gene inactivation to a level of FX activity prior to FX gene inactivation.

### Host Cells

The present application provides, in some aspects, a host cell for expression of a protein, wherein the host cell comprises substantially no FX activity or partial FX activity, as described herein and appropriate for particular aspects of the invention.

Any appropriate host cell can be used to produce the protein of interest, including a host cell derived from yeast, insect, amphibian, fish, reptile, bird, mammal, or human, or a hybridoma cell. The host cell can be an unmodified cell or cell line, or a cell line that has been genetically modified (e.g., to facilitate production of a biological product). In some embodiments, the host cell is a cell line that has been modified to allow for growth under desired conditions, such as in serum-free media, in cell suspension culture, or in adherent cell culture.

A mammalian host cell can be advantageous to use for antibodies intended for administration to humans. In some embodiments, the host cell is a Chinese hamster ovary (CHO) cell, which is a cell line used for the expression of many recombinant proteins. Additional mammalian cell lines commonly used for the expression of recombinant proteins include 293HEK cells, HeLa cells, COS cells, NIH/3T3 cells, Jurkat cells, NSO cells, and HUVEC cells. In other embodiments, the host cell is a recombinant cell which expresses an antibody.

Among the host cells that may be employed are eukaryotic cells, such as yeast or higher eukaryotic cells. Higher eukaryotic cells include insect cells and established cell lines of mammalian origin.

Examples of suitable mammalian host cell include the COS-7 line of monkey kidney cells (ATCC CRL 1651) (Gluzman et al, Cell, 23, 1981), L cells, 293 cells, C127 cells, 3T3 cells (ATCC CCL 163), Chinese hamster ovary (CHO) cells, or their derivatives such as Veggie CHO and related cell lines which grow in serum-free media (Rasmussen et al., Cytotechnology, 28, 1998), HeLa cells, BHK (ATCC CRL10) cell lines, and the CVI/EBNA cell line derived from the African green monkey kidney cell line CVI (ATCC CCL 70) as described by McMahan et al., EMBO J, 10, 1991, human embryonic kidney cells such as 293, 293 EBNA, or MSR 293, human epidermal A431 cells, human Colo205 cells, other transformed primate cell lines, normal diploid cells, cell strains derived from in vitro culture of primary tissue, primary explants, HL-60, U937, HaK, or Jurkat cells. Optionally, for example, mammalian cell lines such as HepG2/3B, KB, NIH 3T3 or S49, can be used as host cells.

In some embodiments, the host cell is a CHO cell. CHO cells are well known in the art. See, e.g., Xu et al., Nat Biotechnol, 29, 2011. In some embodiments, the host cell is a DP12 host cell. In some embodiments, the host cell is a DUXB-11 derived DHFR-deficient DP12 host cell. In some embodiments, the host cell is a CHO-K1 host cell. In some embodiments, the host cell is a DHFR-positive CHO-K1 host cell. In some embodiments, the host cell is a CHOK1M host cell.

In particular embodiments, the integration of the polypeptide or protein or fragment thereof is targeted to a particular site in the CHO cell genome (targeted integration) as described in WO 2019/126634, whose disclosure referring to the targeted integration host and methods of making thereof, is hereby incorporated by reference. In other embodiments, the integration of the polypeptide or protein or fragment thereof into the CHO cell genome is random (random integration).

In some embodiments, the host cell is a mouse host cell. In some embodiments, the host cell is a Sp2/0 host cell. In some embodiments, the host cell is a NSO host cell.

In some embodiments, the host cell is a hybridoma. In some embodiments, the hybridoma is an antibody-producing cell, wherein the antibody-producing cell is collected from a host following immunization of the host with an antigen. In some embodiments, the antibody-producing cell is fused with a myeloma cell. In some embodiments, the host cell is a mouse myeloma-derived cell line.

Alternatively, the host cell can be a lower eukaryote such as yeast. Suitable yeasts include Saccharomyces cerevisiae, Schizosaccharomyces pombe, Kluyveromyces strains, Candida, or any yeast strain capable of expressing heterologous polypeptides.

### Cells Engineered to Produce a Protein

The present application, in some aspects, provides a host cell engineered to produce a protein as described in the embodiments herein. The present application, in other aspects, provides methods for making a host cell as described in the embodiments herein. In some aspects, cell lines were developed in a targeted integration host as described in WO 2019/126634, the disclosure of which refers to a targeted integration host (TI host cell) and methods of making thereof, is hereby incorporated by reference.

In some embodiments, the host cell is engineered to produce a protein.

In some embodiments, provided herein is a method for making a host cell engineered to produce a protein comprising: a) transforming the host cell with an expression vector comprising a nucleic acid encoding the protein.

Methods for transforming a host cell using an expression vector are well known in the art. See, for example, Kim et al., Anal. Bioanal. Chem, 397, 2010. Method for transforming a host cell including, but are not limited to, transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan.

Expression systems and constructs in the form of plasmids, expression vectors, transcription or expression cassettes which comprise at least one protein described herein are also provided.

In certain embodiments, a plasmid, expression vector, transcription or expression cassette provided herein comprises a polynucleotide encoding at least one protein.

In some embodiments, expression vectors used in the host cells contain sequences for plasmid maintenance and for cloning and expression of exogenous nucleotide sequences. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the protein to be expressed, and a selectable marker element. Each of these sequences is discussed below.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, CA), or other methods known to the skilled artisan. The selection of suitable enzymes to accomplish this purpose will be readily apparent to one of ordinary skill in the art.

An origin of replication is typically a part expression vector purchased commercially, and the origin aids in the amplification of the vector in a host cell. If the vector of choice does not contain an origin of replication site, one may be chemically synthesized based on a known sequence, and ligated into the vector. For example, various viral origins of replication (e.g., SV40, polyoma, adenovirus, vesicular stomatitis virus (VSV), or papillomaviruses such as human papillomavirus (HPV) or bovine papillomavirus (BPV) origins of replication) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antibody light or heavy chain. As a result, increased quantities of a polypeptide are synthesized from the amplified DNA.

A ribosome-binding site is usually necessary for translation initiation of mRNA and is characterized by, for example, a Kozak sequence. The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

Expression and cloning vectors of the invention will typically contain a promoter that is recognized by the host organism and operably linked to the molecule encoding the polypeptide. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe gene to which they are operably linked, that is, with little or no control over gene expression. A large number of promoters, recognized by a variety of potential host cells, are well known. A suitable promoter is operably linked to the DNA encoding e.g., heavy chain or light chain, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thomsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene Prinster et al., 1982, Nature 296:39-42; or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feta-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al., 1985, Nature 315:338-340; Kollias et al., 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding light chain or heavy chain of the invention by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alphafeto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides that are functional in mammalian host cells include the following: the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-I receptor signal peptide described in U.S. Pat. No. 4,968,607; the type II interleukin-1 receptor signal peptide described in EP Patent No. 0 460 846.

The vector may contain one or more elements that facilitate expression when the vector is integrated into the host cell genome. Examples include an EASE element (Aldrich et al. 2003 Biotechnol Prog. 19: 1433-38) and a matrix attachment region (MAR). MARs mediate structural organization of the chromatin and may insulate the integrated vector from “position” effect. Thus, MARs are particularly useful when the vector is used to create stable transfectants. A number of natural and synthetic MAR-containing nucleic acids are known in the art, e.g., U.S. Pat. Nos. 6,239,328; 7,326,567; 6,177,612; 6,388,066; 6,245,974; 7,259,010; 6,037,525; 7,422,874; 7,129,062.

Expression vectors provided by the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding a protein sequence has been inserted into the proper site of the vector, the completed vector may be inserted into a suitable host cell for amplification and/or polypeptide expression.

Methods for making a vector comprising a nucleic acid encoding a protein, such as an Fc-containing protein, are well known in the art. See, e.g., U.S. Pat. No. 7,923,221.

Construction of suitable vectors comprising a protein and the desired coding and control sequences employ standard ligation techniques. Isolated plasmids or DNA fragments are cleaved, tailored, and re-ligated in the form desired to form the plasmids required. The methods employed are not dependent on the DNA source, or intended host.

In some embodiments, there is provided a method of making a protein further comprising determining an optimal ratio of the polynucleotide for introduction into a host cell. In some embodiments, mass spectrometry is used to determine protein yield, and the ratio is adjusted to maximize protein yield. In some embodiments, dual antigen ELISA is used to determine protein yield, such as a Fc-containing protein, and the ratio is adjusted to maximize protein yield.

### Protein of Interest

The present application, in some aspects, relates to methods for preparing a protein of interest. The present application, in some aspects, provides a protein comprising a glycan structure, e.g., a preparation of a protein comprising a glycan structure, wherein the protein is in a predetermined ratio of fucosylated and afucosylated forms of the protein, produced by any of the methods disclosed herein.

In some embodiments, the protein is a Fc-containing protein. In some embodiments, the Fc-containing protein comprises a Fc domain. In some embodiments, the Fc-containing protein comprises one or more Fc domains. In some embodiments, the Fc-containing protein comprises two Fc domains.

In some embodiments, the Fc-containing protein comprises a heavy chain or a fragment thereof. In some embodiments, the Fc-containing protein comprises at least one heavy chain. In embodiments, the Fc-containing protein comprises one or more heavy chains. In some embodiments, the Fc-containing protein comprises two heavy chains.

In some embodiments, the Fc-containing protein is an antibody. In some embodiments, the Fc-containing protein is a full length antibody. In some embodiments, the antibody, e.g., full length antibody is a human antibody. In some embodiments, the antibody, e.g., full length antibody is a humanized antibody. In some embodiments, the antibody, e.g., full length antibody is a monoclonal antibody. In some embodiments, the antibody, e.g., full length antibody is a chimeric antibody. In some embodiments, the antibody, e.g., full length antibody is a bispecific antibody. In some embodiments, the antibody, e.g., the full length antibody is a multispecific antibody.

In some embodiments, the Fc-containing protein is a Fc-containing fusion protein. In some embodiments, the Fc-containing fusion protein comprises one or more Fc domains.

In some embodiments, the Fc domain of the Fc-containing fusion protein prolongs a plasma half-life of the Fc-containing fusion protein. In some embodiments, the Fc domain of the Fc-containing fusion protein prolongs the biological activity of the Fc-containing fusion protein. In some embodiments, the Fc domain of the Fc-containing fusion protein decreases the rate of renal clearance of the Fc-containing fusion protein. In some embodiments, the Fc domain of the Fc-containing fusion protein increases the solubility of the Fc-containing fusion protein. In some embodiments, the Fc domain of the Fc-containing fusion protein increases the stability of the Fc-containing fusion protein.

Fc-containing fusion proteins are well known in the art. See, e.g., Czajkowsky et al., EMBO Mol Med, 4, 2012, 1015-1028. In some embodiments, the Fc-containing fusion protein is an immunoadhesin. In some embodiments, the Fc-containing fusion protein is a cytokine-Fc fusion protein.

In some embodiments, the protein is a multimeric protein. In some embodiments, the Fc-containing protein is a multimeric protein. In some embodiments, the Fc-containing fusion protein is a multimeric protein.

In some embodiments, the protein is conjugated to an agent. In some embodiments, the protein is conjugated to at least about 2, 3, 4, 5, 6, 7, 8, 9, or 10 molecules of an agent. In some embodiments, the protein is conjugated to about 2-10, about 4-10, about 6-10, or about 8-10 molecules of an agent. In some embodiments, the Fc-containing protein is conjugated to an agent, wherein the agent is conjugated to the Fc domain of the Fc-containing protein. In some embodiments, the agent is a therapeutic agent. In some embodiments, the therapeutic agent is a small molecule therapeutic agent. In some embodiments, the therapeutic agent is a chemotherapeutic agent. In some embodiments, the agent is a detection agent. In some embodiments, the detection agent is a radiolabel. In some embodiments, the detection agent is a fluorescent label. In some embodiments, the detection agent is an immunolabel. In some embodiments, the protein is a companion diagnostic. In some embodiments, the Fc-containing protein is a companion diagnostic.

In some embodiments, the protein comprises a post-translational modification. In some embodiments, the post-translational modification is non-enzymatically produced. In some embodiments, the post-translational modification is enzymatically produced. In some embodiments, the post-translational modification is selected from the group consisting of a disulfide pairing, a deamidation, an oxidation, and a N-terminal glutamine cyclization.

In some embodiments, the protein of interest that can be produced using the methods described in this disclosure include, without limitation, therapeutic antibodies such as anti-HER receptor family antibodies (such as anti-HER1 (EGFR), anti-HER2, anti-HER3 and anti-HER4); anti-CD protein antibodies (such as anti-CD3, anti-CD4, anti-CD8, anti-CD19, anti-CD20, anti-CD21, anti-CD22, anti-CD25, anti-CD33, anti-CD34, anti-CD38, anti-CD52); anti-IL-8 antibodies; anti-VEGF antibodies; anti-CD40 antibodies, anti-CD11a antibodies; anti-CD18 antibodies; anti-IgE antibodies; anti-Apo-2 receptor antibodies; anti-Tissue Factor (TF) antibodies; anti-cell adhesion molecules such as LFA-1, Mol, p150,95, VLA-4, ICAM-1, VCAM, anti-human α₄β₇ integrin antibodies, anti-human αᵥβ₈ integrin antibodies, anti-αvβ3 antibodies including either α or ®β or subunits thereof (e.g. anti-CD11a, anti-CD18 or anti-CD11b antibodies); anti-EGFR antibodies; anti-Fc receptor antibodies; anti-carcinoembryonic antigen (CEA) antibodies; anti-human renal cell carcinoma antibodies; anti-human colorectal tumor antibodies; anti-human melanoma antibody R24 directed against GD3 ganglioside; anti-human squamous-cell carcinoma; antibodies directed against breast epithelial cells; antibodies that bind to colon carcinoma cells; anti-EpCAM antibodies; anti-GpIIb/IIIa antibodies; anti-RSV antibodies; anti-CMV antibodies; anti-HIV antibodies; anti-hepatitis antibodies; anti-CA 125 antibodies; anti-human 17-1A antibodies; and anti-human leukocyte antigen (HLA) antibodies, and anti-HLA DR antibodies; anti-growth factors such as vascular endothelial growth factor (anti-VEGF) or fragments; anti-IgE; anti-blood group antigens; anti-flk2/flt3 receptor; and anti-obesity (OB) receptor. Other exemplary proteins to which therapeutic antibodies are designed include anti-amyloid antibodies, anti-alpha-synuclein (e.g.: prasinezumab), anti-amyloid-beta, anti-growth hormone (GH), including human growth hormone (hGH) and bovine growth hormone (bGH); growth hormone releasing factor; parathyroid hormone; thyroid stimulating hormone; lipoproteins; [symbol] -1-antitrypsin; insulin A-chain; insulin B-chain; proinsulin; follicle stimulating hormone; calcitonin; luteinizing hormone; glucagon; clotting factors such as factor VIIIC, tissue factor or von Willebrands factor; anti-clotting factors such as Protein C; atrial natriuretic factor; lung surfactant; a plasminogen activator, such as urokinase or tissue-type plasminogen activator (t-PA); bombazine; thrombin; tumor necrosis factor-α and -β; enkephalinase; RANTES (regulated on activation normally T-cell expressed and secreted); human macrophage inflammatory protein (MIP-1- [symbol] ); serum albumin such as human serum albumin (HSA); mullerian-inhibiting substance; relaxin A-chain; relaxin B-chain; prorelaxin; mouse gonadotropin-associated peptide; DNase; inhibin; activin; receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-0; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins (IGFBPs); erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; and biologically active fragments or variants of any of the above-listed polypeptides. Many other antibodies and/or other proteins may be used in accordance with the instant invention, and the above lists are not meant to be limiting.

Therapeutic antibodies of particular interest may include those that are commercially available, in clinical practice or in development, such as AVASTIN® (bevacizumab), HERCEPTIN® (trastuzumab), LUCENTIS® (ranibizumab), RAPTIVA® (efalizumab), RITUXAN® (rituximab), ACTEMRA® (tocilizumab—anti-IL-6 receptor), XOLAIR® (omalizumab), OCREVUS® (ocrelizumab—anti-CD20 antibody), PERJETA® (pertuzumab—HER dimerization inhibitors (HDIs)), TECENTRIQ® (anti-PD-L1 antibody), LUNSUMIO® or COLUMVI™ (anti-CD20×anti-CD3 bispecific antibody), VABYSMO® (anti-VEGF-A×anti-angiopoietin-2 bispecific antibody), anti-CD79b antibody, anti-OX40 ligand, anti-oxidized LDL (oxLDL), anti-amyloid beta (Abeta), anti-CD4 (MTRX1011A), anti-EGFL7 (EGF-like-domain 7), anti-IL13, Apomab (anti-DR5-targeted pro-apoptotic receptor agonist (PARA), anti-BR3 (CD268, anti-BLyS receptor 3, anti-BAFF-R, (BAFF Receptor), anti-TIGIT (anti-T-cell immunoreceptor with immunoglobulin (Ig) and immunoreceptor tyrosine-based inhibitory motif domains) antibodies, astegolimab (anti-ST2, an IL-33 receptor), anti-beta 7 integrin subunit, anti-av3s integrin antibodies, dacetuzumab (Anti-CD40), GA101 (obinutuzumab—anti-CD20 monoclonal antibody), MetMAb (anti-MET receptor tyrosine kinase), cevostamab (anti-Fc receptor-homolog 5 (FcRH5)×anti-CD3 bispecific antibody), anti-neuropilin-1 (NRP1), rhuMAb IFN alpha, etc. Many other antibodies and/or other proteins may be used in accordance with the instant invention, and the above lists are not meant to be limiting.

In an embodiment, the therapeutic antibody is selected from AVASTIN® (bevacizumab), HERCEPTIN® (trastuzumab), LUCENTIS® (ranibizumab), RAPTIVA® (efalizumab), RITUXAN® (rituximab), ACTEMRA® (tocilizumab—anti-IL-6 receptor), XOLAIR® (omalizumab), OCREVUS® (ocrelizumab—anti-CD20 antibody), PERJETA® (pertuzumab—HER dimerization inhibitors (HDIs)), TECENTRIQ® (anti-PD-L1 antibody), LUNSUMIO® or COLUMVI™M (anti-CD20×anti-CD3 bispecific antibody), VABYSMO® (anti-VEGF-A×anti-angiopoietin-2 bispecific antibody), anti-CD79b antibody, anti-OX40 ligand, anti-oxidized LDL (oxLDL), anti-amyloid beta (e.g., trontinemab), anti-CD4 (MTRX1011A), anti-EGFL7 (EGF-like-domain 7), anti-IL13, Apomab (anti-DR5-targeted pro-apoptotic receptor agonist (PARA), anti-BR3 (CD268, anti-BLyS receptor 3, anti-BAFF-R, (BAFF Receptor), anti-TIGIT (anti-T-cell immunoreceptor with immunoglobulin (Ig) and immunoreceptor tyrosine-based inhibitory motif domains) antibodies, astegolimab (anti-ST2, an IL-33 receptor), anti-beta 7 integrin subunit, anti-αvβ8 integrin antibodies, dacetuzumab (Anti-CD40), GA101 (obinutuzumab—anti-CD20 monoclonal antibody), MetMAb (anti-MET receptor tyrosine kinase), cevostamab (anti-Fc receptor-homolog 5 (FcRH5)×anti-CD3 bispecific antibody), anti-neuropilin-1 (NRP1), and rhuMAb IFN alpha.

### Culture Medium and Methods of Culturing

The host cells used to produce a desired protein described herein may be cultured in a variety of culture media. Generally, the fucose content of said media will be known. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth Enz, 58, 1979, Barnes et al., Anal Biochem, 102, 1980, U.S. Pat. Nos. 4,767,704; 4,657,866; 4,927,762; 4,560,655; or 5,122,469; International Patent Application Nos. WO 90/03430 or WO 87/00195; or U.S. Pat. Reissue No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. In some embodiments, the culture medium further comprises a glucose source. In some embodiments, the culture medium further comprises a mannose source. Any other necessary supplements may also be included at appropriate concentrations that would be known to those skilled in the art.

In some embodiments, the culture medium is free of a fucose source. In some embodiments, the culture medium comprises a fucose source. As used herein, “fucose source” refers to a moiety comprising fucose involved in the fucosylation pathway. In some embodiments, the fucose source is a fucose. In some embodiments, the fucose is L-fucose. In some embodiments, the fucose is L-fucose-1-phosphate. In some embodiments, the fucose source is GDP-fucose. In some embodiments, the culture medium comprises a fucose source, wherein the amount of the fucose source in the culture medium is between about 0.01 mM and about 1 mM. In some embodiments, the fucose source is between about 0.01 mM and about 1 mM. In some embodiments, the fucose source is between about 0.01 mM and about 0.1 mM, about 0.01 mM and about 0.09 mM, about 0.01 mM and about 0.08 mM, about 0.01 mM and about 0.07 mM, about 0.01 mM and about 0.06 mM, about 0.01 mM and about 0.05 mM, about 0.01 mM and about 0.04 mM, about 0.01 mM and about 0.03 mM, about 0.01 mM and about 0.02 mM, about 0.02 mM and about 0.1 mM, about 0.02 mM and about 0.09 mM, about 0.02 mM and about 0.08 mM, about 0.02 mM and about 0.08 mM, about 0.02 mM and about 0.07 mM, about 0.02 mM and about 0.06 mM, about 0.02 mM and about 0.05 mM, about 0.02 mM and about 0.04 mM, about 0.02 mM and about 0.03 mM, about 0.03 mM and about 0.1 mM, about 0.03 mM and about 0.09 mM, about 0.03 mM and about 0.08 mM, about 0.03 mM and about 0.07 mM, about 0.03 mM and about 0.06 mM, about 0.03 mM and about 0.05 mM, about 0.03 mM and about 0.04 mM, about 0.04 mM and about 0.1 mM, about 0.04 mM and about 0.09 mM, about 0.04 mM and about 0.08 mM, about 0.04 mM and about 0.07 mM, about 0.04 mM and about 0.6 mM, about 0.04 mM and about 0.05 mM, about 0.05 mM and about 0.1 mM, about 0.05 mM and about 0.09 mM, about 0.05 mM and about 0.08 mM, about 0.05 mM and about 0.07 mM, about 0.05 mM and about 0.06 mM, about 0.06 mM and about 0.1 mM, about 0.06 mM and about 0.09 mM, about 0.06 mM and about 0.08 mM, about 0.06 mM and about 0.07 mM, about 0.07 mM and about 0.1 mM, about 0.07 mM and about 0.09 mM, about 0.07 mM and about 0.08 mM, about 0.08 mM and about 0.1 mM, about 0.08 mM and about 0.9 mM, or about 0.09 mM and about 0.1 mM. In some embodiments, the fucose source is about 0.01 mM, about 0.02 mM, about 0.03 mM, about 0.04 mM, about 0.05 mM, about 0.06 mM, about 0.07 mM, about 0.08 mM, about 0.09 mM, about 0.1 mM, about 0.11 mM, about 0.12 mM, about 0.13 mM, about 0.14 mM, about 0.15 mM, about 0.16 mM, about 0.17 mM, about 0.18 mM, about 0.19 mM, about 0.2 mM, about 0.3 mM, about 0.4 mM, about 0.5 mM, about 0.6 mM, about 0.7 mM, about 0.8 mM, about 0.9 mM, or about 1 mM.

In certain embodiments, when added, the amount of fucose added is at or above the saturation level of the antibody—the concentration where all the antibody molecules that are made by the cells are fully fucosylated. Such concentration could be anywhere above about 0.1-5 mM fucose (Louie, S.; Haley, B.; Marshall, B.; Heidersbach, A.; Yim, M.; Brozynski, M.; Tang, D.; Lam, C.; Petryniak, B.; Shaw, D.; Shim, J.; Miller, A.; Lowe, J. B.; Snedecor, B.; Misaghi, S., FX knockout CHO hosts can express desired ratios of fucosylated or afucosylated antibodies with high titers and comparable product quality. Biotechnol Bioeng 2017, 114, (3), 632-644).

Methods of culturing a host cell in a culture medium are well known to those in the art. See, e.g., Li et al., MAbs, 2, 2010. In some embodiments, culturing a host cell comprises culturing the host cell in a culture medium free of the fucose source prior to culturing the host cell in the culture medium comprising the fucose source. In some embodiments, the fucose source is present in the culture medium at the beginning of the culturing step. In some embodiments, the fucose source is added to the culture medium during the culturing step. In some embodiments, the fucose source is added at a cell density of, for example, at least 2×10⁵ cells/mL. In some embodiments, the fucose source is added at an oxygen level of, for example, 50% dissolved 0₂. In some embodiments, the fucose source is added at a glucose level of, for example, 6 g/L. In some embodiments, the fucose source is present in the culture medium at the beginning of the culturing step, and wherein a fucose source is added during the culturing step. In some embodiments, the fucose source is added to the culture medium during the culturing step via bolus addition. In some embodiments, the fucose source is added to the culture medium during the culturing step via continuous feeding. In some embodiments, the fucose source is added to the culture medium via bolus addition. In some embodiments, the fucose source is added to the culture medium via continuous feeding. In some embodiments, the fucose source is a fucose. In some embodiments, the fucose is L-fucose. In some embodiments, the fucose is L-fucose-1-phosphate. In some embodiments, the fucose source is GDP-fucose.

The culture conditions, such as temperature, pH, and the like, can be those previously used with the host cell selected for expression, and will be apparent to the ordinarily skilled artisan. In some embodiments, the culturing step is carried out at lower than about 370 C, 36° C., 35° C., 340 C, 33° C., 320 C, 31° C., or 30° C. In some embodiments, the culturing step is carried out at lower than about 37° C. In some embodiments, the culturing step is carried out at lower than about 34° C. In some embodiments, the culturing step is carried out at about 37° C. In some embodiments, the culturing step is carried out at about 34° C. In some embodiment, the culturing step is carried out at an initial temperature and then shifted to a second temperature. In some embodiments, the initial temperature is about 37° C. and the second temperature is about 34° C. In some embodiments, the initial temperature is about 34° C. and the second temperature is about 37° C.

Generally, the production of proteins is done on a large scale (such as a commercial/manufacture scale). To achieve a population of a host cell suitable for commercial scale production, one of ordinary skill in the art will recognize the utility using a stepwise approach to expanding a host cell population. For example, the process involves growing a desired host cell on a smaller scale to allow for an increase in the host cell population, such as a seed train. To further increase the population of the host cell, methods generally involved using the seed train to inoculate a larger culture tank, such as an inoculum tank. Often, a series of inoculum tanks of increasing size are used to expand the population of a host cell, such as an inoculum train. This process will provide a suitable population of a host cell for culture in a production culture. In some embodiments, the production culture is a 1000 L culture tank.

In some embodiments, there is provided a method of making a protein comprising culturing the host cell using a batch feed method. In some embodiments, there is provided a method of making a protein comprising culturing the host cell using a continuous feed method. In some embodiments, there is provided a method of making a Fc-containing protein comprising culturing the host cell using a feed method comprising a batch feed method and a continuous feed method.

The present application, in other aspects, provides a cell culture comprising any host cell described in the embodiments herein. In some embodiments, the cell culture can further comprise afucosylated and fucosylated forms of a protein, e.g., as part of the cell culture medium. In some embodiments, the cell culture can further comprise afucosylated and fucosylated forms of a protein at a specific ratio. In some embodiments, provided herein is a cell culture comprising a host cell engineered to express a protein, wherein the host cell comprises substantially no FX activity or partial FX activity, and a culture medium comprising a fucose source at about 0.01 mM to about 1 mM. In some embodiments, provided herein is a cell culture comprising a host cell engineered to express a protein, wherein the host cell comprises substantially no FX activity, or partial FX activity, and wherein the host cell is a knockout host cell (full knock out or partial knock out depending on the aspect of the invention employed) and a culture medium optionally comprising a fucose source at about 0.01 mM to about 1 mM. In some embodiments, provided herein is a cell culture comprising a host cell engineered to express a protein, wherein the host cell comprises substantially no FX activity or partial FX activity, and a culture medium optionally comprising a fucose source at about 0.01 mM to about 1 mM, wherein the protein is a Fc-containing protein. In some embodiments, provided herein is a cell culture comprising a host cell engineered to express a protein, wherein the host cell comprises substantially no FX activity or partial FX activity, and wherein the host cell is a full or partial FX gene knockout host cell, and a culture medium optionally comprising a fucose source at about 0.01 mM to about 1 mM, wherein the protein is a Fc-containing protein. In some embodiments, provided herein is a cell culture comprising a host cell engineered to express a protein, wherein the host cell comprises substantially no FX activity or partial FX activity, and a culture medium optionally comprising a fucose source at about 0.01 mM to about 1 mM, wherein the protein is an antibody. In some embodiments, provided herein is a cell culture comprising a host cell engineered to express a protein, wherein the host cell comprises substantially no FX activity or partial FX activity, and wherein the host cell is a knockout host cell, and a culture medium optionally comprising a fucose source at about 0.01 mM to about 1 mM, wherein the protein is an antibody. In some embodiments, provided herein is a cell culture comprising a host cell that expresses fucosylated and afucosylated forms of a protein at a predetermined ratio. In some embodiments, the host cell comprises substantially no FX activity. In some embodiments, the host cell comprises partial FX activity.

In some embodiments, the host cell is a mammalian cell. In some embodiments, the host cell is a CHO cell. In some embodiments, the fucose source is a fucose. In some embodiments, the fucose is L-fucose. In some embodiments, the fucose is L-fucose-1-phosphate. In some embodiments, the fucose source is GDP-fucose.

In some embodiments, the cell culture maintains a host cell in an environment for growth. In some embodiments, the cell culture maintains a host cell in an environment for the production of a protein. In some embodiments, the protein is produced in fucosylated and afucosylated forms at a predetermined ratio.

In some embodiments, the culture medium comprises a fucose source at about 0.01 mM to about 1 mM. In some embodiments, the culture medium comprises a fucose source at about 0.01 mM to about 0.1 mM. In some embodiments, the fucose source is a fucose. In some embodiments, the fucose is L-fucose. In some embodiments, the fucose is L-fucose-1-phosphate. In some embodiments, the fucose source is GDP-fucose.

In some embodiments, the cell culture is a seed culture. In some embodiments, the cell culture is an inoculum culture. In some embodiments, the inoculum culture is a primary inoculum culture. In some embodiments, the inoculum culture is a secondary inoculum. In some embodiments, the cell culture system comprises a production culture.

In some embodiments, the cell culture is maintained at a specified temperature. In some embodiments, the specified temperature is about 15° C. to about 45° C. In some embodiments, the specified temperature is about 30° C. In some embodiments, the specified temperature is less than about 37° C. In some embodiments, the specified temperature is less than about 35° C. In some embodiments, the specified temperature is less than about 34° C.

In some embodiments, the cell culture is maintained at a specified pH. In some embodiments, the cell culture is maintained at a specified dissolved oxygen concentration. In some embodiments, the cell culture is maintained at a specified nutrient level.

In some embodiments, the cell culture system comprises a protein as described in the embodiments herein. In some embodiments, the cell culture comprises a plurality of proteins as described in the embodiments herein. In some embodiments, the cell culture comprises a composition comprising a protein as described in the embodiments herein.

### Compositions

The present application, in some aspects, provides compositions comprising a protein made by any of the methods described herein. In some embodiments, the composition comprises afucosylated and fucosylated forms of the protein at a pre-determined ratio that provides the desired ADCC function.

In some embodiments, the fucosylated form of the protein comprises fucose at the reducing end of a glycan structure. In some embodiments, the fucosylated form of the protein comprises fucose at the reducing end of a glycan structure, wherein the fucose is covalently attached to a first N-acetylglucosamine (GlcNAc) moiety of the reducing end of the glycan structure. In some embodiments, the glycan structure comprises a fucose moiety at the reducing end of the glycan structure. In some embodiments, the fucose moiety is a single fucose molecule covalently bound to the glycan structure. In some embodiments, the glycan structure comprises an L-fucose. In some embodiments, the protein comprises two or more glycan structures.

In some embodiments, the composition comprising a plurality of proteins is a cell culture medium. In some embodiments, the cell culture medium is a nutrient medium. Nutrient media contains all elements needed for host cell growth. In some embodiments, the cell culture medium is a minimal medium. Minimal media contains the minimum nutrients possible for host cell growth, for example, generally without the presence of amino acids. In some embodiments, the cell culture medium is a selective medium. Selective media comprises an agent that inhibits growth of a select organism.

In some embodiments, the cell culture medium further comprises a cell culture medium nutrient for cell support and/or growth. In some embodiments, the cell culture medium nutrient is selected from, for example: proteins; peptides; amino acids; carbohydrates; metals and minerals, for example calcium, magnesium, iron; trace metals, for example, phosphates and sulphates; buffers; pH indicators, for example, phenol red, bromo-cresol purple; and antimicrobial agents.

In some embodiments, the cell culture medium further comprises a host cell. In some embodiments, the culture medium is substantially devoid of a host cell.

In some embodiments, the cell culture medium comprises a fucose source. In some embodiments, the fucose source is a fucose. In some embodiments, the fucose is L-fucose. In some embodiments, the fucose is L-fucose-1-phosphate. In some embodiments, the fucose source is GDP-fucose.

In some embodiments, the composition is a cell lysate. In some embodiments, the cell lysate comprises a plurality of proteins and host cell components. In some embodiments, the cell lysate is a centrifuged cell lysate. In some embodiments, the cell lysate comprises a precipitated portion of the cell lysate and a supernatant portion of the cell lysate. In some embodiments, the cell lysate comprises a pelleted portion of the cell lysate and a supernatant portion of the cell lysate.

In some embodiments, the composition is an eluate from a protein purification column. As used herein, “eluate” refers to any fluid that passes through a protein purification column. In some embodiments, the eluate comprises a fluid that is isolated from a flow-through fluid. In some embodiments, the eluate comprises a fluid that is isolated from a wash fluid. In some embodiments, the eluate comprises a fluid that is isolated from one or more wash fluids. In some embodiments, the eluate comprises a fluid that is isolated from an elution fluid.

In some embodiments, the composition is a library of proteins, wherein at least two of the proteins of the plurality of proteins are different. In some embodiments, the library comprises at least two proteins that bind to different antigens. In some embodiments, the library comprises at least two proteins that bind to different epitopes. In some embodiments, the different proteins are contained in different vessels. In some embodiments, the present invention provides libraries comprising at least 2, 3, 4, 5, 10, 30, 100, 250, 500, 750, 1000, 2500, 5000, 7500, 10000, 25000, 50000, 75000, 100000, 250000, 500000, 750000, 1000000, 2500000, 5000000, 7500000, 10000000, or more than 10000000 different proteins.

The present application provides large-scale batches (e.g., commercial batches or batches at manufacture scale) of any of the compositions described in the embodiments herein. For example, in some embodiments, the batch comprises fucosylated and afucosylated forms of a protein at a predetermined ratio. In some embodiments, the batch comprises fucosylated and afucosylated forms of an antibody at a predetermined ratio.

In some embodiments, the batch comprises at least about 5 g, 10 g, 50 g, 100 g, 200 g, 300 g, 400 g, 500 g, 600 g, 700 g, 800 g, 900 g, 1,000 g, 1,500 g, 2,000 g, 2,500 g, 3,000 g, 3,500 g, 4,000 g, 4,500 g, or 5,000 g of a protein, wherein the fucosylated and afucosylated forms of the protein are at a predetermined ratio. In some embodiments, the batch comprises at least about 5-5,000 g, 50-4,000 g, or about 100-1,000 g of a protein, wherein the fucosylated and afucosylated forms of the protein are at a predetermined ratio.

In some embodiments, the batch is in a form for drug storage. In some embodiments, the batch is in a form for product transportation. In some embodiments, the batch is in a form for administration to an individual in need thereof. In some embodiments, the batch is lyophilized. In some embodiments, the batch is not conjugated to an agent. In some embodiments, the batch is conjugated to an agent. In some embodiments, the batch further comprises a formulation component.

In some embodiments, the batch is a cell culture medium. In some embodiments the batch is a cell lysate.

In some embodiments, the batch, or a portion thereof, is in a vessel. In some embodiments, the batch, or portion thereof, is in a vial. In some embodiments, the batch, or portion thereof, is in a plurality of vials. In some embodiments, the batch, or portion thereof, is in a syringe.

In some embodiments, the batch, or a portion thereof, is in a plurality of vials, wherein each vial comprises a protein, wherein the fucosylated and afucosylated forms of the protein are at a predetermined ratio. In some embodiments, the batch, or a portion thereof, is in a plurality of vials, wherein at least about 50%, 55%, 60%, 65%, 70%, 75%, 80%, 85%, 90%, 95%, or 100% of the vials comprise fucosylated and afucosylated forms of a protein are at a predetermined ratio.

In some embodiments, the batch is aliquoted into a unit dosage. As used herein, a “unit dosage” is the amount of protein intended for administration as a single unit dose. In some embodiments, the single unit dose is about 1 to about 500 mg of a protein. In some embodiments, the unit dosage is packaged in a container. In some embodiments, the unit dosage is packaged in a vial.

### Commercial Manufacturing

In some embodiments, the host cells of the present disclosure may be employed in the production of a molecule of interest at manufacturing scale. “Manufacturing scale” production of therapeutic proteins, or other proteins, utilize cell cultures ranging from about 400 L to about 80,000 L, depending on the protein being produced and the need. Typically, such manufacturing scale production utilizes cell culture sizes from about 400 L to about 25,000 L. Within this range, specific cell culture sizes such as 4,000 L, about 6,000 L, about 8,000, about 10,000, about 12,000 L, about 14,000 L, or about 16,000 L, about 25,000 L may be utilized.

In some embodiments, the host cells of the present disclosure, e.g., host cells with a protein-expression polynucleotide inserted into the host cell genome at a targeted integration (TI) site can be employed in the production of large quantities of a molecule of interest in a shorter timeframe as compared to non-TI cells. In certain embodiments, the host cells of the present disclosure can be employed for improved quality of the molecule of interest as compared to non-TI cells used in current cell culture methods. In certain embodiments, the host cells of the present disclosure can be used to enhance seed train stability by preventing chronic toxicity that can be caused by products that can cause cell stress and clonal instability over time. In certain embodiments, the host cells of the present disclosure can be used for the optimal expression of acutely toxic products.

In certain embodiments, the host cells, the TI systems of the present disclosure, can be used for cell culture process optimization and/or process development.

In certain embodiments, the host cells of the present embodiment can be employed to reduce aggregate levels of a molecule of interest as compared to non-TI cells used in conventional cell culture methods.

In certain embodiments, the host cells of the present disclosure can be used to achieve increased expression of a polypeptide (or polypeptides) of interest relative to a host cell where the exogenous sequence expressing the polypeptide (or polypeptides) of interest is randomly integrated. For example, but not by way of limitation, the host cells of the present disclosure can achieve expression of standard and half antibodies at titers of at least 3 g/L, 3.5 g/L, 4 g/L, 4.5 g/L, 5 g/L, 5.5 g/L, 6 g/L, 6.5 g/L, 7 g/L, 7.5 g/L, 8 g/L, 8.5 g/L, 9 g/L, 9.5 g/L, 10 g/L, 10.5 g/L, 11 g/L, or more, and expression of multispecific antibodies, e.g., bispecific antibodies, of at least 1.5 g/L, 2 g/L, 2.5 g/L, 3 g/L, 3.5 g/L, 4 g/L, 4.5 g/L, 5 g/L, 5.5 g/L, 6 g/L, or more. In certain embodiments, the host cells of the present disclosure can achieve increased bispecific content relative to a host cell where the exogenous sequence(s) expressing the bispecific content is randomly integrated. For example, but not by way of limitation the host cells of the present disclosure can achieve bispecific content of at least 80%, 85%, 90%, 95%, 96%, 98%, 99% or more.

In certain embodiments, the host cells of the present disclosure can be used as an investigational tool. In certain embodiments, the host cells of the present disclosure can be used as a diagnostic tool to map out the root causes of low protein expression for problematic molecules in various cells. In certain embodiments, the host cells of the present disclosure can be used to directly link an observed phenomenon or cellular behavior to the transgene expression in the cells. The host cell of the present disclosure can also be used to demonstrate whether or not an observed behavior is reversible in the cells. In certain embodiments, the host cells of the present disclosure can be exploited to identify and mitigate problems with respect to transgene(s) transcription and expression in cells.

In some embodiments, the size of the commercial batch is no greater than about 1, 2, 3, 4, 5, 6, 7, 8, 9, or 10 times the size of the clinical batch. As used herein, “commercial batch” refers to an amount of protein produced during one or more production runs completed for purposes of commercial production and/or distribution. As used herein, “clinical batch” refers to an amount of protein produced during one or more production runs completed for purposes of clinical testing. In some embodiments, the size of the commercial batch is no greater than 10 times the size of the clinical batch.

In some embodiments, the vessel comprises an aliquot of a commercial batch as described herein, wherein the commercial batch comprises a composition comprising a protein, wherein the fucosylated and afucosylated forms of the protein are present at a predetermined ratio. In some embodiments, the vial comprises an aliquot of a commercial batch as described herein, wherein the commercial batch comprises a composition comprising a protein, wherein the fucosylated and afucosylated forms of the protein are present at a predetermined ratio. In some embodiments, an aliquot of a commercial batch as described herein is comprised within a syringe, wherein the commercial batch comprises a composition comprising a protein, wherein the fucosylated and afucosylated forms of the protein are at a predetermined ratio.

### Pharmaceutical Compositions

The protein produced by the methods of the invention may be formulated into a pharmaceutical composition.

According to particular embodiments, the method of the first or second or sixth aspects further comprise the step of formulating the protein into a pharmaceutical composition comprising a pharmaceutically acceptable excipient or diluent.

In some embodiments, the pharmaceutical composition is in a form suitable for storage. In some embodiments, the pharmaceutical composition is in a form suitable for product transportation. In some embodiments, the pharmaceutical composition is frozen. In some embodiments, the pharmaceutical composition is lyophilized. In some embodiments, the pharmaceutical composition is reconstituted. In some embodiments, the pharmaceutical composition is an administration composition. In some embodiments, the pharmaceutical composition is in a form for administration to an individual in need thereof.

In some embodiments, the pharmaceutical composition is a sterile pharmaceutical composition. Sterile pharmaceutical formulations are compounded or manufactured according to pharmaceutical-grade sterilization standards (e.g., United States Pharmacopeia Chapters 797, 1072, and 1211; California Business & Professions Code 4127.7; 16 California Code of Regulations 1751, 21 Code of Federal Regulations 21, or ex-U.S. counterparts to such regulations) known to those of skill in the art.

In some embodiments, the pharmaceutical composition is a stable formulation. As used herein, “stable” formulation is one in which the proteins therein essentially retain physical and chemical stability and integrity upon storage. Various analytical techniques for measuring protein stability are available in the art and are reviewed in, for example, Jones, Adv Drug Delivery Rev, 10, 1993. Stability can be assessed at a selected temperature for a selected time period. For example, the extent of aggregation during storage can be used as an indicator of protein stability. Thus, a “stable” formulation may be one wherein less than about 10% and preferably less than about 5% of the protein are present as an aggregate in the formulation.

In some embodiments, the pharmaceutical composition is a reconstituted formulation. As used herein, a “reconstituted” formulation is one which has been prepared by dissolving a lyophilized protein formulation in a diluent such that the protein is dispersed throughout. The reconstituted formulation is suitable for administration (e.g. intravenous or sub-cutaneous administration) to an individual in need there.

In some embodiments, the pharmaceutical composition is an isotonic formulation. As used herein, an “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood.

In some embodiments, the pharmaceutical composition is at a specified pH. In some embodiments, the pharmaceutical composition is at a pH of about 5-7, about 5-6, or about 5-5.5. In some embodiments, the pharmaceutical composition is at a pH of about 5.3. In some embodiments, the pharmaceutical composition is at a pH of about 5.4. In some embodiments, the pharmaceutical composition is pH adjusted.

In some embodiments, the pharmaceutical composition further comprises a pharmaceutically acceptable carrier. As used herein, a “pharmaceutically acceptable carrier” refers to an ingredient in a pharmaceutical composition, other than an active ingredient. A pharmaceutically acceptable carrier includes, but is not limited to, a buffer, excipient, stabilizer, or preservative. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In some embodiments, the pharmaceutically acceptable carrier is selected from the group consisting of sodium acetate, sucrose, polysorbate (e.g., polysorbate 20), sodium succinate, histidine HCl, and sodium chloride.

In some embodiments, the pharmaceutical composition further comprises a pharmaceutically acceptable acid. As used herein, a “pharmaceutically acceptable acid” includes inorganic and organic acids which are non-toxic at the concentration and manner in which they are formulated. For example, suitable inorganic acids include hydrochloric, perchloric, hydrobromic, hydroiodic, nitric, sulfuric, sulfonic, sulfinic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxy acetic, trifluoroacetic, phenylacetic, trimethylacetic, t-butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2,2,2]-oct-2-ene-1-carboxylic, glucoheptonic, 4,4′-methylenebis-3-(hydroxy-2-ene-1-carboxylic acid), hydroxynapthoic.

In some embodiments, the pharmaceutical composition further comprises a pharmaceutically acceptable base. As used herein, a “pharmaceutically acceptable base” includes inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic nontoxic bases including, primary, secondary and tertiary amines, substituted amines, cyclic amines and basic ion exchange resins, for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases usable with the present invention include those which are derived from the amino acids, for example, histidine, glycine, phenylalanine, aspartic acid, glutamic acid, lysine and asparagine.

In some embodiments, the pharmaceutical composition further comprises a pharmaceutically acceptable buffer or salt, for example, those derived from both acid and base addition salts of the above indicated acids and bases. Specific buffers and/or salts include histidine, succinate and acetate.

In some embodiments, the pharmaceutical composition further comprises a pharmaceutically acceptable sugar. As used herein, a “pharmaceutically acceptable sugar” is a molecule which, when combined with a protein, significantly prevents or reduces chemical and/or physical instability of the protein upon storage. When the formulation is intended to be lyophilized and then reconstituted, “pharmaceutically acceptable sugars” may also be known as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

In some embodiments, the pharmaceutical composition further comprises a pharmaceutically acceptable preservative. As used herein, a “pharmaceutically acceptable preservative” is a compound which can be added to the formulations herein to reduce bacterial activity.

Examples of potential preservatives include, but are not limited to, octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Materials and Methods

### Vector Constructs and Reagents

Constitutive antibody expression was achieved by cloning the heavy and light chain genes each under the control of a separate cytomegalovirus (CMV) promoter in a targeted integration (TI) CHO platform (Ng, D., et al., Biotechnol Prog, 2021. 37(4): p. e3140). Two vectors (front and back) each containing one copy of heavy chain (HC) and two copies of light chain (LC) molecules, with puromycin as selection marker, were integrated into the TI hotspot, resulting in a total of 2 HC and 4 LC molecules integrated into each cell after puromycin (Ng, D., et al., Biotechnol Prog, 2021. 37(4): p. e3140). Lens culinaris agglutinin (LCA) (Cat #L-1040) and fluorescein isothiocyanate (FITC) conjugated LCA (LCA-FITC) (Cat #FL-1041) reagents were purchased from Vector Laboratories. Fucose was purchased from Vector Laboratories (Cat #S-9007). The following primers were used for amplifying the WT or FXKO gene.

Forward WT FX primer:
(SEQ ID NO: 1)
GTCACCCAAAGCTCTCCTTG,
reverse WT FX primer:
(SEQ ID NO: 2)
GAAGTCCATGGCCTCCACTA,
forward FXKO primer:
(SEQ ID NO: 3)
TTCCAGGGGATGTACTCTGC,
reverse FXKO primer:
(SEQ ID NO: 4)
TAAATGCTCACCTCCGCTCT.

### Knocking Out the FX Gene by CRISPR/Cas9 Technology

Six guide RNA (gRNA) sequences were designed flanking the 5′ and 3′ regions of the FX gene and were used following three rounds of transfection to completely or partially delete a 6 kb section at the 5′ region of the FX gene as described (Cynthia Lam, Biotechnol Prog. 2024 Apr. 17:e3471. doi: 10.1002/btpr.3471), followed by single cell cloning. Clones containing complete or partial KO of the FX gene were identified by FACS analysis using LCA-FITC staining as described (Salina Louie, Biotechnol Bioeng. 2017 March; 114(3):632-644. doi: 10.1002/bit.26188). Clones with complete KO of the FX gene were further confirmed by PCR analysis as described (Salina Louie, Biotechnol Bioeng. 2017 March; 114(3):632-644. doi: 10.1002/bit.26188).

| Description | sgRNA sequence | SEQ ID NO |
|---|---|---|
| sgRNA-1F | TACCTAGTAGGCTCATGACT | SEQ ID NO: 5 |
| sgRNA-1R | TCCCTACGACTGAAAAATCA | SEQ ID NO: 6 |
| sgRNA-6F | TTCCGTTTGTGGTCACACAC | SEQ ID NO: 7 |
| sgRNA-6R | AGATCCAGCTCAGACTGTTG | SEQ ID NO: 8 |
| sgRNA-6F2 | GCCTCTCTGCCTCAGACTTG | SEQ ID NO: 9 |
| sgRNA-6R2 | GTGTGTGACCACAAACGGAA | SEQ ID NO: 10 |

### Cell Culture and Production Assay

CHO cells were cultured in chemically defined media and were grown in 37° C. and 5% CO2. Cells were passaged at seeding densities of 4×105 cells/ml every 3-4 days. Fed-batch production cultures were performed in AMBR®15 (TAP Biosystems) using Genentech serum free chemically defined media. Cells were seeded at 2×106 cells/mL on Day 0 with set points of temperature 36° C., DO 40%, pH 7.2, and an agitation rate of 1400 rpm. Cultures were temperature shifted from 36° C. to 35° C. on Day 3 and from 35° C. to 33° C. on Day 6. Cultures received feed medium on Days 3, 6, and 9. Cells were harvested after 12 days (Hsu W T, Aulakh R P, Traul D L, Yuk I H. Cytotechnology. 2012; 64(6):667-678). To determine the range of afucosylation mAb-1 that could be produced by the FX KO clones, fucose was added to a final concentration of 1 mM to the media. For these cultures, fucose addition started on either Days 0, 1, 3, 4, 5, 6, 7, or 8. Once a culture received fucose, additional fucose was fed on normal feeding days: Days 3, 6, and 9, when applicable.

### Off-Line Sample Analyses

Cultures were assayed throughout the 12-day production process for viable cell count (VCC), viability, pH, and lactate using a BioProfile FLEX2 instrument (Nova Biomedical). Glucose was measured daily using a BioProfile FLEX2 instrument. Antibody titer and product quality assays were measured using cell culture supernatant samples purified by PhyTip protein A column. Antibody glycan distribution was analyzed by capillary electrophoresis with fluorescence detection. All protein product quality assays were developed in-house (Hsu W T, Aulakh R P, Traul D L, Yuk I H. Cytotechnology. 2012; 64(6):667-678).

### Example 1. CHO Clones with Partial KO of the FX Gene can Express a Fixed Level of Afucosylated mAb without Fucose Addition

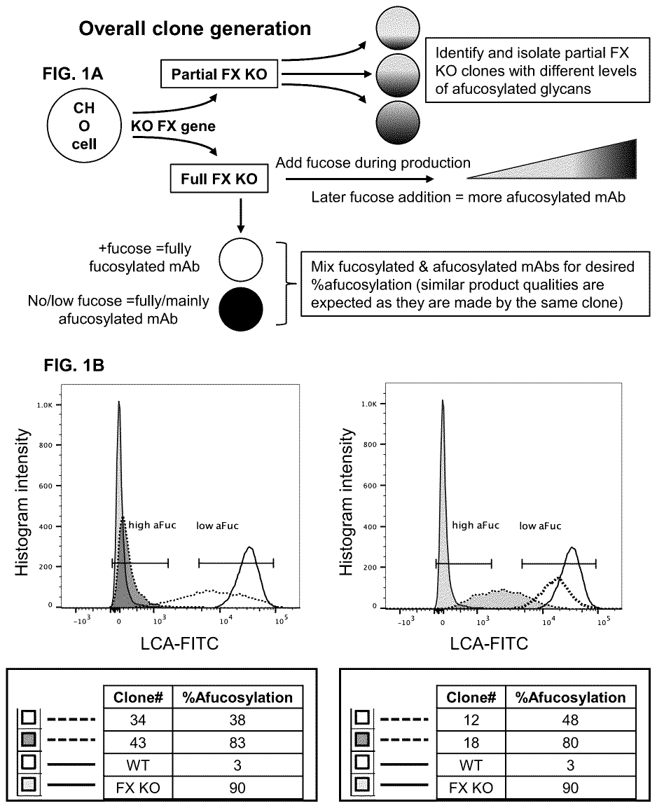

To evaluate the advantages of partial vs. complete KO of the FX gene, CRISPR-Cas9 technology was used to target deletion of FX gene in a mAb-1 expressing cell line (FIG. 1A).

Clones with varying degrees of partial and complete KO of the FX gene were identified.

Clones with partial KO of the FX gene were found to have a phenotypic range of fucosylation depending on the level of FX protein expression and its activity, while clones with full KO of the FX gene make either fucosylated or afucosylated mAb-1 depending on the presence or absence of fucose in the media (FIG. 1A).

LCA-FITC binds to fucosylated surface proteins and can be used to measure fucosylation levels on cells using FACS analysis. We noticed that for the clones with the partial KO of the FX gene the LCA-FITC staining profiles seem to be approximately proportional to the levels of afucosylated mAb-1 expressed. In general, the LCA-FITC FACS profiles that fell within a specified range proximal to the FX KO host FACS profile seemed to have more than 50% afucosylated mAb-1 species, while the ones that were within a specific range proximal to the WT host FACS profile had less than 50% afucosylated mAb-1 (FIG. 1B). For example, as depicted in FIG. 1B, clones 43 and 18 had LCA-FITC profiles that trended more towards the FX KO host profile and they expressed about 80% afucosylated mAb-1 in an AMBR®15 cell culture production run. On the other hand, clones 34 and 12 had LCA-FITC profiles that looked more similar to the WT host profile and made about 40-50% afucosylated mAb-1 antibody during production (FIG. 1B).

To further confirm this observation, several mAb-1 expressing clones with partial KO of the FX gene were identified based on LCA-FITC staining profiles and were evaluated in AMBR®15 cell culture production. AMBR®15 is an automated small scale (15 ml volume) micro-bioreactor system that allows running 24-48 parallel samples at the 10-15 mL microbioreactor scale in one run.

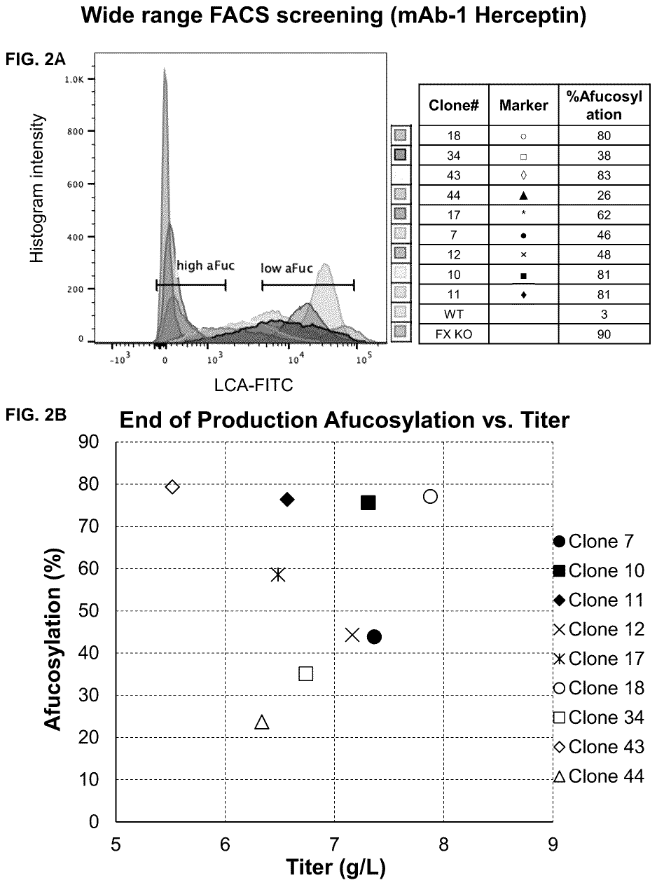

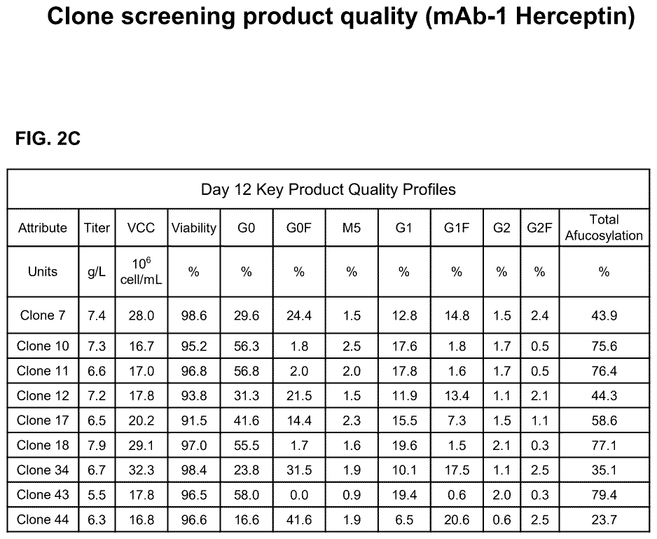

On average, clones with LCA-FITC profiles closer to the marked high aFuc range expressed 60-80% afucosylated mAb-1, while clones with FACS profiles that were closer to low aFuc mark expressed 26-48% afucosylated mAb-1 (see FIG. 2A for the markings).

Complete FX KO and WT hosts expressed >approx. 90% and approx. 3% total afucosylated mAb-1, respectively (FIG. 2A). We did not observe a clear correlation between % afucosylated mAb-1 species and clone titers. Irrespective of the mAb-1 afucosylation levels, partial FX KO clones had antibody expression titers of 6.3-7.9 g/L (FIG. 2B and FIG. 2C). All partial FX KO clones had comparable growth and viabilities during AMBR®15 production and were capable of expressing a fixed percentage of afucosylated mAb-1 without addition of fucose to the production cultures (FIG. 2C).

These findings indicated that partial KO of the FX gene could allow isolation of clones with potentially a desired level of afucosylated mAb without addition of fucose or other culture manipulations during production.

If a clone with a partial KO of FX gene capable of expressing a protein of interest, e.g. mAb, with a desired or near desired ratio of fucosylated to afucosylated forms is selected/utilised then adjustment of the amount of fucose in the culture medium can be used to fine tune the system to produce the desired ratio of fucosylated to afucosylated forms, For example, if the selected partial KO cell capable of expressing the protein of interest, results in a 50% ratio of fucosylated:afucosylated protein, but the desired ratio is 65% fucosylated:afucosylated protein the addition of a small amount of fucose to the culture medium (or a slightly greater amount than normally used) can shift to the desired 65% fucosylated:afucosylated protein (i.e., addition of fucose will increase the amount of fucosylated form produced).

### Example 2. Addition of Fucose to the FX KO CHO Clones at Different Timepoints During Production can Robustly and Tightly Control Levels of Afucosylated mAb

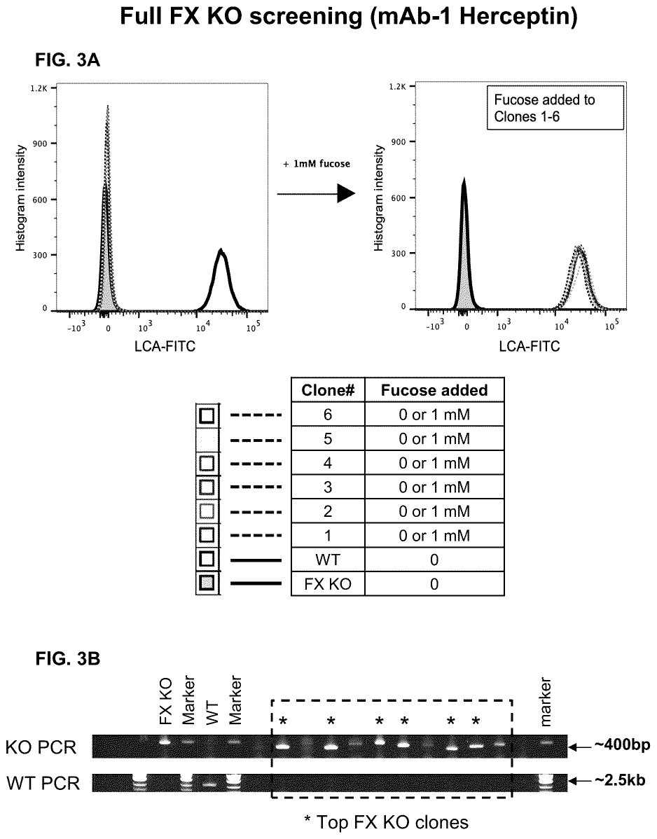

LCA-FTIC staining and FACS analysis of six mAb-1 expressing CHO clones cultured with or without fucose (1 mM) was used to identify clones with complete KO of the FX gene. In the absence of fucose, LCA-FITC FACS profiles of these clones (depicted in dashed lines) perfectly matched the FACS profile of a previously identified (Salina Louie, Biotechnol Bioeng. 2017 March; 114(3):632-644. doi: 10.1002/bit.26188) FX KO host (bold and solid line, filled gray), indicating that FX gene is fully knocked out in these clones (FIG. 3A, left panel). By culturing the very same FX KO clones in a media containing 1 mM fucose for 2 days, their LCA-FITC FACS profiles (dashed lines) completely shifted, becoming indistinguishable from that of the WT host (bold and solid line, no fill), in accordance to what was previously reported (Salina Louie, Biotechnol Bioeng. 2017 March; 114(3):632-644. doi: 10.1002/bit.26188) (FIG. 3A, right panel). Knockout of the FX gene in these clones was also confirmed by PCR analysis where only a PCR product for the FX KO but not WT alleles were detected (FIG. 3B). This further confirmed that the FX gene has been completely deleted in all the alleles in these FX KO clones.

To evaluate whether fucose addition at different time points during production can be utilized to adjust afucosylated mAb-1 levels, three full FX KO clones (clones 1, 4, and 5) were used to set up AMBR®15 production cultures. Fucose (1 mM final) was then added at different days during production followed by fucose addition during applicable feeds.

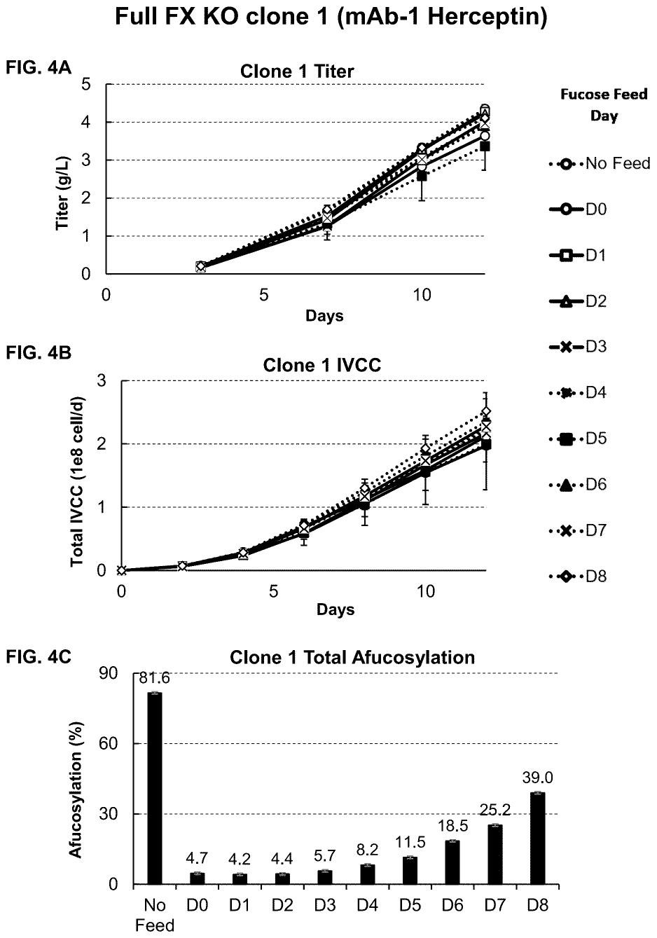

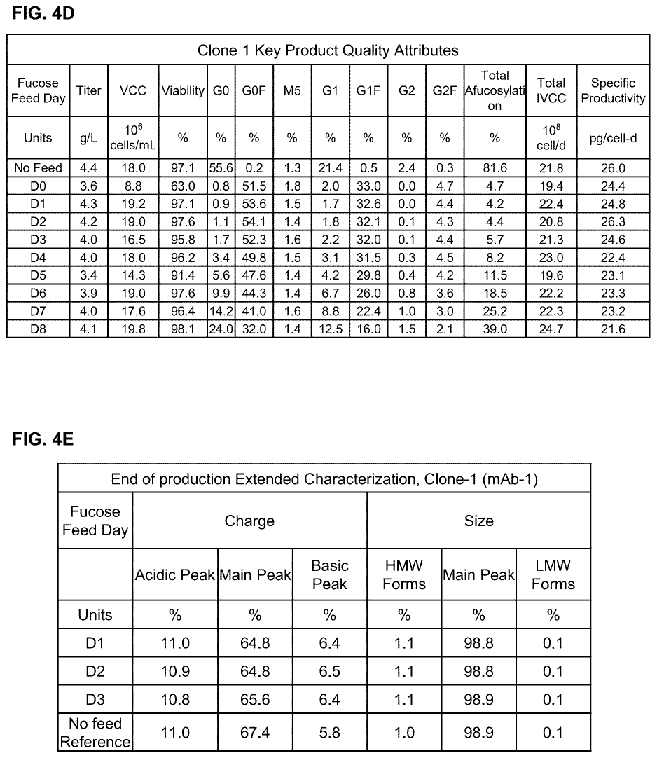

For clone 1, the culture titers (FIG. 4A) and growth (FIG. 4B) were mostly within a certain range (3.5-4.4 g/L) for all conditions whether fucose was added to the cultures or not (FIG. 4A). However, a very incremental and controllable increase in the levels of afucosylated mAb-1 was observed with fucose feed in these cultures. The early addition of fucose to the cultures (between days 0 to 2) resulted in lowest levels of % afucosylation (4.2-4.7%) while addition of fucose in subsequent days gradually increased the % afucosylated mAb-1 levels from 5.7% for day 3 to 39% for day 8 fucose addition (FIG. 4C). Percentages of major glycan species as well as culture performance for all fucose feed conditions is depicted in FIG. 4D.

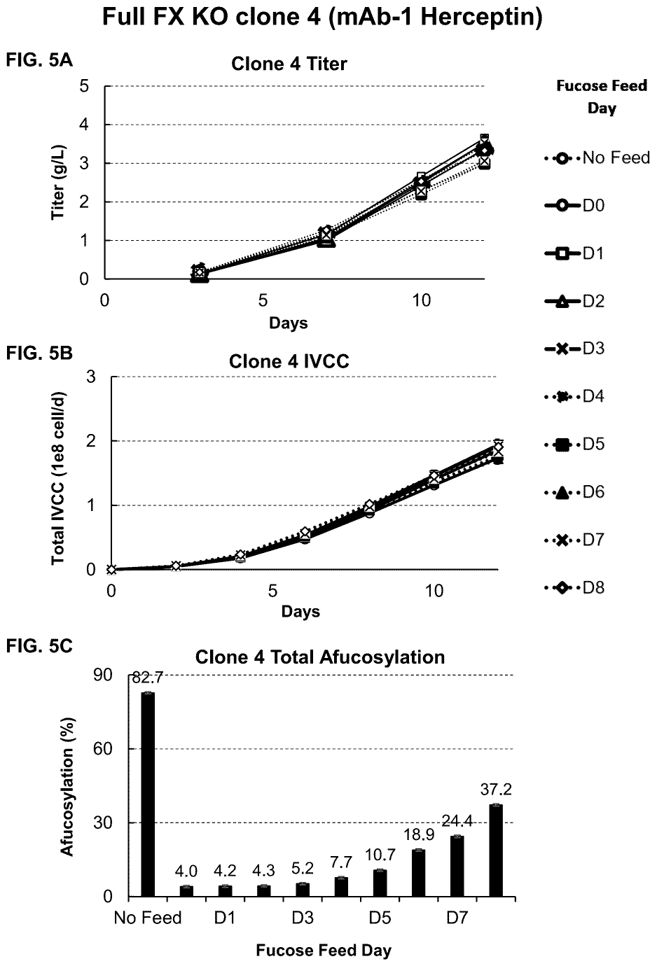

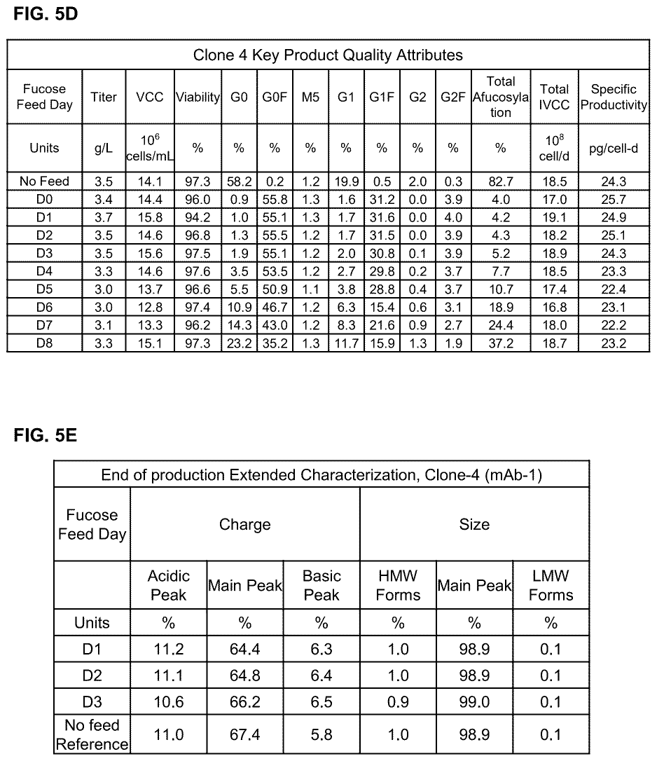

Similar results were observed for clone 4, where the titers (ranging from 3-3.7 g/L) and growth for all different conditions were on average comparable (FIG. 5A and FIG. 5B) and gradual increase from 4.2% to 37% afucosylated mAb-1 were observed as fucose addition to the culture was delayed from day 2 to day 8, respectively (FIG. 5C and FIG. 5D). Addition of fucose at different days (days 3 to 8) resulted in incremental increases in % afucosylated mAb-1, allowing identification of culture conditions in which tightly controllable levels of afucosylated mAb can be achieved.

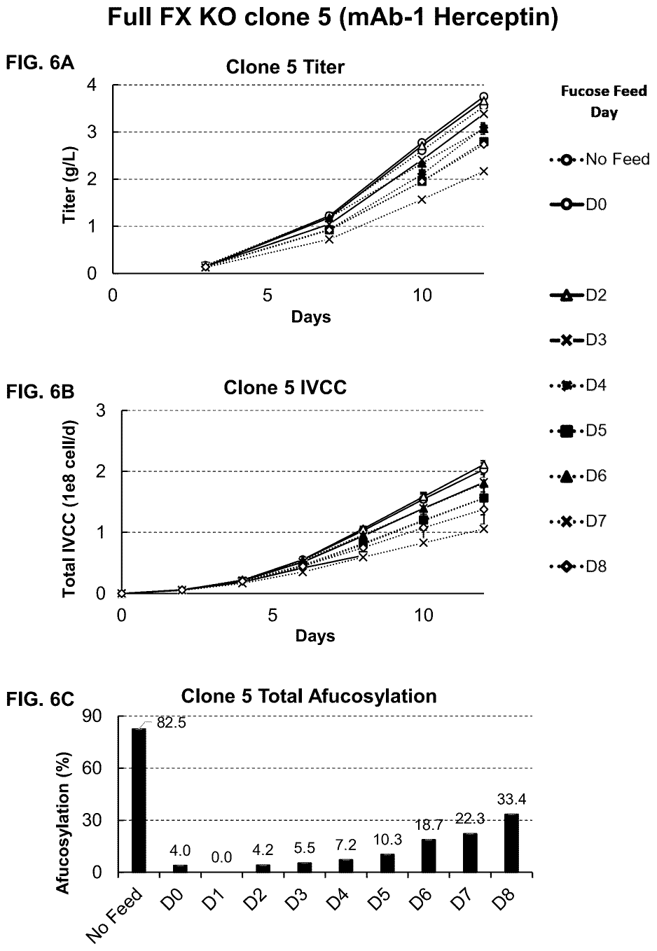

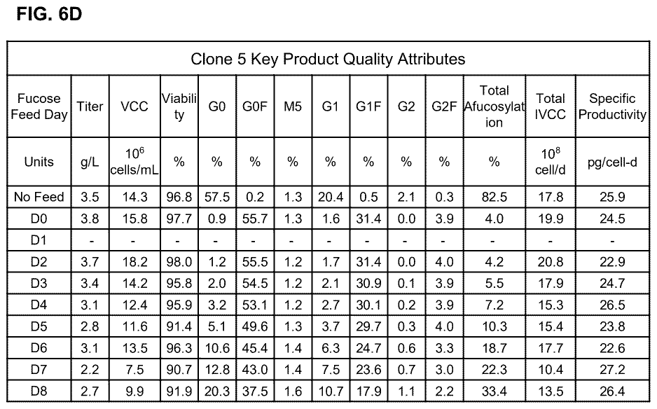

For clone 5 somewhat of a wider range with regards to titer and growth was observed in cultures where fucose was added later during production. In general about 10-20% lower titers were observed in cultures with later fucose feed compared to the cultures that received fucose addition earlier (FIG. 6A and FIG. 6D). The observed lower titers for clone 5 in cultures that received a fucose feed later during production was mainly due to lower rates of growth (FIG. 6B and FIG. 6D). The harvest titers for clone 5 ranged from 2.2-3.8 g/L (FIG. 6A) but irrespective of the wider titer ranges, these cultures had % afucosylated mAb-1 levels ranging from 4.2-33.4% (FIG. 6C and FIG. 6D), which was within similar ranges observed for clones 1 and 4 (FIG. 4A, FIG. 4B, FIG. 4C, FIG. 4D, and FIG. 4E; and FIG. 5A, FIG. 5B, FIG. 5C, FIGS. 5D, and 5E). Therefore, in all 3 tested clones, approximately similar levels of % afucosylated mAb-1 species were observed and this was mainly dependent on the day of fucose addition during the production run. This is due to the fact that % accumulation of afucosylated mAb species is directly depended on specific productivity rather than titer and specific productivity of all tested clones remained unchanged in all conditions, irrespective of fucose addition day (FIG. 4D, FIG. 5D, and FIG. 6D).

### Example 3 Clones Bearing Partial Knockout of the FX Gene have a Very Tight Modulation of Afucosylated Antibody Expression when Fucose is Added to the Production Culture

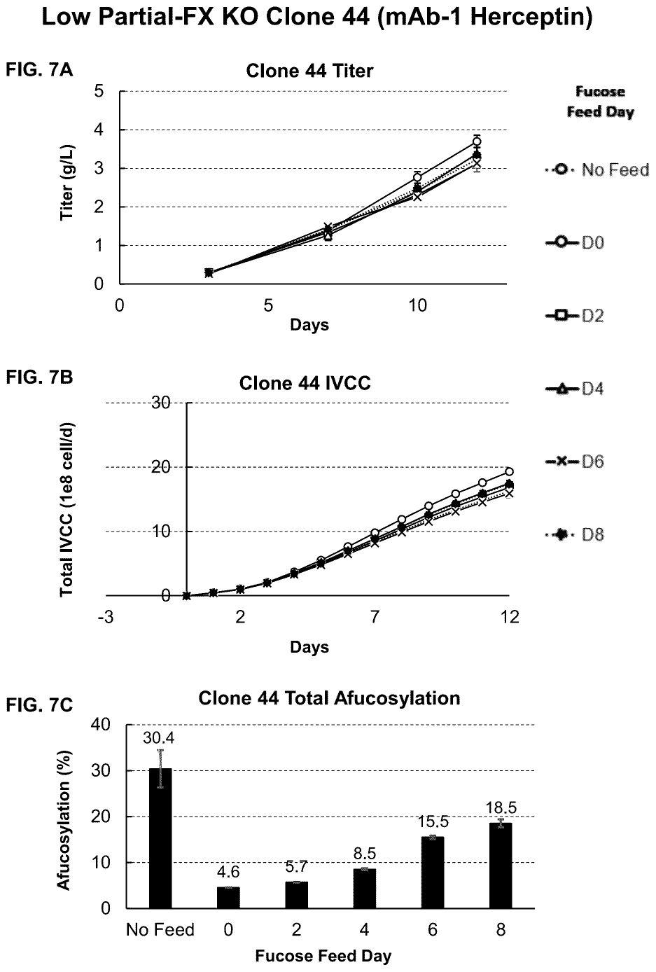

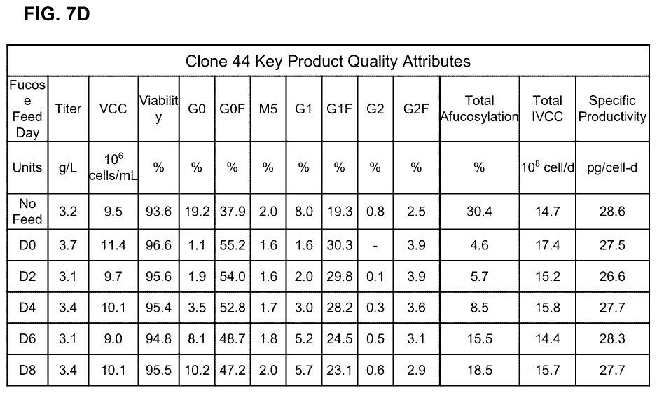

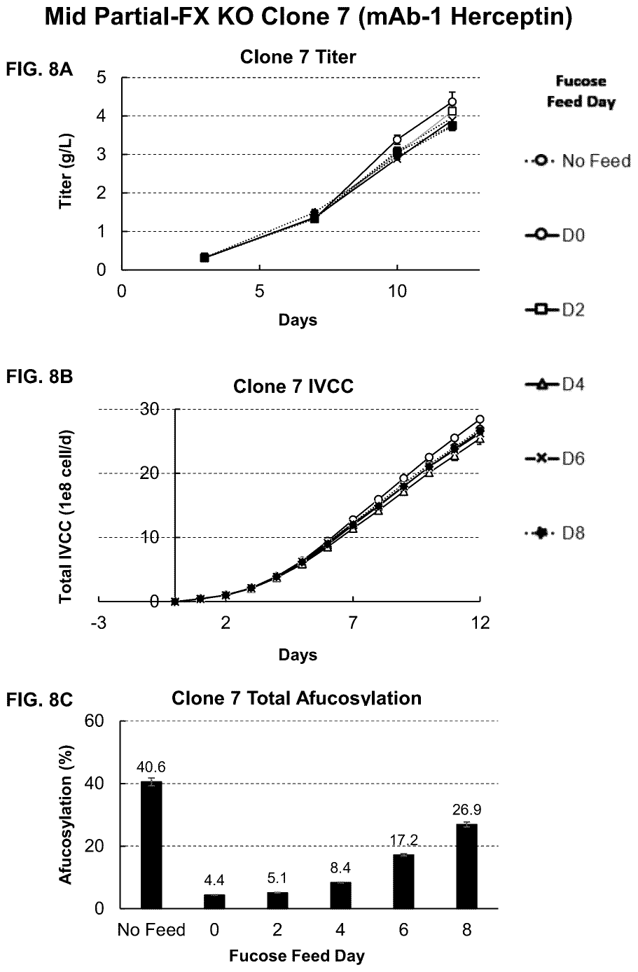

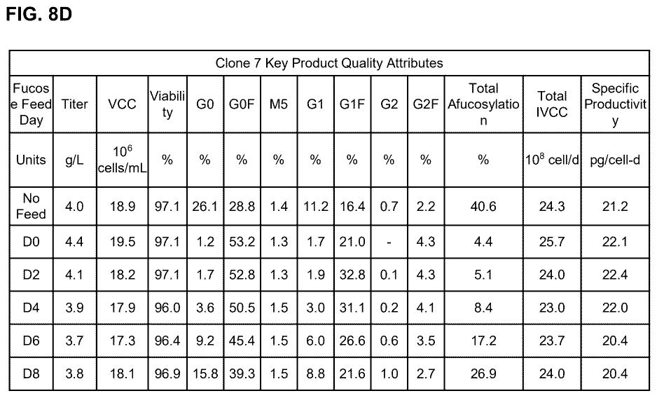

Our earlier findings (FIG. 2A, FIG. 2B, and FIG. 2C) revealed that clones with partial KO of the FX gene express much lower levels of afucosylated antibodies compared to the clones bearing complete knockout of the FX gene. Hence, it was reasoned that addition of fucose at different time points during production to a clone bearing partial KO of the FX gene might allow for a tighter and more controlled modulation of % afucosylated antibody expression. To test this, two partial FX KO clones, one expressing lower (26%) levels of afucosylated mAb-1 (clone 44, FIG. 2A, FIG. 2B, and FIG. 2C) and one expressing medium (46%) levels of afucosylated mAb-1 (clone 7, FIG. 2A, FIG. 2B, and FIG. 2C) were evaluated in a production assay in which fucose (1 mM final) was added at different days during production as explained above. Addition of fucose during production process did not have any negative impact on culture titers in partial FX KO clones with low (clone 44, FIG. 7A) or medium (clone 7, FIG. 8A) levels of % afucosylated mAb-1. Clone 44 cultures with fucose addition on different days achieved a titer range of 3.1-3.7 g/L (FIG. 7D) while clone 7 cultures had a titer range of 3.7-4.4 g/L (FIG. 8D). Similarly, addition of fucose did not adversely affect culture growth in either clone 44 (FIG. 7B) or clone 7 (FIG. 8B). Addition of fucose (1 mM) at different days (days 0 to 8) to low partial FX KO clone 44 incrementally increased % afucosylated mAb-1 levels from 4.6-18.5% (FIG. 7C) while for mid partial FX KO clone 7 the % afucosylated mAb-1 levels ranged from 4.4-26.9% (FIG. 8C). Details of major glycan species as well as culture performance for all fucose feed conditions for low and mid partial FX KO clones 44 and 7 are captured in FIG. 7D and FIG. 8D, respectively.

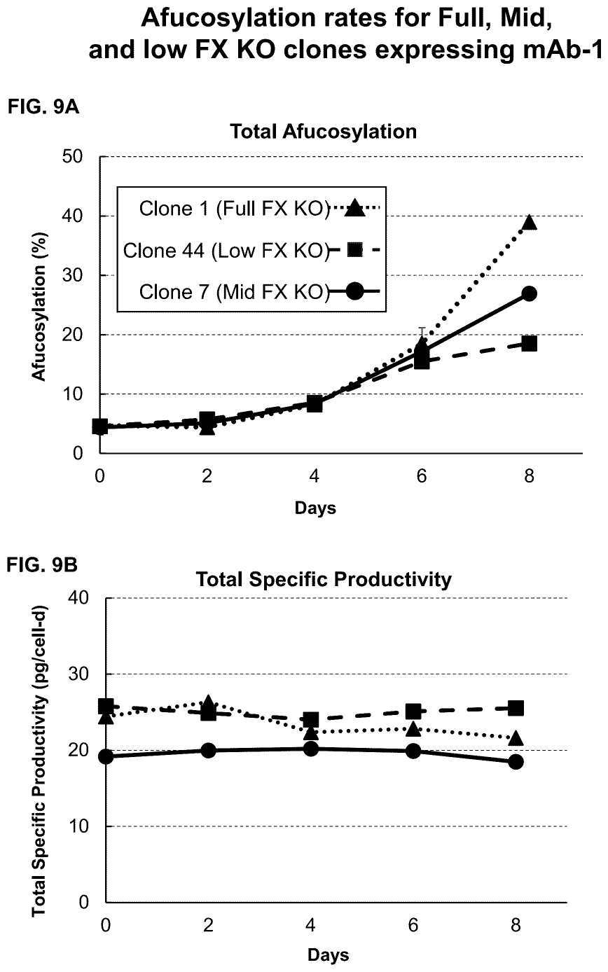

For comparison purposes and to clearly depict the impact of low, mid, and full FX KO clones on expression of afucosylated mAb-1, the levels of afucosylated mAb-1 expressed by clones 1 (Full FX KO clone), 44 (low partial FX KO), and 7 (mid partial FX KO) during production were graphed together (FIG. 9A). Addition of fucose (1 mM) during production on days 2 and 4 resulted in comparable overall mAb-1 afucosylated glycan profiles for all the tested clones (FIG. 9A) as the levels of antibody expression is very minor during the first 3-4 days of standard fed-batch production. Starting from day 6 fucose addition conditions, these clones began to differentiate from one another with regards to afucosylated mAb-1 expression and by day 8 clear differences in % afucosylated mAb-1 levels were obvious. Full FX KO clone 1 had as high as 39% afucosylated mAb-1 when fucose was added on day 8 of the fed-batch production culture, while partial mid (clone 7) and low (clone 44) FX KO clones showed only 26.9 and 18.5% afucosylated mAb-1 levels. Depicting total specific productivity of clones with full (clone 1), mid partial (clone 7) and low partial (clone 44) levels of FX gene knockout revealed no significant fluctuations in their specific productivities due to fucose addition at different days during production (FIG. 9B).

### Example 4 Regulation of Afucosylated mAb Levels by FX KO CHO Hosts is not Molecule Specific

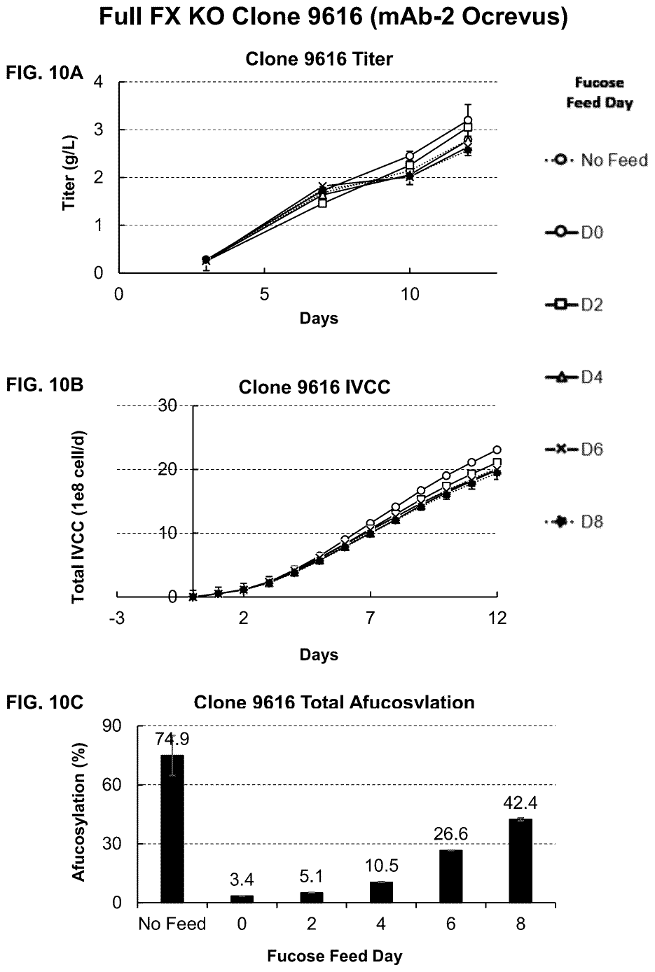

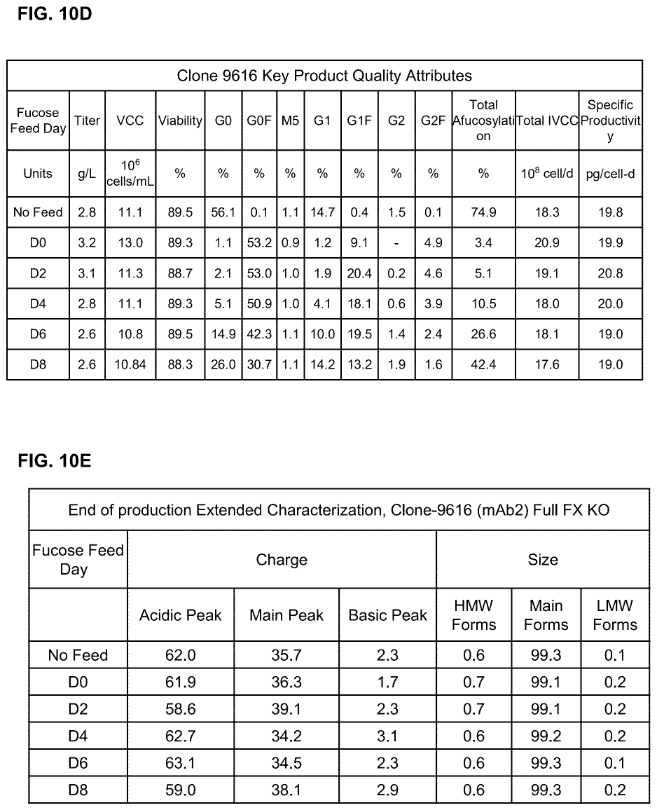

To ensure that the observed regulation of antibody fucosylation levels is not molecule specific, a second CLD using FX KO CHO host was performed to express mAb-2 in this host. This mAb-2 expressing FX KO CHO culture (clone 9616) was evaluated in a production assay with no fucose feed or with fucose feed (1 mM final) added on days 0, 2, 4, 6, or 8. Similar to what was previously observed, fucose addition to the production cultures did not impact productivity and all cultures had a titer range of 2.6-3.2 g/L (FIG. 10A). Culture growth was also comparable among all the tested conditions, irrespective of their differences in fucose addition/feed strategy (FIG. 10B). Likewise, addition of fucose at different days during the production process resulted in controlled titration of % afucosylated mAb-2 levels ranging from 3.4-42.4% in full FX KO clone 9616 (FIG. 10C). Levels of major glycan species and other culture performance parameters for all the tested fucose feed conditions remained comparable (FIG. 10D). Furthermore, product quality attributes for all the tested conditions were obtained and independent of fucose addition day or feed, no major differences in levels of acidic/main/basic charge variant species or HMW/LMW forms were observed in these cultures (FIG. 10E).

### Example 5 Mixing Fully Afucosylated and Highly Fucosylated Culture Media can be Used to Achieve the Desired Levels of % Afucosylated mAb Levels

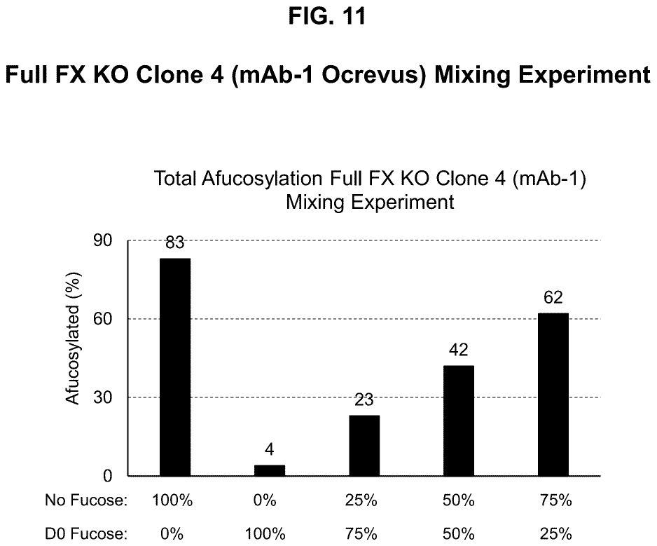

A unique advantage of utilizing full FX KO CHO hosts for vcell line development lies in the fact these hosts are capable of expressing completely afucosylated (no fucose feed) or mainly fucosylated (Day 0 fucose feed) mAbs with comparable product quality attributes. Hence, by performing two separate production runs, one with no fucose feed and one with Day 0 fucose feed, to express both fully afucosylated and mainly fucosylated versions of mAbs. We reasoned that by mixing certain ratios of harvested cell culture fluids (HCCF) from these samples a desired level of % afucosylated mAb species can be achieved. To this end, HCCF samples from fully afucosylated and mainly fucosylated production cultures (Full FX KO clone 4 study, FIG. 5), were mixed at 1:3, 1:1, and 3:1 ratio and their glycan profiles, with respect to total % afucosylated species, were analyzed (FIG. 11). Interestingly, the theoretical and experimental target values of % afucosylated mAb species very closely matched each other (FIG. 11). Therefore, mixing HCCFs, from cultures with fully afucosylated or mainly fucosylated mAb species, can indeed be a viable option to make drug products with exact desired levels of % afucsoylated mAb species.

### Discussion

Afucosylated mAb species play an important role in MOA of therapeutic antibodies as they can increase NK cells mediated killing of the target cells by 10-100 folds. Here we have shown that LCA-FITC FACS profiles can be used to isolate CHO clones with partial KO of the FX gene capable of expressing a fixed level of % afucosylated mAbs without addition of fucose or any other culture manipulations (FIG. 1B, FIG. 2A, FIG. 2B, and FIG. 2C). Additionally, we have developed a process in which CHO clones with full (FIG. 4, FIG. 5, FIG. 6, and FIG. 10) or partial (FIG. 7 and FIG. 8) KO of the FX gene can be utilized, in a fed-batch production culture, to express precise and desired levels of afucosylated mAbs by adding fucose to the culture. In this production process fucose was added to the culture and feed on particular days during production and at saturating levels (1 mM) to completely switch the CHO cells from expressing afucosylated mAbs to fucosylated mAbs (FIG. 3, FIG. 4, FIG. 5, FIG. 6, FIG. 7, FIG. 8, FIG. 9, and FIG. 10). The product quality attributes such as % charge variant or high molecular species levels for all these samples were comparable, irrespective of fucose addition day during production (FIG. 4E, FIG. 5E, and FIG. 10E). Having comparable product quality attributes is very critical since they might impact the data interpretation with regards to any observed changes in a particular antibody's or set of antibodies' mode of action, as it cannot be directly linked to % afucosylated mAb levels. This method of generating afucosylated mAbs harbors many benefits over the traditional approaches. For example, using the FUT8 KO strategy requires generation of two separate cell lines, one in the WT and the other in the FUT8 KO background hosts, in order to express both fucosylated and afucosylated antibodies. In this approach, however, identification of clones with comparable product quality attributes would pose a major challenge, especially since these cell lines are derived from different hosts (WT vs. FUT8 KO). Chemical inhibitors have also been utilized to control protein afucosylation levels. Use of such inhibitors might negatively impact culture growth, covalently modify the mAb molecules (data not shown), and cannot achieve fully afucosylated mAb levels at moderate concentrations^{10 11}.

It was also observed that while full FX KO clones generated the highest % afucosylated mAbs (FIG. 4C, FIG. 5C, and FIG. 6C) compared to clones bearing partial KO of the FX gene (FIG. 7C and FIG. 8C), the rate of % afucosylated mAb expression in partial FX KO clones were significantly slower than clones with full FX KO genotype (FIG. 9A). This would make partial FX KO clones a more attractive option for expression of mAbs with a desired level of % afucosylated species. It should be noted that the maximum levels of % afucosylated mAb expression by a partial FX KO clone is capped at the % afucosylation levels that it can achieve when fucose is not added to the culture and feed media during the production process. Hence, depending on the levels of % afucosylated mAb species desired, one needs to select candidate clones that are screened for achieving the anticipated target % afucosylated mAb levels.

Interestingly, it was also observed that different full FX KO clones could generate approximately similar levels of % afucosylated mAb-1, irrespective of their titer differences (FIG. 4C, FIG. 5C, FIG. 6A, FIG. 6B, FIG. 6C, and FIG. 6D). This observation suggested that irrespective of growth or titer differences amongst different FX KO clones, subjection of these clones to the proposed production process would allow seamless and robust expression of a certain level of % afucosylated mAb depending only on the day of fucose feed. This is likely due to the fact that expression of a certain % afucosylated mAb species is dependent mainly on a clone's specific productivity. Therefore, so long as the overall productivity of the clones do not change during the duration of the production process, the % afucosylated mAb-1 levels should remain comparable among different clones. Indeed, our findings confirmed that all the tested clones showed minor specific productivity fluctuations throughout the production process (FIG. 9B, FIG. 4D, FIG. 5D, FIG. 6D, FIG. 7D, and FIG. 8D). This stability in specific productivity is critical for predicting/estimating the levels of afucosylated mAb that can be achieved during production and is owed to the fact that fucose addition does not negatively impact culture performance during production, may not be true when using small molecule inhibitors to block fucosylation pathways in CHO cells. Additionally, stability and lot to lot variability of small molecule inhibitors can also negatively impact generation of afucosylated mAbs, making it harder to predict exactly what levels of % afucosylated mAbs can be expressed from a given culture.

As shown above and previously reported, full FX KO CHO hosts can be strategically used to express fully afucosylated or mainly fucosylated (>95%) mAbs by simply not adding or adding fucose at day 0 to the production culture, respectively. Here we confirmed that indeed different ratios of harvested cell culture fluids from fully afucosylated or substantially fucosylated cultures can be mixed prior to protein-A column purification to precisely achieve the desired levels of % afucosylated mAbs (FIG. 11). This feature of the FX KO hosts can be taken advantage of in a manufacturing setting where after performing two separate production bioreactors (with or without fucose feed), a single process can be used for the rest of the downstream processes such as purification, analytical, formulation, and filling. In short, altogether, our findings confirmed that utilizing CHO clones with partial or full KO of the FX gene and in the proposed production process can seamlessly be utilized for expression of mAbs with a wide range and desired % afucosylated glycan species.

What is claimed is:

1. A method of producing a protein with a desired ratio of fucosylated to afucosylated glycan forms, the method comprising:
(a) culturing a host cell capable of expressing a protein of interest in a culture medium under conditions suitable to express the protein of interest, wherein the host cell has been engineered to (i) produce the protein of interest, and (ii) to possess reduced or inactivated GDP-keto-6-deoxymannose-3,5-epimerase, 4-reductase (FX) activity compared to wild type,
wherein the amount of FX activity in the host cell influences the ratio of fucosylated to afucosylated glycan forms of the expressed protein.

2. The method according to claim 1, wherein the method further comprises:
b) determining the level of fucosylation of the expressed protein; and
c) adjusting the amount of the fucose source in the culture medium so that the protein is produced with the desired level of fucosylation.

3. A method of producing a protein, wherein the protein is produced in fucosylated and afucosylated forms in a desired ratio, comprising:
culturing a host cell capable of expressing a protein of interest in fucosylated and afucosylated forms in a desired or near-desired ratio in a culture medium, wherein the host cell has a partial knock-out of the FX gene, optionally wherein the near-desired ratio of fucosylated to afucosylated forms is adjusted to the desired ratio by increasing or decreasing the amount of fucose in the culture medium and adjusting the timing of the fucose addition.

4. The method according to claim 3, wherein the ratio of fucosylated to afucosylated forms of the expressed protein of interest is influenced by the amount of FX activity produced by the cell with partial FX KO.

5. The method according to any one of claims 1-4, wherein the FX activity of the host cell is between 25% and 75% of wild-type activity.

6. The method of according to any one of claims 1-5, wherein the host cell capable of expressing the protein of interest in fucosylated and afucosylated forms in a desired or near-desired ratio has been selected from a plurality of cells subjected to partial knock out of FX gene using gene editing engineering.

7. The method according to any one of the preceding claims, wherein the cell engineered to possess reduced GDP-keto-6-deoxymannose-3,5-epimerase,4-reductase (FX) activity compared to wild type is capable of expressing the protein of interest.

8. The method according to any one of claims 1-6, wherein the cell engineered to partially inactivate the FX gene is further modified to express the protein of interest.

9. The method according to claim 8, wherein the cell subjected to gene editing to partially inactivate the FX gene is further modified to express the protein of interest by transforming or transfecting the gene edited cell with one or more nucleic acid molecules capable of expressing the protein of interest.

10. The method of any one of the preceding claims, wherein the host cell capable of expressing a protein of interest has been produced by the method of claim 22.

11. The method according to any one of the preceding claims, wherein the culture medium comprises a fucose source.

12. The method according to claim 11, wherein the fucose source is a fucose, in particular wherein (i) the fucose is L-fucose, (ii) the fucose is L-fucose-1-phosphate, or (iii) the fucose source is GDP-fucose.

13. The method according to any one of the preceding claims, wherein the cell is cultured in a fed-batch process, an intensified fed-batch process, a continuous or a semicontinuous process.

14. The method according to any one of claims 11 to 13, wherein the fucose source is added to the culture medium via bolus addition, via continuous feed or fed-batch feed.

15. The method according to any one of the preceding claims, wherein the protein is an Fc-containing protein.

16. The method according to claim 15, wherein the Fc-containing protein comprises an antibody heavy chain or a fragment thereof.

17. The method according to claim 15 or 16, wherein the Fc-containing protein is a full length antibody, in particular wherein the full length antibody is a monoclonal antibody.

18. The method according to any one of the preceding claims, wherein the host cell is a eukaryotic cell, and/or wherein the host cell is a mammalian cell, in particular wherein the mammalian cell is a Chinese hamster ovary (CHO) cell.

19. The method according to claim 18, wherein the host cell is a CHO cell selected from the group consisting of a DP12 cell, a DUXB-11 derived DHFR-deficient DP12 cell, a CHO-K1 cell, a DHFR-positive CHO-K1 cell, and a CHOK1M cell.

20. The method according to any one of the preceding claims, further comprising purifying the protein from the culture medium.

21. The method according to claim 20, further comprising formulating the protein into a pharmaceutical composition comprising a pharmaceutically acceptable excipient or diluent.

22. The method according to any one of the preceding claims, wherein the host cell has been engineered to possess reduced FX activity compared to wild type by introducing a sequence deletion, a sequence addition or a sequence substitution into the FX gene in the host cell causing a partial knock out of the FX gene.

23. The method according to claim, wherein the sequence deletion, sequence addition or sequence substitution to the FX gene has been created using: (a) a clustered, regularly interspaced, short palindromic repeats (CRISPR) system; (b) a transcription activator-like effector nuclease (TALEN) system; (c) a zinc-finger nuclease (ZFN) system; (d) a transposase system; or (e) a meganuclease system.

24. A method of producing a cell capable of expressing a protein of interest, wherein the protein of interest can be produced in fucosylated and afucosylated forms in a desired ratio, comprising:
(i) subjecting isolated host cells capable of expressing a protein of interest to gene editing to produce individual gene-edited cell clones with complete to partial knock-out of the FX gene;
(ii) selecting the gene-edited cell which produces the protein of interest in fucosylated and afucosylated forms at the desired ratio; or
optionally,
(i) transfecting a clonal population of an isolated host cell that has been gene edited to have reduced FX activity compared to wild type with a polynucleotide sequence encoding a protein of interest, wherein the isolated host cell has been selected for a particular level of FX activity which results in an expressed protein being produced in a particular ratio of fucosylated and afucosylated forms;
(ii) selecting a transfected cell clone which expresses the protein of interest in fucosylated and afucosylated forms at a desired ratio.

25. The method according to claim 24, wherein the gene editing is selected from: (a) a clustered, regularly interspaced, short palindromic repeats (CRISPR) system; (b) a transcription activator-like effector nuclease (TALEN) system; (c) a zinc-finger nuclease (ZFN) system; (d) a transposase system; or (e) a meganuclease system.

26. The method according to any one of the preceding claims, wherein the level of fucosylation or the ratio of fucosylated and afucosylated protein produced by a cell clone is determined using LCA-FITC staining and FACS profile analysis.

27. A cell clone capable of expressing a protein of interest produced by the method of claim 24 or 25.

28. A cell culture comprising the cell clone according to claim 27 and a culture medium, the culture medium optionally comprising a source of fucose.

29. The cell culture according to claim 28, further comprising afucosylated and fucosylated forms of a protein of interest.

30. A method of producing a protein, wherein the protein is produced in fucosylated and afucosylated forms at a desired ratio, comprising
(i) determining the period of time required for culturing a host cell having substantially no FX activity in a culture medium lacking a fucose source prior to culturing said host cell in a culture medium comprising a fucose source sufficient to produce fucosylated and afucosylated forms of the protein in a desired ratio;
(ii) culturing the host cell having substantially no FX activity in a culture medium lacking a fucose source for the period of time determined in step (i); and
(iii) culturing the host cell in a culture medium comprising a fucose source for a period of time sufficient to produce the protein in fucosylated and afucosylated forms at the desired ratio.

31. The method according to claim 30, further comprising purifying the protein from the culture medium.

32. The method according to claim 31, further comprising formulating the protein into a pharmaceutical composition comprising a pharmaceutically acceptable excipient or diluent.

33. The method according to claim 30, wherein the cell is cultured in a fed-batch process, an intensified fed-batch process, a continuous or a semicontinuous process.

34. The method according to claim 30, wherein the culture medium comprising the fucose source is produced by adding a fucose source to the culture medium lacking a fucose source.

35. The method according to claim 34, wherein the fucose source is added via bolus addition.

36. The method according to claim 34, wherein the fucose source is added via continuous feed or fed-batch feed.

37. The method according to claim 36, wherein the fucose source is added via continuous feed or fed-batch feed so as to maintain the concentration of fucose in the culture medium at about 1 mM.

38. The method according to any one of claims 34-37, wherein the amount of the added fucose source in the culture medium is between 0.01 mM and 10 mM.

39. The method of any one of claims 30-38, wherein the fucose source is a fucose, in particular wherein (i) the fucose is L-fucose, (ii) the fucose is L-fucose-1-phosphate, or (iii) the fucose source is GDP-fucose.

40. The method of any one of claims 30-39, wherein the protein is an Fc-containing protein or a fusion protein.

41. The method of claim 40, wherein the Fc-containing protein comprises an antibody heavy chain or a fragment thereof or a fusion protein.

42. The method of claim 40 or 41, wherein the Fc-containing protein is a full length antibody, in particular wherein the full length antibody is a monoclonal antibody, a bispecific- or a trispecific-antibody, or a complex antibody.

43. The method of any one of claims 30-42, wherein the host cell is a eukaryotic cell, and/or wherein the host cell is a mammalian cell, in particular wherein the mammalian cell is a Chinese hamster ovary (CHO) cell.

44. The method of claim 43, wherein the host cell is a CHO cell selected from the group consisting of a DP12 cell, a DUXB-11 derived DHFR-deficient DP12 cell, a CHO-K1 cell, a DHFR-positive CHO-K1 cell, and a CHOK1M cell.

45. The method of any one of claims 30-44, wherein the host cell has substantially no FX activity due to the FX gene in the host cell being knocked out by a sequence deletion or by a sequence addition or substitution.

46. The method of claim 45, wherein the FX gene has been knocked out using: (a) a clustered, regularly interspaced, short palindromic repeats (CRISPR) system; (b) a transcription activator-like effector nuclease (TALEN) system; (c) a zinc-finger nuclease (ZFN) system; (d) a transposase system; or (e) a meganuclease system.

47. The method of any one of claims 30-46, wherein the host cell having substantially no FX activity is cultured in the culture medium lacking a fucose source for up to 14 days.

48. The method of any one of claims 30-47, wherein initiation of the culturing of the host cell having substantially no FX activity in the culture medium comprising a fucose source occurs between 0 and 14 days after initiation of the culturing of the host cell in the culture medium lacking a fucose source.

49. The method of any one of claims 1-48, wherein the protein of interest is expressed in the host cell.

50. The method of claim 49, wherein the protein of interest is introduced into the host cell genome either randomly or in a targeted manner.


SEQUENCE LISTING (10 sequences)

SEQ ID NO: 1 - DNA, 20 bp, synthetic construct
  source 1..20: mol_type = other DNA
gtcacccaaagctctccttg

SEQ ID NO: 2 - DNA, 20 bp, synthetic construct
  source 1..20: mol_type = other DNA
gaagtccatggcctccacta

SEQ ID NO: 3 - DNA, 20 bp, synthetic construct
  source 1..20: mol_type = other DNA
ttccaggggatgtactctgc

SEQ ID NO: 4 - DNA, 20 bp, synthetic construct
  source 1..20: mol_type = other DNA
taaatgctcacctccgctct

SEQ ID NO: 5 - RNA, 20 nt, synthetic construct
  source 1..20: mol_type = other RNA
tacctagtaggctcatgact

SEQ ID NO: 6 - RNA, 20 nt, synthetic construct
  source 1..20: mol_type = other RNA
tccctacgactgaaaaatca

SEQ ID NO: 7 - RNA, 20 nt, synthetic construct
  source 1..20: mol_type = other RNA
ttccgtttgtggtcacacac

SEQ ID NO: 8 - RNA, 20 nt, synthetic construct
  source 1..20: mol_type = other RNA
agatccagctcagactgttg

SEQ ID NO: 9 - RNA, 20 nt, synthetic construct
  source 1..20: mol_type = other RNA
gcctctctgcctcagacttg

SEQ ID NO: 10 - RNA, 20 nt, synthetic construct
  source 1..20: mol_type = other RNA
gtgtgtgaccacaaacggaa